(19) United States
(12) Patent Application Publication
(10) Pub. No.: US 2009/0305963 A1
(43) Pub. Date: Dec. 10, 2009

(54) Lipocalin 2 for the Treatment, Prevention, and Management of Cancer Metastasis, Angiogenesis, and Fibrosis

(76) Inventors: Vikas P. Sukhatme, Newton, MA (US); S. Ananth Karumanchi, Chestnut Hill, MA (US); Pankaj Seth, Newton, MA (US); Junichi Hanai, Boston, MA (US); Tadanori Mammoto, Brookline, MA (US); Jonathan Barasch, New York, NY (US); Kiyoshi Mori, Sakyo-ku (JP)

Correspondence Address: CLARK & ELBING LLP, 101 FEDERAL STREET, BOSTON, MA, 02110, US

(21) Appl. No.: 11/795,392

(22) PCT Filed: Jan. 19, 2006
(86) PCT No.: PCT/US2006/001738

§ 371 Date: Feb. 12, 2009

Related U.S. Application Data
(60) Provisional application No. 60/645,438, filed on Jan. 19, 2005.

(51) Int. Cl. A61K 38/17; A61P 35/00; C12Q 1/68; G01N 33/574; A61P 7/04

(52) U.S. Cl. 514/12; 435/6; 436/86

(57) ABSTRACT

The invention features methods and compositions for treating and preventing cancer metastasis, angiogenic disorders, and fibrotic disorders using lipocalin 2 compounds.

### FIELD OF THE INVENTION

In general, this invention relates to lipocalin 2 compounds and methods of using lipocalin 2 compounds for the treatment and diagnosis of various diseases, including cancer metastasis, angiogenic disorders, and fibrotic disorders.

### BACKGROUND OF THE INVENTION

Lipocalin 2, also known as neutrophil gelatinase-associated lipocalin (NGAL) is a member of a superfamily of carrier proteins that is expressed in granulocytic precursors as well as in numerous epithelia cell types. Crystallography shows that the protein is a carrier of iron bound to a siderophore, which is a small organic molecule produced by bacteria (Goetz et al., Mol Cell 10:1033-1043, 2002). Lipocalin 2 has been implicated in a diverse array of physiological processes including apoptosis and iron transport.

Several disease processes have been demonstrated to involved the transition of cells from an epithelial cell type to a mesenchymal cell type, a process known as epithelial to mesenchymal transition (EMT), or a transition from a mesenchymal cell type to an epithelial cell type, a process known as mesenchymal to epithelial transition (MET). EMT is involved in a variety of disease-related processes including cancer metastasis, angiogenesis, and fibrosis. For example, in cancer, metastatic disease occurs when the disseminated foci of tumor cells seed a tissue which supports their growth and propagation, and this secondary spread of tumor cells is responsible for the morbidity and mortality associated with the majority of cancers. EMT allows the cells to convert from a polarized cell to a non-polar, mobile cell, a transition critical to the metastatic process. There is a pressing need for therapies that target events, such as EMT, that lead to cancer metastasis. At present only chemotherapy and in a few cancers, immune based therapies address this need. While the few existing therapies available aim to treat cancer metastasis, they do not prevent the occurrence of metastasis.

Fibrosis and angiogenesis are also examples of cellular processes that can be associated with various disorders. Angiogenesis is the formation of new blood vessels and is associated with a number of cancer-related and cancer-unrelated disorders. For example, inappropriate angiogenesis can be involved in the pathogenesis of cancer metastasis, rheumatoid arthritis, chronic inflammation, and ocular neovascular diseases and there is also a need for anti-angiogenic agents that can be used for the treatment of any disorder involving inappropriate angiogenesis. There is also a continuing need for new anti-fibrotic agents. Fibrosis is the abnormal accumulation of fibrous tissue that can occur as a part of the wound-healing process in damaged tissue. Such tissue damage may result from physical injury, inflammation, infection, exposure to toxins, and other causes. While the formation of fibrous tissue is part of the normal beneficial process of healing after injury, in some circumstances there is an abnormal accumulation of fibrous materials such that it may ultimately lead to organ failure. Many of the diseases associated with the proliferation of fibrous tissue are both chronic and often debilitating, including for example, skin diseases such as scleroderma, dermal scar formation, keloids, liver fibrosis, bone marrow fibrosis, cardiac fibrosis, lung fibrosis (e.g., silicosis, asbestosis), kidney fibrosis (including diabetic nephropathy), and glomerulosclerosis. Some, including pulmonary fibrosis, can be fatal due in part to the fact that the currently available treatments for this disease have significant side effects and are generally not efficacious in slowing or halting the progression of fibrosis. There are currently no effective therapies for the prevention of fibrosis, which ultimately leads to organ failure and death in cases of kidney failure, cirrhosis, among others.

### SUMMARY OF THE INVENTION

We have discovered that lipocalin 2, an iron-siderophore binding protein reverses the EMT, and can be used to treat or prevent any disorder associated with EMT, including cancer metastasis, fibrosis, and angiogenesis. We have also discovered that lipocalin 2 suppresses cell invasiveness, blocks VEGF production, and induces thrombospondin, thereby inhibiting many of the signaling pathways and processes that contribute to angiogenesis and metastasis. Lipocalin 2, and biologically active fragments and derivatives thereof, can therefore be used to treat, prevent, or reduce metastatic disease, angiogenesis, or fibrosis.

Accordingly, in a first aspect, the invention features a method of treating or preventing metastasis in a subject having cancer, that includes administering to the subject a lipocalin 2 compound, or a fragment or derivate thereof, that has lipocalin 2 biological activity, for a time and in an amount sufficient to treat or prevent the metastasis. In one embodiment, the lipocalin 2 compound is a lipocalin 2 polypeptide, or fragment or derivative thereof, and can include a sequence that is substantially identical (e.g., at least 60%, 70%, 80%, 90%, 95%, 96%, 97%, 98%, 99%, or 100% identical) to the amino acid sequence of SEQ ID NOs: 2 or 4. Desirably, the sequence includes or consists of a sequences that is identical to the sequence of SEQ ID NOs: 2 or 4. Lipocalin 2 compounds can also include a nucleic acid molecule encoding a lipocalin 2 polypeptide that has lipocalin 2 biological activity. Desirably, the lipocalin 2 nucleic acid molecule encodes a polypeptide having substantially identity to the amino acid sequence of SEQ ID NOs: 2 or 4. The nucleic acid molecule can include a sequence substantially identical to the nucleic acid sequence of SEQ ID NOs: 1 or 3. Preferably the nucleic acid molecule includes or consists of a sequence that is identical to the sequence of SEQ ID NOs: 1 or 3.

Additional useful lipocalin 2 compounds include any peptidyl or non-peptidyl compound that is a lipocalin 2 analog and has or induces lipocalin 2 biological activity; any peptidyl or non-peptidyl compound that binds to a lipocalin 2 receptor (e.g., 24p3R in mouse cells; see Devireddy et al., Cell 123:1293-1305, 2005); any peptidyl or non-peptidyl lipocalin 2 receptor agonists, including but not limited to agonistic antibodies; any compound known to stimulate or increase blood serum levels of lipocalin 2 polypeptides or increase the biological activity of lipocalin 2 polypeptides; any compound known to decrease the expression or biological activity of a lipocalin 2 inhibitor (e.g., an inhibitor that blocks binding to a siderophore or a lipocalin 2 receptor); and any compound that mimics lipocalin 2 effects on reducing raf, MEK, or ERK1/2 phosphorylation and/or biological activity.

Preferred lipocalin 2 polypeptides, fragments or derivatives thereof, or non-peptidyl lipocalin 2 compounds will have at least 25%, 30%, 40%, 50%, 60%, 70%, 80%, 90%, 95%, 96%, 97%, 98%, 99%, or more lipocalin 2 biological activity. Non-limiting examples of lipocalin 2 biological activity include siderophore or iron-siderophore binding; reversal of EMT, as described herein; lipocalin 2 receptor binding (Devireddy et al., Cell 123:1293-1305, (2005)); inhibition of ras-MAPK signaling pathway; reduction of E-cadherin phosphorylation; induction of E-cadherin expression or biological activity; induction of E-cadhelin degradation; reduction of VEGF expression, induction of thrombospondin 1 expression, retinol transport; cryptic coloration; olfaction; pheromone transport; prostaglandin synthesis; and apoptosis (see Akerstrom et al., Biochim. Biophy. Acta 1482:1-8, 2000; and Flower et al., Biochem. J. 318:1-14, 1996). Assays for lipocalin 2 biological activity include assays for siderophore binding, iron transport, iron uptake (e.g., analysis of expression of ferritin protein levels and colorimetric determination of intracellular iron), and receptor binding, as described in Hanai et al., (J. Biol. Chem. 280:13641-13647, (2005)), Mori et al., (J. Clin. Invest. 115:610-621 (2005)), Li et al., (Am. J. Cell Physiol. 287:C1547-1559 (2004)), Yang et al., Mol. Cell (10:1045-1056 (2002)), and Devireddy et al., supra, and apoptotic assays known in the art. Preferably, the lipocalin 2 compound has siderophore or iron-siderophore binding activity and can transport iron and can reverse EMT by at least 25%, 30%, 40%, 50%, 60%, 70%, 80%, 90%, 95%, 96%, 97%, 98%, 99%, or more.

In preferred embodiments, the method further includes administering a siderophore to the subject. Non-limiting examples of siderophores are bacterial catecholate-type ferric siderophores, enterochelin, carboxymycobactin, aminochelin, desferrioxamine, aerobactin, arthrobactin, schizokinen, foroxymithine, pseudobactins, neoenactin, photobactin, ferrichrome, hemin, achromobactin, achromobactin, and rhizobactin. The siderophore can be administered alone, or in a pre-formed complex with lipocalin 2 and/or iron. The method can also include administering iron or an iron replacement therapy to the subject with or without the siderophore. For example, the method can include administering lipocalin 2 and iron; lipocalin and iron in a pre-formed complex; lipocalin, a siderophore and iron; or lipocalin, a siderophore, and iron in a pre-formed complex. Preferred iron replacements include ferrous sulfate and ferrous fumarate or dextran-iron for IV use and these can be administered orally or intravenously, as needed.

The cancer can be a solid tumor or a non-solid or soft tissue tumor. In preferred embodiments of the above aspect, the tumor is an epithelial cell solid tumors (e.g., tumors of the gastrointestinal tract, colon, breast, prostate, lung, kidney, liver, pancreas, ovary, head and neck, oral cavity, stomach, duodenum, small intestine, large intestine, anus, gall bladder, labium, nasopharynx, skin, uterus, male genital organ, urinary organs, bladder, and skin).

The method can be used, for example, to treat metastasis or reduce the size or extent of the metastasis in a metastatic cancer, to prevent or reduce the likelihood of metastasis in a subject having a primary cancer that is at risk of becoming metastatic, or as a preventive measure in a subject having an increased risk for metastatic cancer (e.g, a subject having a known BRCA1 or 2 mutation). The method may be used in conjunction with additional anti-cancer therapies including, surgery, radiation therapy, chemotherapy, differentiating therapy, immune therapy, anti-angiogenic and anti-proliferative therapy. For these combination therapies, the lipocalin 2 compound can be administered before during, or after, or any combination thereof, the additional anti-cancer therapy. Examples of each of these anti-cancer therapies are described below.

In a second aspect, the invention features a kit for the treatment or prevention of metastasis in a subject having or at risk of developing metastatic cancer. The kit includes a lipocalin 2 compound and instructions for the use of the lipocalin 2 compound for the treatment of prevention of metastatic cancer. The kit can also include an additional anti-cancer compound such as a chemotherapeutic agent, an angiogenesis inhibitor, or an anti-proliferative agent.

In a third aspect, the invention features a method for reducing or inhibiting angiogenesis in a subject in need thereof. The method includes administering to the subject a lipocalin 2 compound for a time and in an amount sufficient to reduce or inhibit the angiogenesis.

The method can be used to reduce or inhibit angiogenesis in a subject having cancer, preferably a metastatic cancer or a cancer at risk for becoming metastatic. The method can also be used to reduce or inhibit angiogenesis in a subject having an angiogenic disorder such as inflammatory disorders such as immune and non-immune inflammation, rheumatoid arthritis, ocular neovascular disease, choroidal retinal neovascularization, osteoarthritis, chronic articular rheumatism, psoriasis, disorders associated with inappropriate or inopportune invasion of vessels such as diabetic retinopathy, neovascular glaucoma, restenosis, capillary proliferation in atherosclerotic plaques and osteoporosis, cancer associated disorders, such as solid tumors, solid tumor metastases, hematopoetic tumors or metastases, angiofibromas, retrolental fibroplasia, hemangiomas, Kaposi's sarcoma, and cancers or cancer metastases, which require neovascularization to support tumor growth. The method can also include administering at least one additional angiogenic inhibitor. Examples of angiogenic inhibitors are described herein.

In a fourth aspect, the invention features a kit for the treatment or prevention of angiogenesis in a subject having, or at risk of developing, an angiogenic disorder. The kit includes a lipocalin 2 compound and instructions for the use of the lipocalin 2 compound for the treatment or prevention of angiogenesis. The kit can also include at least one additional compound, such as a chemotherapeutic agent, an angiogenesis inhibitor, or an anti-proliferative compound.

In yet another aspect, the invention features a method for treating or preventing fibrosis in a subject having a fibrotic disorder, that includes administering to the subject a lipocalin 2 compound for a time and in an amount sufficient to prevent or reduce the occurrence of fibrosis. Non-limiting examples of fibrotic disorders are described herein.

Desirably, the lipocalin 2 compound is applied to the surface or under the surface of medical devices. The method can also include admininistering at least one additional anti-fibrotic agent (e.g., agent that blocks TGF-β signaling or inhibits activation of plasminogen activator inhibitor-1 promoter activity, an antibody that binds to TGF-β, or to a TGF-β receptor, an antibody that binds to TGF-β receptor I, II, or III, a kinase inhibitor, an agent that blocks connective tissue growth factor (CTGF) signaling, an agent that inhibits prolyl hydroxylase, an agent that inhibits procollagen C-proteinase, pirfenidone, silymarin, pentoxifylline, colchicine, embrel, remicade, an agent that antagonizes TGF-β, an agent that antagonizes CTGF, and an agent that inhibits vascular endothelial growth factor VEGF).

In another aspect, the invention features a kit for the treatment or prevention of fibrosis in a subject having, or at risk of developing, a fibrotic disorder, that includes a lipocalin 2 compound and instructions for the use of the lipocalin 2 compound for the treatment or prevention of the fibrotic disorder. The kit can also include one or more additional anti-fibrotic agents.

In preferred embodiments of any of the therapeutic aspects (methods and kits) of the invention, the lipocalin 2 compound is a lipocalin 2 polypeptide, or fragment or derivative thereof, and can include a sequence that is substantially identical (e.g., at least 60%, 70%, 80%, 90%, 95%, 96%, 97%, 98%, 99%, or 100% identical) to the amino acid sequence of SEQ ID NOs: 2 or 4. Desirably, the sequence includes or consists of a sequence identical to the sequence of SEQ ID NOs: 2 or 4. Lipocalin 2 compounds can also include a nucleic acid molecule encoding a lipocalin 2 polypeptide that has lipocalin 2 biological activity. Desirably, the lipocalin 2 nucleic acid molecule encodes a polypeptide having substantially identity to the amino acid sequence of SEQ ID NOs: 2 or 4. The nucleic acid molecule can include a sequence substantially identical to the nucleic acid sequence of SEQ ID NOs: 1 or 3. Preferably the nucleic acid molecule includes or consists of a sequence identical to the sequence of SEQ ID NOs: 1 or 3.

Additional useful lipocalin 2 compounds include any peptidyl or non-peptidyl compound that is a lipocalin 2 analog and has or induces lipocalin 2 biological activity; any peptidyl or non-peptidyl compound that binds to a lipocalin 2 receptor (e.g., 24p3R in mouse cells; see Devireddy et al., Cell 123:1293-1305, 2005); any peptidyl or non-peptidyl lipocalin 2 receptor agonists, including but not limited to agonistic antibodies; any compound known to stimulate or increase blood serum levels of lipocalin 2 polypeptides or increase the biological activity of lipocalin 2 polypeptides; any compound known to decrease the expression or biological activity of a lipocalin 2 inhibitor (e.g., an inhibitor that blocks binding to a siderophore or a lipocalin 2 receptor); and any compound that mimics lipocalin 2 effects on reducing raf, MEK, or ERK1/2 phosphorylation and/or biological activity, reversing EMT, reducing VEGF expression or biological activity, or inducing thrombospondin 1 expression or biological activity.

Preferred lipocalin 2 polypeptides, fragments or derivatives thereof, or non-peptidyl compounds will have at least 25%, 30%, 40%, 50%, 60%, 70%, 80%, 90%, 95%, 96%, 97%, 98%, 99%, or more lipocalin 2 biological activity. Non-limiting examples of lipocalin 2 biological activity include siderophore or iron-siderophore binding; reversal of EMT, as described herein, lipocalin 2 receptor binding (Devireddy et al., Cell 123:1293-1305, 2005), inhibition of ras-MAPK signaling pathway, reduction of E-cadherin phosphorylation, induction of E-cadherin expression or biological activity, induction of E-cadherin degradation, reduction of VEGF expression, induction of thrombospondin 1 expression, retinol transport, cryptic coloration, olfaction, pheromone transport, prostaglandin synthesis, and apopotosis (see Akerstrom et al., Biochim. Biophy. Acta 1482:1-8, 2000; and Flower et al., Biochem. J. 318:1-14, 1996). Assays for lipocalin 2 biological activity include assays for siderophore binding, iron transport, iron uptake (e.g., analysis of expression of ferritin protein levels and colorimetric determination of intracellular iron), and receptor binding, as described in Hanai et al., supra, Mori et al., supra, Li et al., supra, Yang et al., supra, and Devireddy et al., supra), and apoptotic assays known in the art. Preferably, the lipocalin 2 compound has siderophore or iron-siderophore binding activity and can transport iron and can reverse EMT by at least 25%, 30%, 40%, 50%, 60%, 70%, 80%, 90%, 95%, 96%, 97%, 98%, 99%, or more.

In preferred embodiments, the method further includes administering a siderophore to the subject. Non-limiting examples of siderophores are bacterial catecholate-type ferric siderophores, enterochelin, carboxymycobactin, aminochelin, desferrioxamine, aerobactin, arthrobactin, schizokinen, foroxymithine, pseudobactins, neoenactin, photobactin, ferrichrome, hemin, achromobactin, achromobactin, and rhizobactin (see U.S. Application Publication Number 20050261191). The siderophore can be administered alone, or in a pre-formed complex with lipocalin 2 and/or iron. The method can also include administering iron or an iron replacement therapy to the subject with or without the siderophore. For example, the method can include administering lipocalin 2 and iron; lipocalin and iron in a pre-formed complex; lipocalin, a siderophore and iron; or lipocalin, a siderophore, and iron in a pre-formed complex. Preferred iron replacements include ferrous sulfate and ferrous fumarate or dextran-iron for IV use and these can be administered orally or intravenously, as needed.

In yet another aspect, the invention features a method of diagnosing metastatic disease or a propensity to develop a metastatic disease in a subject having or at risk of having cancer, that includes (a) determining the level of a lipocalin 2 polypeptide, nucleic acid molecule, or fragments thereof, in a sample from the subject; and (b) comparing the level in (a) to a normal reference level of lipocalin 2 polypeptide, nucleic acid molecule, or fragment thereof; wherein an alteration in the subject levels relative to the normal reference level is diagnostic of a metastatic disease or a propensity to develop a metastatic disease in the subject. In preferred embodiments, the alteration is a decrease (e.g., 10%, 20%, 30%, 40%, 50%, 60%, 70%, 80%, 90%, or more).

The lipocalin 2 polypeptide can be measured using an immunological assay, enzymatic assay, or colorimetric assay. The sample can be a bodily fluid, tissue, or cell from the subject.

In yet another aspect, the invention features a method of monitoring the metastatic health of a subject having or at risk of having cancer, that includes the steps of (a) determining the level of a lipocalin 2 polypeptide, nucleic acid molecule, or fragments thereof, in a sample from the subject; and (b) comparing the level in (a) to a reference level of lipocalin 2, polypeptide, nucleic acid molecule, or fragments thereof; wherein an alteration in the subject levels relative to the reference level is an indicator of a change in the metastatic health of the subject. In preferred embodiments, the reference level is from a prior sample from the subject. The lipocalin 2 polypeptide can be measured using an immunological assay, enzymatic assay, or colorimetric assay. The sample can be a bodily fluid, tissue, or cell from the subject. In one example, this method is used to monitor a subject during treatment for a metastatic disease.

In another aspect, the invention features a kit for the diagnosis of a metastatic disease or the propensity to develop metastatic disease in a subject. The kit includes a lipocalin 2 binding protein (e.g., an antibody, or an antigen binding fragment thereof) and instructions for the use of the lipocalin 2 binding protein for the diagnosis of a metastatic disease or the propensity to develop metastatic disease.

In yet another aspect, the invention features a method of identifying a compound for the treatment of a metastatic disease. This method includes (a) contacting a cell that expresses lipocalin 2 polypeptide with a candidate compound, and (b) comparing the level of expression or biological activity of the lipocalin 2 polypeptide in the cell contacted by the compound with the level of expression in a control cell not contacted by the candidate compound. In this method, an alteration (e.g., an increase) in expression or biological activity of the lipocalin 2 polypeptide in said cell contacted by said compound identifies the candidate compound as a compound for the treatment of the metastatic disease.

In yet another aspect, the invention features a method of identifying a compound for the treatment of a metastatic disease. This method includes contacting a cell that expresses a lipocalin 2 nucleic acid molecule with a candidate compound, and comparing the level of expression or biological activity of the lipocalin 2 nucleic acid in the cell contacted by the compound with the level of expression in a control cell not contacted by the candidate compound, wherein an alteration in the expression or biological activity of the lipocalin 2 nucleic acid molecule in the cell contacted by the compound identifies the candidate compound as a compound for the treatment of a metastatic disease. In preferred embodiments, the alteration is an increase in the expression (e.g., an alteration in transcription or translation) or an increase in the biological activity of the lipocalin 2 nucleic acid molecule.

For the purpose of the present invention, the following abbreviations and terms are defined below.

By “alteration” is meant a change (increase or decrease) in the expression levels of a lipocalin 2 nucleic acid or polypeptide as detected by standard art known methods such as those described below. As used herein, an alteration includes a 10% change in expression levels, preferably a 25% change, more preferably a 40%, 50%, 60%, 70%, 80%, 90%, 95%, 96%, 97%, 98%, 99%, or greater change in expression levels. “Alteration” can also indicate a change (increase or decrease) in the biological activity of a lipocalin 2 nucleic acid or polypeptide. As used herein, an alteration includes a 10% change in biological activity, preferably a 25% change, more preferably a 40%, 50%, 60%, 70%, 80%, 90%, 95%, 96%, 97%, 98%, 99%, or greater change in biological activity. Examples of biological activity for lipocalin 2 polypeptides are described below.

By “angiogenesis” is meant the formation of new blood vessels and/or the increase in the volume, diameter, length, or permeability of existing blood vessels, such as blood vessels in a tumor or between a tumor and surrounding tissue. Angiogenesis is associated with a variety of neoplastic and non-neoplastic disorders.

By “angiogenic disorder” is meant a disease associated with excessive or insufficient blood vessel growth, an abnormal blood vessel network, and/or abnormal blood vessel remodeling. For example, insufficient vascular growth can lead to decreased levels of oxygen and nutrients, which are required for cell survival. Angiogenesis, in addition to being critical in metastases formation, also contributes to tumor growth. For any tumors, primary and metastatic, to grow beyond a few millimeters in diameter requires angiogenesis.

By “anti-fibrotic agent” is meant any agent, which can reduce or inhibit the production of extracellular matrix components including, but not limited to, fibronectin, proteoglycan, collagen, and elastin. Examples of anti-fibrotic agents are described herein and include antagonists of TGFβ and CTGF.

By “anti-cancer therapy” is meant any therapy intended to prevent, slow arrest or reverse the growth of a cancer or a cancer metastases. Generally, an anti-cancer therapy will reduce or reverse any of the characteristics that define the cancer cell (see Hanahan et al., Cell 100:57-50, 2000. Most cancer therapies target the cancer cell by slowing, arresting, reversing, decreasing the invasive capabilities, or decreasing the ability of the cell to survive the growth of a cancer cell. Additional anti-cancer therapies can target non-cancer cells including immune cells, endothelial cells, fibroblasts, immune and inflammatory cells or the extracellular matrix in the tumor microenvironment. Anti-cancer therapies include, without limitation, surgery, radiation therapy (radiotherapy), biotherapy, immunotherapy, chemotherapy, or a combination of these therapies.

By “chemotherapy” is meant the use of a chemical agent to destroy a cancer cell, or to slow, arrest, or reverse the growth of a cancer cell.

By “chemotherapeutic agent” is meant a chemical that may be used to destroy a cancer cell, or to slow, arrest, or reverse the growth of a cancer cell. Chemotherapeutic agents include, without limitation, asparaginase, bleomycin, busulfan carmustine (commonly referred to as BCNU), chlorambucil, cladribine (commonly referred to as 2-CdA), CPT11, cyclophosphamide, cytarabine (commonly referred to as Ara-C), dacarbazine, daunorubicin, dexamethasone, doxorubicin (commonly referred to as Adriamycin), etoposide, fludarabine, 5-fluorouracil (commonly referred to as 5FU), hydroxyurea, idarubicin, ifosfamide, interferon-α (native or recombinant), levamisole, lomustine (commonly referred to as CCNU), mechlorethamine (commonly referred to as nitrogen mustard), melphalan, mercaptopurine, methotrexate, mitomycin, mitoxantrone, paclitaxel, pentostatin, prednisone, procarbazine, tamoxifen, taxol-related compounds, 6-thiogaunine, topotecan, vinblastine, and vincristine.

By “compound” is meant any small molecule chemical compound, antibody, nucleic acid molecule, or polypeptide, or fragments thereof.

By “effective amount” is meant an amount sufficient to prevent or reduce any of the disorders of the invention including cancer, metastatic disease, angiogenic disorders, or fibrotic disorders or any symptom associated with the disorder. It will be appreciated that there will be many ways known in the art to determine the therapeutic amount for a given application. For example, the pharmacological methods for dosage determination may be used in the therapeutic context.

By “efficacy” is meant the effectiveness of a particular treatment regime. Efficacy in anti-cancer or anti-cancer metastasis treatment regimes can be measured based on such non-limiting characteristics as, for example, by reduction or inhibition of tumor growth or tumor mass, or reduction of metastatic lesions.

By “epithelial to mesenchymal transition” or “EMT” is meant the change in phenotype of an epithelial cell, from a phenotype that is polarized and that grows appositionally to a phenotype that is mobile, more-fibroblast like and invasive. Molecular markers of EMT include the presence of alpha smooth muscle actin, the presence of vimentin, or the loss of E-cadherin expression. Any or all of these can be measured at the protein level or the nucleic acid level and can be used as a marker of EMT.

By “expression” is meant the detection of a gene or polypeptide by standard art known methods. For example, polypeptide expression is often detected by western blotting, DNA expression is often detected by Southern blotting or polymerase chain reaction (PCR), and RNA expression is often detected by Northern blotting, PCR, or RNAse protection assays.

By “fibrosis” is meant the formation of excessive fibrous tissue, as in a reparative or reactive process. One of the principle fibrous tissues formed in excess during the course of fibrosis is collagen. Fibrosis can occur in response to physical or chemical injury to a tissue, or can be the result of abnormal tissue response and/or physiology, such as occurs in some disease states. A subject with a fibrotic condition refers to, but is not limited to, subjects afflicted with fibrosis of an internal organ, subjects afflicted with a dermal fibrosing disorder, and subjects afflicted with fibrotic conditions of the eye. Fibrosis of internal organs (e.g., liver, lung, kidney, heart blood vessels, and gastrointestinal tract), occurs in disorders such as pulmonary fibrosis, myelofibrosis, liver cirrhosis, mesangial proliferative glomerulonephritis, crescentic glomerulonephritis, diabetic nephropathy, renal interstitial fibrosis, renal fibrosis in patients receiving cyclosporin, and HIV associated nephropathy. Dermal fibrosing disorders include, but are not limited to, scleroderma, morphea, keloids, hypertrophic scars, familial cutaneous collagenoma, and connective tissue nevi of the collagen type. Fibrotic conditions of the eye include conditions such as diabetic retinopathy, postsurgical scarring (for example, after glaucoma filtering surgery and after cross-eye surgery), and proliferative vitreoretinopathy. Additional fibrotic conditions which may be treated by the methods of the present invention include rheumatoid arthritis, diseases associated with prolonged joint pain and deteriorated joints, progressive systemic sclerosis, polymyositis, dennatomyositis, eosinophilic fascitis, morphea, Raynaud's syndrome, and nasal polyposis. In addition, fibrotic conditions which may be treated by the methods of present invention also include overproduction of scarring in patients who are known to form keloids or hypertrophic scars, scarring or overproduction of scarring during healing of various types of wounds including surgical incisions, surgical abdominal wounds, and traumatic lacerations, scarring and reclosing of arteries following coronary angioplasty, excess scar or fibrous tissue formation associated with cardiac fibrosis after infarction and in hypersensitive vasculopathy.

By “fragment” is meant a portion of a polypeptide or nucleic acid molecule that contains, preferably, at least 10%, 20%, 30%, 40%, 50%, 60%, 70%, 80%, 90%, 95%, or more of the entire length of the reference nucleic acid molecule or polypeptide. A fragment may contain 10, 20, 30, 40, 50, 60, 70, 80, 90, or 100, 200, 300, 400, 500, 600, or more nucleotides or 10, 20, 30, 40, 50, 60, 70, 80, 90, 100, 120, 140, 160, 180, 190, 198 amino acids or more. Preferred fragments of lipocalin 2 will have lipocalin 2 biological activity and may include, for example, the lipocalin 2 receptor binding domain or the iron siderophore binding domain (see Holmes et al., Structure 13:29-41, 2005) for characterization of the enterochelin binding domain of lipocalin 2). Non-limiting examples of residues that are important for the binding of siderophores include R81, K125, and K134 and preferred fragments of lipocalin 2 include these residues.

By “heterologous” is meant any two or more nucleic acid or polypeptide sequences that are not normally found in the same relationship to each other in nature. For instance, the nucleic acid is typically recombinantly produced, having two or more sequences, e.g., from unrelated genes arranged to make a new functional nucleic acid, e.g., a promoter from one source and a coding region from another source. Similarly, a heterologous polypeptide will often refer to two or more subsequences that are not found in the same relationship to each other in nature (e.g., a fusion protein).

By a “high dosage” is meant at least 5% (e.g., at least 10%, 20%, 50%, 100%, 200%, or even 300%) more than the highest standard recommended dosage of lipocalin 2 compound formulated for a given route of administration for treatment of a disease or condition.

By “homologous” is meant any gene or polypeptide sequence that bears at least 30% homology, more preferably 40%, 50%, 60%, 70%, 80%, and most preferably 90%, 95%, 96%, 97%, 98%, 99%, or more homology to a known gene or polypeptide sequence over the length of the comparison sequence. A “homologous” polypeptide can also have at least one biological activity of the comparison polypeptide. For polypeptides, the length of comparison sequences will generally be at least 16 amino acids, preferably at least 20 amino acids, more preferably at least 30, 40, 50, 60, 70, 80, 90, 100, 120, 140, 160, 180, 190, 198 amino acids or more. For nucleic acids, the length of comparison sequences will generally be at least 50 nucleotides, preferably at least 60, 70, 80, 90, 100, 150, 200, 250, 300, 350, 400, 450, 500, 550, 600, or more.

“Homology” can also refer to a substantial similarity between an epitope used to generate antibodies and the protein or fragment thereof to which the antibodies are directed. In this case, homology refers to a similarity sufficient to elicit the production of antibodies that can specifically recognize the protein or polypeptide.

By “kinase activity” is meant the ability to catalyze the transfer a phosphate group from adenosine triphosphate (ATP) to a residue (e.g., tyrosine, threonine, serine) on a substrate polypeptide or protein.

By “lipocalin 2” or “lipocalin 2 compound” is meant a polypeptide, or a nucleic acid sequence that encodes it, or fragments or derivatives thereof, that is substantially identical or homologous to or encodes any of the following amino acid sequences: SEQ ID NOS: 2 (human) and 4 (mouse), GenBank Accession Numbers NM_{—}005564, BU174414, BC033089, P80188, P30152, NP032517, CAA67099, AAB35994, P11672, and CAA58127, and that has lipocalin 2 biological activity (e.g., siderophore binding, iron transport, or lipocalin 2 receptor binding) as described below. Lipocalin 2 nucleic acid molecules encode a lipocalin 2 polypeptide and preferably have substantial identity to the nucleic acid sequence of SED ID NO: 1 (human) or 3 (mouse). Lipocalin 2 can also include fragments, derivatives, or analogs of lipocalin 2, including non-peptidyl small molecule compounds, that have iron-siderophore binding properties and that retain at least 25%, 30%, 40%, 50%, 60%, 70%, 80%, 90%, 95%, 96%, 97%, 98%, 99%, or more lipocalin 2 biological activity. The lipocalin 2 polypeptides may be isolated from a variety of sources, such as from mammalian tissue or cells or from another source, or prepared by recombinant or synthetic methods. The term “lipocalin 2” also encompasses modifications to the polypeptide, fragments, derivatives, analogs, and variants of the lipocalin 2 polypeptide. Lipocalin 2 is also known as “siderocalin,” “Ngal,” “24p3,” “uterocalin,” and “neu related lipocalin,” all of which are encompassed by the term lipocalin 2.

By “lipocalin 2 biological activity” is meant the any of the following activities: siderophore or iron-siderophore binding; lipocalin 2 receptor binding (Devireddy et al., Cell 123:1293-1305, 2005), inhibition of ras-MAPK signaling pathway, reduction of E-cadherin phosphorylation, induction of E-cadherin expression, induction of E-cadherin degradation, retinol transport, cryptic coloration, olfaction, pheromone transport, prostaglandin synthesis, and apopotosis (see Akerstrom et al., Biochem. Biophy. Acta 1482:1-8, 2000; and Flower et al., Biochem. J. 318:1-14, 1996). Assays for lipocalin 2 biological activity include assays for siderophore binding, iron binding, iron uptake (e.g., analysis of expression of ferritin protein levels and calorimetric determination of intracellular iron), and receptor binding, as described in Hanai et al., supra, Mori et al., supra, Li et al., supra, Yang et al., supra, and Devireddy et al., supra), and apoptotic assays known in the art. Additional examples of assays for biological activity for lipocalin 2 are described herein, including, for example, reversal of EMT, and VEGF downregulation.

By a “low dosage” is meant at least 5% less (e.g., at least 10%, 20%, 50%, 80%, 90%, or even 95%) than the lowest standard recommended dosage of a lipocalin 2 compound formulated for a given route of administration for treatment of a disease or condition.

By “metastasis” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant.

By “metastatic disease,” “metastases,” and “metastatic lesion” are meant a group of cells which have migrated to a site distant relative to the primary tumor. “Non-metastatic” refers to tumor cells, e.g., human cancer cells, that are unable to establish secondary tumor lesions distant to the primary tumor. Although not often the case, metastatic disease can occur when no primary tumor has been detected. The cells in a metastatic tumor resemble those in the primary tumor. Metastasis or metastatic disease can be diagnosed in a variety of ways that are known in the art. Generally, metastatic disease is diagnosed using radiological methods such as Xray, CT scan, ultrasound, or MRI. PET scan can also be used. Additional techniques such as Circulating Tumor Cell analysis (CTC) can be used to determine the number of epithelial cells present in a sample of bodily fluid (e.g., blood). For example, in normal patients there are very few if any (typically less than 1) epithelial cells/ml of blood. If a patient is found to have a relatively higher CTC count (e.g., 2, 3, 5, 10, 15, 20, 25, 50, 100, 250, 500, 1000, or more) epithelial cells, this is considered an indicator of metastatic disease and the disease can then be confirmed using additional methods described herein. Such CTC kits are commercially available and include CellSearch™ Epithelial Cell Kit and CellSpotter™ (Veridex, Warren, N.J.). If needed, a biopsy can be performed, either in conjunction with the radiological methods or separately, and the tissue can be examined for molecular markers of the metastatic disease either at the protein, DNA, or RNA level. In a biopsy, metastases are typically diagnosed by the presence of cells, or molecular markers, that are not normally found in the part of the body from which the tissue sample was taken. For example, if a tissue sample taken from a tumor in the lung contains cells that look like breast cells, the doctor determines that the lung tumor is a secondary tumor to the primary breast cancer. The molecular markers can be markers of cancer or metastatic disease (e.g., p53, VHL, or BRCA mutations), markers of the primary tumor, or markers of the primary tumor cell type (e.g., breast cells found in the lung in the above example) or any combination of these. Identification of a metastasis and determination can include the use of several techniques, such as immunohistochemistry, FISH (fluorescent in situ hybridization), gene array profiling, RNA analysis by RT-PCR, and others. It should be noted that metastases may not have an identical profile to the cells of the primary tumor but will have a profile that is substantially more similar to the profile of the primary tumor than to the cells at the metastatic site in question. For example, if a lung biopsy is obtained and analyzed by gene expression profiling, the profile may be 90% identical to the profile obtained from the breast cancer biopsy and only 50% identical to the profile of a lung cell taken from the area surrounding the metastatic site.

By “metric” is meant a measure. A metric may be used, for example, to compare the levels of a polypeptide or nucleic acid molecule of interest. Exemplary metrics include, but are not limited to, mathematical formulas or algorithms, such as ratios. The metric to be used is that which best discriminates between levels of lipocalin 2 polypeptide in a subject having cancer and a normal reference subject. Depending on the metric that is used, the diagnostic indicator of a metastatic disease may be significantly above or below a reference value (e.g., from a control subject not having cancer).

By “pharmaceutically acceptable carrier” is meant a carrier that is physiologically acceptable to the treated mammal while retaining the therapeutic properties of the compound with which it is administered. One exemplary pharmaceutically acceptable carrier substance is physiological saline. Other physiologically acceptable carriers and their formulations are known to one skilled in the art and described, for example, in Remington's Pharmaceutical Sciences, (20^{th} edition), ed. A. Gennaro, 2000, Lippincott, Williams & Wilkins, Philadelphia, Pa.

By “preventing” is meant prophylactic treatment of a subject who is not yet ill, but who is susceptible to, or otherwise at risk of, developing a particular disease. Preferably a subject is determined to be at risk of developing metastasis, angiogenic disorders, or fibrotic disorders using the diagnostic methods known in the art or described herein. For example, when used with relation to metastatic disease, “preventing” can refer to the preclusion of metastatic disease occurrence in a patient diagnosed with a primary cancer. Specifically the preventive measures are used to prevent a primary cancer, that is invasive or prone to metastatic disease, from metastasizing, where the cancer would otherwise be predicted, based on statistic or clinical characteristics of the cancer that are known to be associated with metastatic disease, to metastasize.

By “primary tumor” or “primary cancer” is meant the original cancer and not a metastatic lesion located in another tissue or organ in the subject's body.

By “proliferation” is meant an increase in cell number, i.e., by mitosis of the cells. As used herein proliferation does not refer to cancer cell growth.

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

By “protein,” “polypeptide,” or “polypeptide fragment” is meant any chain of more than two amino acids, regardless of post-translational modification (e.g., glycosylation or phosphorylation), constituting all or part of a naturally occurring polypeptide or peptide, or constituting a non-naturally occurring polypeptide or peptide.

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one time administration and typical dosages range from 10 to 200 units (Grays) per day.

By “ras-MAPK pathway” is meant any cell-signaling pathway that is initiated by a signaling event from a ras family member and can include activation of any of the family kinases known as the MAPKs that play an essential role in signal transduction pathways modulating gene expression in the nucleus in response to changes in the cellular environment. The cellular ras genes encode proteins of 21 kDa that bind guanine nucleotides and cycle between an activated or inactivated form, respectively ras-GTP and ras-GDP. The best characterized ras signal transduction pathway is the Raf/MEK/ERK MAPK cascade. Active Ras-GTP forms a high-affinity complex with the serine-threonine protein kinase protein Raf which is then recruited from the cytosol to the plasma membrane leading to its activation.

By “reduce or inhibit” is meant the ability to cause an overall decrease preferably of 20% or greater, more preferably of 50% or greater, and most preferably of 75%, 85%, 90%, 95%, or greater. Reduce or inhibit can refer to the symptoms of the disorder being treated, the presence or size of metastases, the size of the primary tumor, the size or number of the blood vessels in angiogenic disorders, or the size or extent of scarring in fibrotic disorders. For diagnostic or monitoring applications, reduce or inhibit can refer to the level of protein or nucleic acid, detected by the aforementioned assays (see “expression”).

By “reference sample” is meant any sample, standard, or level that is used for comparison purposes. A “normal reference sample” can be a prior sample taken from the same subject, a sample from a subject not having cancer, a subject that is diagnosed with cancer but not a metastatic disease, a subject that has been treated for either cancer, metastatic disease, or both, a subject that has a benign tumor, or a sample of a purified reference lipocalin 2 polypeptide at a known normal concentration. By “reference standard or level” is meant a value or number derived from a reference sample. A normal reference standard or level can be a value or number derived from a normal subject that is matched to the sample subject by at least one of the following criteria: age, weight, disease stage, overall health, prior diagnosis of cancer, location of primary tumor or metastasis, and a family history of cancer or metastatic disease. A “positive reference” sample, standard or value is a sample or value or number derived from a subject that is known to have a metastatic disorder, that is matched to the sample subject by at least one of the following criteria: age, weight, disease stage, overall health, prior diagnosis of cancer, location of primary tumor or metastasis, and a family history of cancer or metastatic disease.

By “sample” is meant a bodily fluid (e.g., urine, blood, serum, plasma, or cerebrospinal fluid), tissue, or cell in which the lipocalin 2 polypeptide or nucleic acid molecule is normally detectable.

By “subject” is meant a mammal, including, but not limited to, a human or non-human mammal, such as a bovine, equine, canine, ovine, or feline.

By “substantially identical” is meant a nucleic acid or amino acid sequence that, when optimally aligned, for example using the methods described below, share at least 60%, 65%, 70%, 75%, 80%, 85%, 90%, 95%, 96%, 97%, 98%, 99%, or 100% sequence identity with a second nucleic acid or amino acid sequence, e.g., a lipocalin 2 sequence. “Substantial identity” may be used to refer to various types and lengths of sequence, such as full-length sequence, epitopes or immunogenic peptides, functional domains, coding and/or regulatory sequences, exons, introns, promoters, and genomic sequences. Percent identity between two polypeptides or nucleic acid sequences is determined in various ways that are within the skill in the art, for instance, using publicly available computer software such as Smith Waterman Alignment (Smith and Waterman J. Mol. Biol. 147:195-7, 1981); “BestFit” (Smith and Waterman, Advances in Applied Mathematics, 482-489, 1981) as incorporated into GeneMatcher Plus™, Schwarz and Dayhof “Atlas of Protein Sequence and Structure,” Dayhof, M. O., Ed pp 353-358, 1979; BLAST program (Basic Local Alignment Search Tool; (Altschul, S. F., W. Gish, et al., J. Mol. Biol. 215: 403-410, 1990), BLAST-2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the length of the sequences being compared. In general, for proteins, the length of comparison sequences will be at least 10 amino acids, preferably 20, 30, 40, 50, 60, 70, 80, 90, 100, 110, 120, 130, 140, 150, 160, 170, 180, 190, or at least 198 amino acids or more. For nucleic acids, the length of comparison sequences will generally be at least 25, 50, 100, 125, 150, 200, 250, 300, 350, 400, 450, 500, 550, or at least 600 nucleotides or more. It is understood that for the purposes of determining sequence identity when comparing a DNA sequence to an RNA sequence, a thymine nucleotide is equivalent to a uracil nucleotide. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

By “treating” is meant administering a compound or a pharmaceutical composition for prophylactic and/or therapeutic purposes or administering treatment to a subject already suffering from a disease to improve the subject's condition or to a subject who is at risk of developing a disease. By “treating cancer,” “treating a metastatic disease,” “treating an angiogenic disorder,” or “treating a fibrotic disorder” is meant that the disease and the symptoms associated with the disease are alleviated, reduced, cured, or placed in a state of remission. More specifically, when lipocalin 2, or fragments or derivatives thereof, are used to treat a subject with a tumor, it is generally provided in a therapeutically effective amount to achieve any one or more of the following: inhibited tumor growth, reduction in tumor mass, or reduction in tumor such that there is no detectable disease, slowing or preventing an increase in the size of a tumor (as assessed by e.g., radiological imaging, biological fluid analysis, cytogenetics, fluorescence in situ hybridization, immunocytochemistiy, colony assays, multiparameter flow cytometry, or polymerase chain reaction). For example, a therapeutic amount can cause a qualitative or quantitative reduction (e.g., by at least 10%, 20%, 30%, 40%, 50%, 60%, 70%, 80%, 90%, 95%, or more) in the tumor or metastases size or reduce or prevent metastatic growth. Preferably, when lipocalin 2, or fragments or derivatives thereof, are used to treat a subject with a metastatic cancer, it is generally provided in a therapeutically effective amount sufficient to prevent metastasis or to reduce metastatic disease or metastatic lesions, to inhibit development of new metastatic lesions after treatment has started, to increase the disease-free survival time between the disappearance of a tumor, or a metastases, and its reappearance, to prevent an initial or subsequent occurrence of a tumor or metastases, or to reduce any adverse symptom associated with a tumor or a metastases. In one preferred embodiment, the percent of cancerous or metastatic cells surviving the treatment is at least 20, 40, 60, 80, or 100% lower than the initial number of cancerous or metastatic cells, as measured using any standard assay. Preferably, the decrease in the number of cancerous or metastatic cells induced by administration of a therapy of the invention is at least 2, 5, 10, 20, or 50-fold greater than the decrease in the number of non-cancerous or non-metastatic cells. In yet another preferred embodiment, the number of cancerous or metastatic cells present after administration of a therapy is at least 2, 5, 10, 20, or 50-fold lower than the number of cancerous or metastatic cells present after administration of a vehicle control. Preferably, the methods of the present invention result in a decrease of 20, 40, 60, 80, or 100% in the size of a primary or metastatic tumor as determined using standard methods. Preferably, the cancer does not reappear or reappears after at least 2, 5, 10, 15, or 20 years. In another preferred embodiment, the length of time a patient survives after being diagnosed with cancer and treated with a therapy of the invention is at least 20, 40, 60, 80, 100, 200, or even 500% greater than (i) the average amount of time an untreated patient survives or (ii) the average amount of time a patient treated with another therapy survives.

When lipocalin 2, or fragments or derivatives thereof, is used to treat a subject with an angiogenic disorder, it is generally provided in a therapeutically effective amount to achieve any one or more of the following: a reduction or inhibition in the formation of new blood vessels and/or modulating the volume, diameter, length, permeability, or number of existing blood vessels. In preferred embodiments, an initial or subsequent occurrence of an angiogenesis related disorder is prevented or an adverse symptom associated with an angiogenesis related disorder is reduced. Preferably, the methods of the present invention result in a reduction or inhibition of 20, 40, 60, 80, or even 100% in the volume, diameter, length, permeability, and/or number of blood vessels as determined using standard methods. Preferably, at least 20, 40, 60, 80, 90, or 95% of the treated subjects have a complete remission in which all evidence of the disease disappears. In another preferred embodiment, the length of time a patient survives after being diagnosed with an angiogenesis related disease and treated with a therapy of the invention is at least 20, 40, 60, 80, 100, 200, or even 500% greater than (i) the average amount of time an untreated patient survives or (ii) the average amount of time a patient treated with another therapy survives.

When lipocalin 2, or fragments or derivatives thereof, is used to treat a subject with a fibrotic disorder, it is generally provided in a therapeutically effective amount to achieve any one or more of the following: prevent or reduce scarring or overproduction of scarring (for example, scarring in patients who are known to form keloids or hypertrophic scars, scarring or overproduction of scarring during healing of various types of wounds including surgical incisions, surgical abdominal wounds and traumatic lacerations, scarring and reclosing of arteries following coronary angioplasty, and excess scar or fibrous tissue formation associated with such non-limiting conditions such as liver fibrosis (including cirrhosis), lung fibrosis (e.g., silicosis, asbestosis), kidney fibrosis (including diabetic nephropathy, chronic renal failure, and glomerulosclerosis), sclerodoma, bone marrow fibrosis, bone fibrosis, prevent or reduce excess scar or fibrous tissue formation, and prevent or reduce contracture or adhesion formation. Preferably, the methods of the present invention result in reduction of at least 20%, 40%, 60%, 80%, or 100% in the volume, diameter, or length of the scarring, fibrosis, contracture, or adhesion formation as determined using standard methods. Efficacy in anti-fibrotic treatment regimes can be measured based on such non-limiting characteristics as, for example, by the stabilization, reversal, slowing or delay progression of a fibrotic condition in accordance with clinically acceptable standards for disorders to be treated or for cosmetic purposes. Detection and measurement of indicators of efficacy may be measured by a number of available diagnostic tools, including, for example, by physical examination, blood tests, organ function tests, X-rays, MRI, biopsy, and CT scan. (See Fibrosis Applications, below.)

By “tumor” or “cancer” is meant both benign and malignant growths of cancer. Thus, the term “cancer,” unless otherwise stated, can include both benign and malignant growths. Preferably, the tumor is malignant. The tumor can be a non-solid tumor (a tumor that grows within the blood stream) or a solid tumor, which refers to one that grows in an anatomical site outside the bloodstream (in contrast, for example, to blood-borne tumors, such as lymphomas and leukemia) and requires the formation of small blood vessels and capillaries to supply nutrients, etc., to the growing tumor mass. Solid tumors can be separated into those of epithelial cell origin and those of non-epithelial cell origin. Examples of epithelial cell solid tumors include tumors of the gastrointestinal tract, colon, breast, prostate, lung, kidney, liver, pancreas, ovary, head and neck, oral cavity, stomach, duodenum, small intestine, large intestine, anus, gall bladder, labium, nasopharynx, skin, uterus, male genital organ, urinary organ, bladder, and skin. Solid tumors of non-epithelial origin include sarcomas, brain tumors, and bone tumors.

By “vector” is meant a DNA molecule, usually derived from a plasmid or bacteriophage, into which fragments of DNA may be inserted or cloned. A recombinant vector will contain one or more unique restriction sites, and may be capable of autonomous replication in a defined host or vehicle organism such that the cloned sequence is reproducible. A vector contains a promoter operably linked to a gene or coding region such that, upon transfection into a recipient cell, an RNA is expressed.

Other features and advantages of the invention will be apparent from the following Detailed Description, the drawings, and the claims.

### BRIEF DESCRIPTION OF THE DRAWINGS

FIG. 1A shows the nucleic acid sequence of human lipocalin 2 (SEQ ID NO: 1). FIG. 1B shows the amino acid sequence of human lipocalin 2 (SEQ ID NO: 2). FIG. 1C shows the nucleic acid sequence of mouse lipocalin 2 (SEQ ID NO: 3). FIG. 1D shows the amino acid sequence of mouse lipocalin 2 (SEQ ID NO: 4).

FIG. 2A shows phase contrast (upper) and fluorescent (lower) images for E-cadherin by confocal microscopy. FIG. 2B shows a photograph of western blots of 4T1-EV (EV), 4T1-ras (R), and 4T1-ras cells expressing lipocalin 2 (RL) cells blotted with antibodies to E-cadherin, vimentin and GAPDH. FIG. 2C shows a photograph of Northern blots of E-cadherin and GAPDH RT-PCR analysis. FIG. 2D shows a photograph of a western blot depicting E-cadherin protein levels in R cells transiently transfected with lipocalin 2 pcDNA3.1. Transfected amounts of lipocalin 2-pcDNA3.1 were 0, 1, and 2 μg/well (lanes 1-3 respectively) and lane 4 (EV) represents 4T1-EV cells as a control. Total amount of transfected cDNA was equalized with the empty vector pcDNA3.1. FIG. 2E shows a photograph of a western blot depicting E-cadherin protein levels in R cells cultured with conditioned medium (CM) containing lipocalin 2 produced from 293T cells transfected with lipocalin 2-pcDNA3.1. Amount of media from lipocalin 2-transfected 293T cells was 0, 1, and 2 ml for lanes 1-3 respectively with total amount of media equalized by addition of media from empty-vector transfected 293T cells. Lane 4 (EV) represents EV cells as a control. GAPDH serves as a loading control.

FIG. 3 is a graph depicting the invasion migration of stable 4T1 clones using EV, R, and RL cells. Polycarbonate membranes of Transwells were coated with Matrigel® and cells were seeded. Sixteen hours later, cells were fixed, stained with Giemsa solution, and counted for each of the stable clones; EV, R, and RL.

FIG. 4A is a graph showing the effect of lipocalin 2 on 4T1 primary tumor growth. 4T1 clones (EV, R, and RL) were suspended in PBS and injected subcutaneously in the backs of Balb/c mice. Primary tumor size was calculated based upon measurements at 1, 2, and 3 weeks. FIG. 4B shows photographs of hematoxylin and eosin (H & E) stained tumor sections. White arrow in the middle shows muscle tissue into which tumor has invaded. FIG. 4C shows a western blot of lysate from primary tumors using EV, R, and RL cells. FIG. 4D shows Northern blots from RT-PCR analysis of primary tumors using EV, R, and RL cells. Top lane shows the expression of lipocalin 2 mRNA in the RL stable cell clone using primers directed against the HA tag in the lipocalin 2 cDNA. FIGS. 4E-F show graphs depicting the lung weight (FIG. 4E) and the number of metastatic nodules on the lung surface (FIG. 4F). FIG. 4G shows photographs of H & E stained lung sections.

FIG. 5 shows the effects of PI3K and MEK inhibitors on ras-induced epithelial to mesenchymal transition (EMT). Shown are photographs of the fluorescent images produced from E-cadherin staining in R cells by confocal microscopy. R cells (left panel) were incubated with the PI3K inhibitor (LY294002, 10 μM) (middle panel) and MEK inhibitor (U0126, 10 μM) (right panel). Below are western blots of E-cadherin and GAPDH for each condition.

FIG. 6A shows western blots using phosphospecific antibodies illustrating the effects of lipocalin 2 on phosphorylation state of ras-MAPK signaling molecules. FIG. 6B shows a graph depicting the ratio of renilla luciferase to sea-pansy luciferase using 4T1 clones (EV, R, and RL). The SRE-luciferase assay was performed after 48 h incubation in serum free DMEM and ratio of renilla luciferase to sea-pansy luciferase is shown on the ordinate. FIG. 6C shows phase contrast images of RL cells with 0, 200, and 400 multiplicity of infection (MOI) of MEK-DD adenovirus (right, middle, and right panel respectively). All images were taken at 24 hours after the final plating. FIG. 6D shows western blots of cell lysates 48 h after the final plating. FIG. 6E shows a graph depicting the ratio of renilla luciferase to sea-pansy luciferase using 4T1-EV cells with or without Lipo:Sid:Fe, SRE-luciferase.

FIGS. 7A-D demonstrate proteasome inhibitor effects on ras-induced EMT and effects of ras, lipocalin 2, and a MEK inhibitor on E-cadherin phosphorylation. FIG. 7A shows phase contrast images illustrating the morphology of R cells treated with proteasome inhibitor MG132 (0.5 nM) for 48 hours. FIG. 7B shows western blots of stable clones (EV, R, and RL) with or without proteasome inhibitor MG132 (48 hours). FIG. 7C shows western blots of Hakai protein expression levels in 4T1 clones. FIG. 7D shows western blots illustrating E-cadherin phosphorylation, protein level, and mRNA levels in EV, R, and RL cells and R cells treated with the MEK inhibitor (U0126).

FIG. 8A shows phase contrast images showing RL cells incubated with deferoxamine mesylate (DFO) for 48 hours. Below are western blots for E-cadherin and GAPDH. The DFO concentrations were 0, 2, and 5 μM (left, middle, and right panels or lanes, respectively). FIG. 8B shows western blots illustrating E-cadherin expression in R cells incubated with Lipo:Sid:Fe (lanes 7-8), Lipo:Sid (lanes 5-6), Lipo (lanes 3-4), or PBS (lanes 1-2). The protein concentrations were 15 μg/ml (lanes 4, 6, and 8) or 50 μg/ml (lanes 3, 5, and 7). FIG. 8C shows western blots depicting the effects of lipocalin 2 formulations on ERK phosphorylation. R cells at 50% confluency on 6-well plate were incubated in 0% serum including DMEM for 48 hours with PBS or lipocalin 2.

FIG. 9 shows a schematic summarizing the effect of lipocalin 2 on ras induced signaling. The schematic shows (1) that lipocalin 2 antagonizes ras signaling at a point upstream of raf activation in the ras-MAPK pathway, and (2) that activation of the ras-MAPK pathway leads to phosphorylation of E-cadherin due to the action of MEK or a downstream kinase.

FIG. 10A shows a western blot of R cells converted to an epithelial phenotype by lipocalin 2 transfection. FIG. 10B shows phase contrast images of R cells treated with various lipocalin 2 formulations in the same conditions as in FIG. 8C. Lipo:Sid:Fe, Lipo:Sid, and Lipo proteins were used at a concentration of 50 μg/ml.

FIG. 11 shows VEGF secretion from 4T1 cell clones. VEGF levels were determined by ELISA. VEGF secretion was stimulated approximately 10 fold by ras transformation (R cells) and downregulated (≈7.5 fold) by lipocalin 2 (RL cells).

FIG. 12 shows VEGF and TSP-1 expressions in each 4T1 primary tumor in vivo. 4T1 clones (EV, R and RL) were suspended in PBS and injected subcutaneously in the backs of Balb/c mice. 3 weeks later, primary tumor tissue was dissected, homogenized and the supernatant fluid was collected as total cell lysate. Western blot of total cell lysate from each primary tumor for the antigen is shown. GAPDH serves as a loading control. The E-cadherin, vimentin and GAPDH data is from Hanai et al., J. Biol. Chem. 280:13641-13647 (2005).

FIG. 13 shows VEGF induction in ras transformed cells is regulated by MEK and PI3K. Conditioned media from R cells (cultured in a 6-well plate) treated with the MEK inhibitor and the PI3K inhibitor (2 days of incubation) were analyzed by ELISA for VEGF concentration.

FIGS. 14A-14B are western blots for phospho-AKT (pAKT) showing the downregulation of ras-induced AKT phosphorylation, but not IGF-1 induced AKT phosphorylation, by lipocalin 2. FIG. 14A is a western blot for pAKT showing the lysate from each 4T1 clone (EV, R and RL). FIG. 14B is a western blot for pAKT showing the lysate from EV or RL cells treated with or without IGF-1. GAPDH serves as a loading control.

FIGS. 15A-15C show VEGF mRNA expression by RT-PCR for 4T1 cell clones and regulation by MEK and PI3K. FIG. 15A shows VEGF mRNA levels in EV, R, and RL cells. FIG. 15B shows VEGF mRNA levels in R cells treated with the MEK inhibitor and PI3K inhibitor at the indicated doses for 24 hours. GAPDH serves as a loading control. FIG. 15C shows the VEGF mRNA levels in RL cells infected with an adenovirus carrying the MEK dominant active form (MEK-DD), constitutively active AKT (CA-AKT), and a Lac-Z adenovirus at the indicated multiplicities (MOI). GAPDH serves as a loading control.

FIG. 16 is a graph showing the downregulation of VEGF secretion by lipocalin 2 in RL cells and the reversal with constitutively active MEK and AKT. Conditioned media from RL cells infected with constitutively active MEK and AKT adenovectors were analyzed by ELISAs for VEGF concentration.

FIGS. 17A-17C show the involvement of caveolin-1 in the MET-inducing and anti-angiogenic function of lipocalin 2. FIG. 17A shows western blot analysis of caveolin-1 expression in clones EV, R, and RL. FIG. 17B shows western blot analysis of RL cells infected with an adenovirus carrying the caveolin-1 antisense or a Lac-Z adenovirus at the indicated multiplicities (MOI). FIG. 17C shows western blot analysis of R cells infected with an adenovirus carrying caveolin-1 sense and a Lac-Z in the same condition as in FIG. 17B.

FIG. 18 shows a schematic diagram of the effects of lipocalin 2 on angiogenesis signaling pathways.

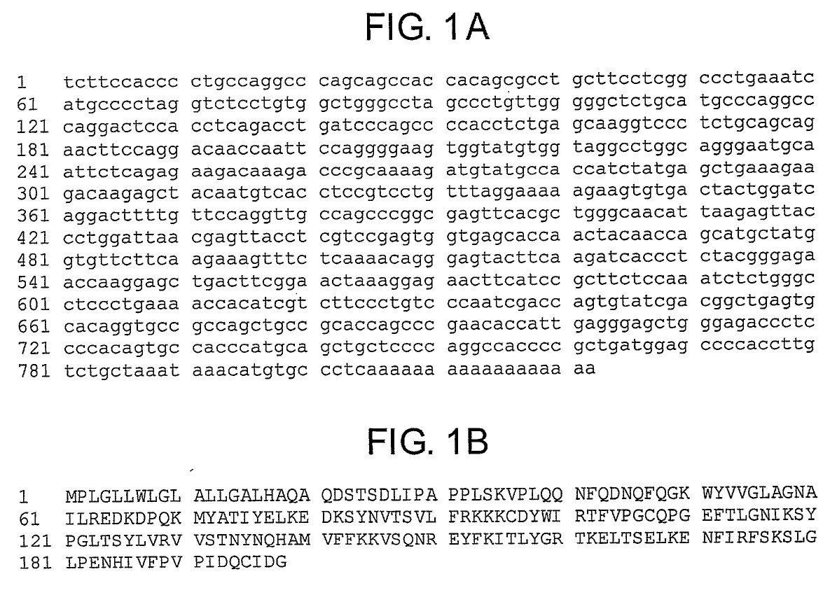

### DETAILED DESCRIPTION

We have discovered that lipocalin 2, an iron-siderophore binding protein reverses the transition of epithelial cells to mesenchymal cells (EMT), a process that is involved in metastasis, fibrosis, and angiogenesis. We have also discovered that lipocalin 2 increases E-cadherin expression, blocks VEGF production and induces thrombospondin expression. Furthermore, we have discovered that lipocalin 2 suppresses cell invasiveness in vitro and tumor growth and lung metastases in vivo. Thus, the present invention features the use of lipocalin 2, biologically active fragments or derivatives thereof, as a therapeutic for the treatment, prevention, or reduction of cancer metastasis, angiogenesis (both cancer related and unrelated), and fibrosis.

### Lipocalin 2

Lipocalins are extracellular carriers of lipophilic molecules such as retinoids, steroids, and fatty acid, all of which may play important roles in the regulation of epithelial cell growth. We have discovered that lipocalin 2 polypeptides, or biologically active fragments or derivatives thereof, can reverse the EMT transition and can prevent or reduce conditions associated with EMT transitions including metastasis, angiogenesis, and fibrosis. Accordingly, the methods of the invention feature the use of lipocalin 2 for the prevention or reduction of metastatic, angiogenic, or fibrotic disorders in a mammal suffering from such a disorder.

Compounds useful in the methods of the invention include any lipocalin 2 polypeptide, analog, homolog, fragment or derivative thereof, or a nucleic acid sequence encoding a lipocalin 2 polypeptide, analog, homolog, fragment, or derivative thereof, wherein the polypeptide has an amino acid sequence that is substantially identical to at least a portion of lipocalin 2 (SEQ ID NOs: 2 and 4, for amino acid sequences and SEQ ID NOs: 1 and 3 for nucleic acid sequences) and has lipocalin 2 biological activity (see below). Modifications to the primary structure itself by deletion, addition, or alteration of the amino acids incorporated into the lipocalin sequence during translation can be made without destroying the activity of the protein. Such modifications can be made to improve expression, stability, solubility, cellular uptake, or biological activity of the protein in the various expression systems. For example, a mutation can increase the iron loading and intracellular iron unloading kinetics of a lipocalin-siderophore-iron complex. Generally, substitutions are made conservatively and take into consideration the effect on biological activity. Mutations, deletions, or additions in nucleotide sequences constructed for expression of analog proteins or fragments thereof must, of course, preserve the reading frame of the coding sequences and preferably will not create complementary regions that could hybridize to produce secondary mRNA structures such as loops or hairpins which would adversely affect translation of the mRNA.

Additional useful lipocalin 2 compounds include any peptidyl or non-peptidyl compound that is a lipocalin 2 analog and has or induces lipocalin 2 biological activity; any peptidyl or non-peptidyl compound that binds to a lipocalin 2 receptor (e.g., 24p3R in mouse cells; see Devireddy et al., Cell 123:1293-1305, 2005); any peptidyl or non-peptidyl lipocalin 2 receptor agonists, including but not limited to agonistic antibodies; any compound known to stimulate or increase blood serum levels of lipocalin 2 polypeptides or increase the biological activity of lipocalin 2 polypeptides; any compound known to decrease the expression or biological activity of a lipocalin 2 inhibitor (e.g., an inhibitor that blocks binding to a siderophore or a lipocalin 2 receptor); and any compound that mimics lipocalin 2 effects on reducing raf, MEK, or ERK1/2 phosphorylation and/or biological activity.

Lipocalin 2 biological activity includes binding to an iron siderophore, binding to or transporting iron, binding to small molecular weight ligands, binding to the lipocalin 2 receptor (see Devireddy et al., supra), retinol transport, cryptic coloration, olfaction, pheromone transport, and prostaglandin synthesis, apoptosis (see Akerstrom et al., Biochim. Biophy. Acta 1482:1-8, 2000; and Flower et al., Biochem. J. 318:1-14, 1996. Assays for lipocalin 2 biological activity include assays for siderophore binding, iron binding, iron uptake (e.g., analysis of expression of ferritin protein levels and calorimetric determination of intracellular iron), and receptor binding as described in Hanai et al., supra, Mori et al., supra, Li et al., supra, Yang et al., supra, and Devireddy et al., supra), and apoptotic assays known in the art.

Lipocalin 2 polypeptides can be produced by any of a variety of methods for protein production known in the art such as purification of naturally occurring lipocalin 2 products, products of chemical synthetic procedures, and products produced by recombinant techniques from a prokaryotic or eukaryotic host, including, for example, bacterial, fungus, higher plant, insect and mammalian cells. In one example, lipocalin 2 is produced by recombinant DNA methods by inserting a DNA sequence encoding lipocalin 2, or fragments or derivatives thereof, into a recombinant expression vector and expressing the DNA sequence under conditions promoting expression. General techniques for nucleic acid manipulation are described, for example, by Sambrook et al., in “Molecular Cloning: A Laboratory Manual,” 2nd Edition, Cold Spring Harbor Laboratory press, 1989; Goeddel et al., in “Gene Expression Technology: Methods in Enzymology,” Academic Press, San Diego, Calif., 1990; Ausubel et al., in “Current Protocols in Molecular Biology,” John Wiley & Sons, New York, N.Y., 1998; Watson et al., “Recombinant DNA,” Chapter 12, 2nd edition, Scientific American Books, 1992; and other laboratory textbooks. The DNA encoding lipocalin 2 is operably linked to suitable transcriptional or translational regulatory elements derived from mammalian, viral, or insect genes. Such regulatory elements include a transcriptional promoter, an optional operator sequence to control transcription, a sequence encoding suitable mRNA ribosomal binding sites, and sequences which control the termination of transcription and translation. The ability to replicate in a host, usually conferred by an origin of replication, and a selection gene to facilitate recognition of transformants may additionally be incorporated.

Appropriate cloning and expression vectors for use with bacterial, fungal, yeast, and mammalian cellular hosts can be found, for example, in “Cloning Vectors: A Laboratory Manual,” Elsevier, New York, 1985, the relevant disclosure of which is hereby incorporated by reference.

The expression construct is introduced into the host cell using a method appropriate to the host cell, as will be apparent to one of skill in the art. The expression construct can be introduced for transient expression of the protein or stable expression by selecting cells using a selectable marker in order to generate a stable cell line that expresses the protein continuously. A variety of methods for introducing nucleic acids into host cells are known in the art, including, but not limited to, electroporation; transfection employing calcium chloride, rubidium chloride, calcium phosphate, DEAE-dextran, or other substances; microprojectile bombardment; lipofection; and infection (where the vector is an infectious agent).

Suitable host cells for expression of lipocalin 2 from recombinant vectors include prokaryotes, fungal, mammalian cells, or insect cells.

Purified lipocalin 2, or fragments or derivatives thereof, are prepared by culturing suitable host/vector systems to express the recombinant proteins. As a secreted protein, lipocalin 2 is likely to be released from the membrane and can then be purified from culture media or cell extracts.

In one example, supernatants from systems which secrete recombinant protein into culture media can be first concentrated using a commercially available protein concentration filter, for example, an Amicon or Millipore Pellicon ultrafiltration unit, and the purified.

In addition to the methods employing recombinant DNA, lipocalin 2 polypeptides, or fragments of analogs thereof, can be purified from sources that naturally produce the soluble form of the protein. Examples of these sources include any mammalian tissue or cells, such as stomach, pancreas, colon, larynx, ischemic kidney, and neutrophils, and SV40 transformed cell lines. The lipocalin 2 from these sources can be purified and concentrated using any of the methods known in the art or described above.

After purification, lipocalin 2 may be exchanged into different buffers and/or concentrated by any of a variety of methods known to the art, including, but not limited to, filtration and dialysis. The purified lipocalin 2 is preferably at least 85% pure, more preferably at least 95% pure, and most preferably at least 98% pure. Regardless of the exact numerical value of the purity, the lipocalin 2 is sufficiently pure for use as a pharmaceutical product.

Lipocalin 2 polypeptides, or fragments or analogs thereof, can also be produced by chemical synthesis (e.g., by the methods described in “Solid Phase Peptide Synthesis,” 2^{nd} ed., The Pierce Chemical Co., Rockford, Ill., 1984). Modifications to the protein, such as those described below, can also be produced by chemical synthesis.

Lipocalin 2 Modifications

The invention encompasses lipocalin 2 polypeptides, or fragments or derivatives thereof, which are modified during or after synthesis or translation. Modifications may provide additional advantages such as increased affinity, decreased off-rate, solubility, stability and in vivo or in vitro circulating time of the polypeptide, or decreased immunogenicity and include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Creighton, “Proteins: Structures and Molecular Properties,” 2d Ed., W. H. Freeman and Co., N.Y., 1992; “Postranslational Covalent Modification of Proteins,” Johnson, ed., Academic Press, New York, 1983; Seifter et al., Meth. Enzymol., 182:626-646, 1990; Rattan et al., Ann. NY Acad. Sci., 663:48-62, 1992). Additionally, the lipocalin 2 polypeptide may contain one or more non-classical amino acids. Non-classical amino acids include, but are not limited to, to the D-isomers of the common amino acids, 2,4-diaminobutyric acid, α-amino isobutyric acid, 4-aminobutyric acid, Abu, 2-amino butyric acid, g-Abu, e-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, Ca-methyl amino acids, Na-methyl amino acids, and amino acid analogs in general. Furthermore, the amino acid can be D (dextrorotary) or L (levorotary). For example, lipocalin 2 has an unpaired cysteine which can be used for coupling to larger polymers.

Additional post-translational modifications encompassed by the invention include, for example, e.g., N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of prokaryotic host cell expression.

As described above, the invention also includes chemically modified derivatives of lipocalin 2, which may provide additional advantages such as increased solubility, stability and circulating time of the polypeptide, or decreased immunogenicity (see U.S. Pat. No. 4,179,337). The chemical moieties for derivitization may be selected from water soluble polymers such as, for example, polyethylene glycol, ethylene glycol/propylene glycol copolymers, carboxymethylcellulose, dextran, polyvinyl alcohol and the like. The lipocalin 2 polypeptide may be modified at random positions within the molecule, or at predetermined positions within the molecule and may include one, two, three or more attached chemical moieties.

The polymer may be of any molecular weight, and may be branched or unbranched. For polyethylene glycol, the preferred molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog). As noted above, the polyethylene glycol may have a branched structure. Branched polyethylene glycols are described, for example, in U.S. Pat. No. 5,643,575; Morpurgo et al., Appl. Biochem. Biotechnol. 56:59-72, (1996); Vorobjev et al., Nucleosides Nucleotides 18:2745-2750, (1999); and Caliceti et al., Bioconjug. Chem. 10:638-646, (1999), the disclosures of each of which are incorporated by reference.

The polyethylene glycol molecules (or other chemical moieties) should be attached to the lipocalin 2 polypeptide with consideration of effects on functional or antigenic domains of the protein. There are a number of attachment methods available to those skilled in the art, e.g., EP 0 401 384, herein incorporated by reference (coupling PEG to G-CSF), see also Malik et al., Exp. Hematol. 20:1028-1035, (1992) (reporting pegylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues glutamic acid residues and the C-terminal amino acid residue. Sulfhydryl groups may also be used as a reactive group for attaching the polyethylene glycol molecules. Preferred for therapeutic purposes is attachment at an amino group, such as attachment at the N-terminus or lysine group. The number of polyethylene glycol moieties attached to each polypeptide of the invention (i.e., the degree of substitution) may also vary. For example, the pegylated lipocalin 2 may be linked, on average, to 1, 2, 3, 4, 5, 6, 7, 8, 9, 10, 12, 15, 17, 20, or more polyethylene glycol molecules. Similarly, the average degree of substitution may range within ranges such as 1-3, 2-4, 3-5, 4-6, 5-7, 6-8, 7-9, 8-10, 9-11, 10-12, 11-13, 12-14, 13-15, 14-16, 15-17, 16-18, 17-19, or 18-20 polyethylene glycol moieties per polypeptide molecule. Methods for determining the degree of substitution are discussed, for example, in Delgado et al., Crit. Rev. Thera. Drug Carrier Sys., 9:249-304, 1992.

The lipocalin 2 polypeptides may also be modified with a detectable label, including, but not limited to, an enzyme, prosthetic group, fluorescent material, luminescent material, bioluminescent material, radioactive material, positron emitting metal, nonradioactive paramagnetic metal ion, and affinity label for detection and isolation of a lipocalin 2 target. The detectable substance may be coupled or conjugated either directly to the polypeptides of the invention or indirectly, through an intermediate (such as, for example, a linker known in the art) using techniques known in the art. Examples of suitable enzymes include horseradish peroxidase, alkaline phosphatase, beta-galactosidase, glucose oxidase or acetylcholinesterase; examples of suitable prosthetic group complexes include streptavidin/biotin and avidin/biotin; examples of suitable fluorescent materials include biotin, umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include a radioactive metal ion, e.g., alpha-emitters or other radioisotopes such as, for example, iodine (¹³¹I, ¹²⁵I, ¹²³I, ¹²¹I), carbon (¹⁴C), sulfur (³⁵S), tritium (³H), indium (¹¹⁵mIn, ¹¹³mIn, ¹¹²In, ¹¹¹In), and technetium (⁹⁹Tc, ⁹⁹mTc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, Lu, ¹⁵⁹Gd, ¹⁴⁹Pm, ¹⁴⁰La, ¹⁷⁵Yb, ¹⁶⁶Ho, ⁹⁰Y, ⁴⁷Sc, ⁸⁶R ¹⁸⁸Re, ¹⁴²Pr; ¹⁰⁵Rh, ⁹⁷Ru, ⁶⁸Ge, ⁵⁷Co, ⁶⁵Zn, ⁸⁵Sr, ³²P, ¹⁵³Gd, 169Yb, ⁵¹Cr, ⁵⁴Mn, ⁷⁵Se, ¹¹³Sn, and ¹¹⁷Tin. The detectable substance may be coupled or conjugated either directly to the lipocalin 2 polypeptide or indirectly, through an intermediate (such as, for example, a linker known in the art) using techniques known in the art. See, for example, U.S. Pat. No. 4,741,900 for metal ions, which can be conjugated to lipocalin 2 polypeptide for use as diagnostics according to the present invention.

The lipocalin 2 polypeptide can also be modified by conjugation to another protein or therapeutic compound. Such conjugation can be used, for example, to enhance the stability or solubility of the protein, to reduce the antigenicity, or to enhance the therapeutic effects of the protein. A preferred fusion protein comprises a heterologous region from immunoglobulin (e.g., all or part of the Fc region) that is useful to solubilize proteins (EP-A 0232 262).

A lipocalin 2 polypeptide of the invention may be conjugated to a therapeutic moiety such as a cytotoxin, e.g., a cytostatic or cytocidal agent, a chemotherapeutic agent, a radiotherapeutic agent or a radioactive metal ion, e.g., alpha-emitters such as, for example, ²¹³Bi, or other radioisotopes such as, for example, ¹⁰³Pd, ¹³³Xe, ¹³¹I, ⁶⁸Ge, ⁵⁷Co, ⁶⁵Zn, ⁸⁵Sr, ³²P, ³⁵S, ⁹⁰Y, ¹⁵³Sm, ¹⁵³Gd, ¹⁶⁹Yb, ⁵¹Cr, ⁵⁴Mn, ⁷⁵Se, ¹¹³Sn, ⁹⁰Yttrium, ¹¹⁷Tin, ¹⁸⁶Rhenium, ¹⁶⁶Holmium, and ¹⁸⁸Rhenium.

A cytotoxin or cytotoxic agent includes any agent that is detrimental to cells. Examples include, but are not limited to, paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, thymidine kinase, endonuclease, RNAse, and puromycin and fragments, variants or homologs thereof.

Additional therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclophosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Techniques known in the art may be applied to label lipocalin 2 polypeptides of the invention. Such techniques include, but are not limited to, the use of bifunctional conjugating agents (see, e.g., U.S. Pat. Nos. 5,756,065; 5,714,631; 5,696,239; 5,652,361; 5,505,931; 5,489,425; 5,435,990; 5,428,139; 5,342,604; 5,274,119; 4,994,560; and 5,808,003; the relevant disclosures of each of which are hereby incorporated by reference in its entirety) and direct coupling reactions (e.g., Bolton-Hunter and Chloramine-T reaction).

### The Role of Iron and Iron-Siderophore Complexes

Lipocalin 2 is member of a super family of carrier proteins that can complex and transport iron, typically via a siderophore. We have discovered that the effect of lipocalin 2 on EMT and on processes associated with EMT, such as metastasis, fibrosis, and angiogenesis, is enhanced when the protein is in a complex with a siderophore or iron-siderophore. Therefore, the invention also features the use of iron, siderophores, or both in addition to lipocalin-2. The invention also features lipocalin 2-siderophore or lipocalin 2-siderophore-iron complexes in the methods of the invention.

Under physiological conditions, most commonly occurring ionic forms of iron are very weakly soluble in water and, consequently, there is a very low concentration of free iron (III) ions in nature. In order to scavenge low amounts of iron from the medium, many microbes, including pathogenic bacteria such as Pseudomonas aeruginosa, Escherichia coli, and Salmonella typhimurium and fungi, produce and utilize very specific low molecular weight iron chelators known as siderophores. Siderophores are small protein molecules that scavenge iron from the environment and have a low molecular weight ranging from about 500 to about 1000 MW. Siderophores can be synthetic or naturally-occurring products harvested from bacterial cultures, and are commercially available. Siderophores are avidly taken up by lipocalin 2 when mixed together under physiological conditions in a wide variety of commonly used buffers including 10 mM Tris or phosphate-buffered saline. Typically, siderophores can be added in excess to a known quantity of lipocalin 2 protein. Lipocalin 2 molecules will bind to siderophore molecules such that each complex will contain one molecule of each species. Exogenous siderophores contemplated for use in the invention include, but are not limited to bacterial catecholate-type ferric siderophores (see Goetz et al., supra) enterochelin, carboxymycobactin, aminochelin, desfenioxamine, aerobactin, arthrobactin, schizokinen, foroxymithine, pseudobactins, neoenactin, photobactin, fenichrome, hemin, achromobactin, achromobactin, rhizobactin, and other bacterial products, as well as citrate and synthetic analogs and moieties and others that can be produced using organic chemistry processes.

The present invention includes the use of iron replacements, which can be administered either orally or intravenously, as is used for the treatment of CKD or anemia. Iron replacements are known to the skilled artisan and include ferrous sulfate, ferrous femarate, and ferrous gluconate. For intravenous use, dextran-iron is preferred. Dosages can be determined by the physician but, in generally, the dosages for oral iron replacements are such that the elemental iron is delivered at a concentration of about 60-180 mg per day for oral administration and about 100 mg per day or elemental iron for IV administration, as needed. The iron can be administered separately or as a previously formed complex with lipocalin 2. The invention also includes the use of siderophores, which can be administered separately or as a previously formed complex with lipocalin 2 and/or iron.

### Therapeutic Uses of the Invention

We have discovered that lipocalin 2 reverses the EMT process that is associated with a variety of cellular processes including cancer metastasis, angiogenesis, and fibrosis. We have also discovered that lipocalin 2 reduces VEGF production and induces thrombospondin expression, both of which contribute to the anti-angiogenic and anti-metastatic effects of lipocalin 2. The therapeutic application for the use of lipocalin 2, or biologically active fragments or derivates thereof for the treatment or prevention of cancer, cancer metastasis, angiogenesis, and fibrosis are described below. Our discovery is further supported by the recent publication by Lee et al., (Int. J. Cancer, online publication Dec. 27, 2005, hereby incorporated by reference in its entirety) demonstrating that expression of lipocalin 2 (NGAL) is highly expressed in colon cancer cell lines that were poorly metastatic. Furthermore, the authors demonstrated that ectopic expression of lipocalin 2 suppressed the invasiveness of colon cancer cells in an in vitro model and inhibited liver metastasis in an experimental animal model. These results are in agreement with our discovery that lipocalin 2 can be used to treat, prevent, or reduce cancer metastasis.

The various disorders that can be treated or prevented using the methods of the invention are described below. It should be noted that each of the disorders described can be considered a separate disorder or can be a part of an additional disorder, for example, angiogenic disorders can be included as a component of metastasis but can also be included as a separate group of disorders not related to cancer.

### Cancer Applications

We have discovered that lipocalin 2 reverses the EMT process generally and specifically, that lipocalin 2 reversed ras induced EMT. We have discovered that lipocalin 2 converts 4T1-ras transformed mesenchymal tumor cells to an epithelial phenotype, increases E-cadherin expression, and suppresses cell invasiveness in vitro. We have shown that lipocalin 2 provides a protective role during ras mediated transformation and metastasis in vitro and in vivo. Indeed, the lipocalin 2 treated cells produced smaller, more coherent tumors of higher density (similar weight but different cell types), with less regional invasion and dramatically fewer metastases in vivo, (as assessed by lung weight, by the number of nodules on the lung surface, and histology). Accordingly, the invention includes the use of lipocalin 2, or fragments or derivatives thereof, to treat, prevent, or reduce cancer and specifically cancer metastasis. Of particular importance to the present invention are subjects (e.g., humans and other mammals) diagnosed with and/or treated for a primary tumor, including prophylactic treatment of at-risk subjects, but not yet diagnosed with metastatic disease or determined to lack metastatic disease, and those subjects otherwise predisposed to developing metastatic disease. The methods of the invention can be used to prevent the occurrence or re-occurrence of metastatic disease. Also included are subjects who have undergone treatment for metastasis or a possible metastasis in order to prevent or reduce metastatic disease. The methods of the invention can be used before during or after additional therapies to treat the primary tumor, the metastases, or the risk of either.

The term cancer embraces a collection of malignancies with each cancer of each organ consisting of numerous subsets. Typically, at the time of cancer diagnosis, “the cancer” consists in fact of multiple subpopulations of cells with diverse genetic, biochemical, immunologic, and biologic characteristics. Benign or malignant growths of cancer are referred to as tumors. The tumor can be a solid tumor or a non-solid or soft tissue tumor. Examples of soft tissue tumors include leukemia (e.g., chronic myelogenous leukemia, acute myelogenous leukemia, adult acute lymphoblastic leukemia, acute myelogenous leukemia, mature B-cell acute lymphoblastic leukemia, chronic lymphocytic leukemia, polymphocytic leukemia, or hairy cell leukemia), or lymphoma (e.g., non-Hodgkin's lymphoma, cutaneous T-cell lymphoma, or Hodgkin's disease). Solid tumors can be further separated into those of epithelial cell origin and those of non-epithelial cell origin. Examples of epithelial cell solid tumors include tumors of the gastrointestinal tract, colon, breast, prostate, lung, kidney, liver, pancreas, ovary, head and neck, oral cavity, stomach, duodenum, small intestine, large intestine, anus, gall bladder, labium, nasopharynx, skin, uterus, male genital organ, urinary organs, bladder, and skin. Solid tumors of non-epithelial origin include sarcomas, brain tumors, and bone tumors. While the methods of the invention can be used to treat any tumor or tumor metastasis, lipocalin 2 is preferably used for the treatment or prevention of epithelial cell solid tumor metastasis.

Almost any cancer can metastasize. The metastases may occur to any site, however some cancers preferentially metastasize to particular organs. For example, lung cancer metastasizes to brain, bone, liver, adrenal glands, lung, pleura, subcutaneous tissue, kidney, lymph nodes, cerebrospinal fluid, pancreas, or bone marrow. Breast cancer typically metastasizes to lymph nodes, breast, abdominal viscera, lungs, bones, liver, adrenal glands, brain, meninges, pleura, or the cerebrospinal fluid. Head and neck cancer typically metastasizes to lung, esophagus, upper digestive tract, lymph nodes, oral cavity, or the nasal cavity. Cervical cancer typically metastasizes to vagina, paracervical spaces, bladder, rectum, pelvic wall, or the lymph nodes. Bladder cancer typically metastasizes to prostate, uterus, vagina, bowel, pelvic wall, lymph nodes, and or perivesical fat. Metastases, particularly micormetastases, or metastases that are too small to be seen, can be difficult to diagnose. If there are individual cells, or even small areas of growing cells elsewhere in the body, there is no scan that is detailed enough to spot them. For a few tumours, there are blood tests that detect proteins released by the cancer cells (e.g., CA-125 for ovarian cancer, PSA for prostate cancer). But for most cancers, there is no blood test that can say whether a cancer has spread or not and diagnosis of metastatic disease only occurs after the cancer has spread extensively. As a result, most cancer-related deaths result from metastases that are difficult to detect or to completely eradicate by surgery, radiation, drugs, and/or biotherapy.

Once a primary tumor is diagnosed in a patient, it is possible that the primary tumor will progress and spread to the regional lymph nodes and to distant organs. This process is defined as metastasis. Primary tumors are classified by the type of tissue from which they arise, metastatic tumors are classified by the tissue type from which the cancer cells are derived. For solid tumors to invade and metastasize, the epithelial cell changes its phenotype, from one that is polarized and that grows appositionally to one that is mobile, more-fibroblast like, and invasive. This so-called epithelial to mesenchymal transition (EMT) is a rather general phenomenon that correlates with tumor progression. Generally, EMT is believed to occur because of the activation of a dominantly acting oncogene or as a result of a loss of tumor suppressor gene activity. Given our findings that lipocalin 2 acts as an epithelial inducer and a suppressor of metastasis by reversing EMT, possibly through restoration of E-cadherin expression via effects on the ras-MAPK signaling pathway, the methods of the invention are preferably used for the treatment or prevention of metastatic disease.

Non-limiting examples of metastatic disease and the conventional methods used to treat the metastases are described below.

Brain Metastases

Brain metastases develop when tumor cells that originate in tissues outside the central nervous system (CNS) spread secondarily to directly involve the brain. Intracranial metastases may involve the brain parenchyma, the cranial nerves, the blood vessels (including the dural sinuses), the dura, the leptomeninges, and the inner table of the skull. Of the intracranial metastases, the most common are intraparenchymal metastases. The frequency of brain metastasis by primary tumor is lung (48%), breast (15%), melanoma (9%), colon (5%), other known primary (13%), and other unknown primary tumors (11%). See Loeffler et al., “Metastatic Brain Cancer,” in “Cancer: Principles & Practice Of Oncology,” pp. 2523-2536, DeVita et al., editors, 5th ed., 1997. Symptoms associated with brain metastasis include altered mental status, hemiparesis, hemisensory loss, papilledema, and gait ataxia. Thus, patients newly diagnosed with brain metastases are often placed on anticonvulsant prophylaxis and corticoseteroids for prolonged periods of time. Such drugs include phenyloin sodium and phenobarbital.

Brain metastases can be treated surgically with excision of the metastases if they are easily reached. With the advancement in imaging and localization techniques, the morbidity associated with surgical removal of brain metastases has decreased. However, risks still remain. Radiotherapy is therefore a mainstay of the treatment of patients with brain metastases. Radiotherapy may be combined with surgery as an adjuvant treatment to surgery. Alternatively, radiosurgery may be used. Radiosurgery is a technique of external irradiation that uses multiple convergent beams to deliver a high single dose of radiation to a small volume. Thus, in one embodiment, the invention includes the use of lipocalin 2 in combination with radiotherapy or radiosurgery.

Lung Metastases

The lungs are the second most frequent site of metastatic disease. Anatomically, the lungs are vascular rich sites and the first capillary bed encountered by circulating tumor cells as they exit from the venous drainage system of their primary tumor. Thus, the lungs act as the initial filtration site, where disseminated tumor cells become mechanically trapped. However, the cells which get trapped there and go on to proliferate and form metastatic lesions will largely depend upon the original primary tumor from which they derive. This hematogenous process of lung metastases is the most common means, but pulmonary metastases can also occur via the lymphatic system. See Pass et al., “Metastatic Cancer to the Lung,” in “Cancer: Principles & Practice of Oncology,” pp. 2536-2551, DeVita et al., editors, 5th ed., 1997.

The most common primary tumors which go on to have lung metastases include soft tissue sarcoma, colorectal carcinoma, germ cell tumors, osteosarcoma, certain pediatric tumors (e.g., rhabdomyosarcomas, Ewing's sarcomas, Wilm's tumor, liposarcomas, leiomyosarcomas, alveolar sarcomas, synovial sarcomas, fibrosarcomas, neurogenic sarcomas, and epithelial sarcomas), melanoma, renal cell carcinoma, and breast carcinoma. Most of the metastases from these primary tumors are treated surgically. However, some recommend surgery in combination with chemotherapy. For example, germ cell tumors which have metastasized to the lung are treated with surgical resection following curative cisplatin-based combination chemotherapy.

Treatment of lung metastases frequently involves metastasectomy, i.e., surgical removal of the lung metastatic lesion. Thus, one aspect of the invention includes the use of lipocalin 2 in combination with conventional therapies, as discussed herein or as known in the art, for the treatment of lung metastases.

Liver Metastases

Metastatic disease in the liver can occur from many primary tumor sites. Because of anatomic venous drainage, gastrointestinal tumors spread preferentially to the liver, such that many patients are initially diagnosed with cancer in the liver. With most gastrointestinal tumors that metastasize to the liver, the diagnosis is dire with relatively short survival. But, colorectal metastases to the liver may be amenable to treatment after resectional therapy.

Systemic chemotherapy represents the modality most frequently used in the treatment of hepatic metastases. Response to systemic chemotherapy varies depending on the primary tumor. Another therapy option is hepatic arterial chemotherapy. Liver metastases are perfused almost exclusively by the hepatic artery, while normal hepatocytes derive their blood from both the portal vein and the hepatic artery. Thus, hepatic arterial chemotherapy, wherein 3H-floxuridine (3H-FUDR) (or other chemotherapeutic agent or agents) is injected into the hepatic artery, results in significantly increased drug concentrations (15 fold) in the metastases than in normal liver tissue. Additional drugs administered via the hepatic artery include but are not limited to fluorouracil, 5-fluorouracil-2-deoxyuridine, bischlorethylnitrosourea, mitomycin C, cisplatin, and doxorubicin.

For a metastasis to the liver, treatment modalities can include systemic chemotherapy (using for example 3H-floxuridine), intrahepatic therapy, hepatic artery ligation or embolization, chemoembolization, radiation therapy, alcohol injection, and cryosurgery. For chemoembolization, the following drug regimens can be used (1) DSM and mitomycin C; (2) collagen, cisplatin, doxorubicin, and mitomycin C; (3) fluorouracil, mitomycin C, ethiodized oil, and gelatin; (4) angiostatin (or other drug which inhibits neovascularization or angiogenesis), cisplatin, doxorubicin, and mitomycin C; (5) lipiodol and doxorubicin; (6) gel foam, doxorubicin, mitomycin C, and cisplatin; (7) doxorubicin, mitomycin C, and lipiodol; and (8) polyvinyl, alcohol, fluorouracil, and interferon. For additional treatments and detail, see Daly et al., “Metastatic Cancer to the Liver,” in “Cancer: Principles & Practice of Oncology,” pp. 2551-2569, DeVita et al., editors, 5th ed., 1997.

Thus, one aspect of the invention includes the use of lipocalin 2 in combination with any of the available treatment therapies, as discussed herein or as known in the art, for the treatment of liver metastases.

Bone Metastases

Treatment of bone metastases is best approached using a multimodality methodology. One of the problems with bone is the incidence of bone fracture and bone healing. Tumor mass for bone tumors can be performed surgically and can include amputation of a limb. In addition to surgical treatment, radiation can be used on skeletal metastases. Localized external radiation, hemibody radiation, or systemic radionuclide therapy can be considered for widely disseminated bone disease. Bone seeking isotopes such as ⁸⁹Sr are advocated as they are better tolerated than ³²P-orthophosphate, which is a high-energy isotope. For additional modalities and details for treating bone metastases, see, e.g., Healy, “Metastatic Cancer to the Bone,” in “Cancer: Principles & Practice of Oncology,” pp. 2570-2586, DeVita et al., editors, 5th ed., 1997.

Thus, one aspect of the invention includes the use of lipocalin 2 in combination with any of the available treatment therapies, as discussed herein or as known in the art, for the treatment of bone metastases.

### Combination Therapies for Cancer and Metastatic Disease

In various embodiments lipocalin 2 nucleic acids or polypeptides can be provided in conjunction (e.g., before, during, or after) with additional cancer therapies to prevent or reduce tumor growth or metastasis. Treatment therapies include but are not limited to surgery, radiation therapy, chemotherapy, biologic therapy (e.g., cytokines, immunotherapy, and interferons), differentiating therapy, immune therapy, anti-angiogenic therapy, hormone therapies, or hyperthermia. Lipocalin 2 compounds may be formulated alone or in combination with any additional cancer therapies in a variety of ways that are known in the art. Such additional cancer therapies can be administered before, during, or after the administration lipocalin 2 nucleic acids or polypeptides, or fragments or derivatives thereof.

Chemotherapeutic agents include, without limitation, asparaginase, bleomycin, busulfan carmustine (commonly referred to as BCNU), chlorambucil, cladribine (commonly referred to as 2-CdA), CPT11, cyclophosphamide, cytarabine (commonly referred to as Ara-C), dacarbazine, daunorubicin, dexamethasone, doxorubicin (commonly referred to as Adriamycin), etoposide, fludarabine, 5-fluorouracil (commonly referred to as 5FU), hydroxyurea, idarubicin, ifosfamide, interferon-α (native or recombinant), levamisole, lomustine (commonly referred to as CCNU), mechlorethamine (commonly referred to as nitrogen mustard), melphalan, mercaptopurine, methotrexate, mitomycin, mitoxantrone, paclitaxel, pentostatin, prednisone, procarbazine, tamoxifen, taxol-related compounds, 6-thiogaunine, topotecan, vinblastine, and vincristine. The dosage of the chemotherapeutic agent will be determined by the physician and will depend on other clinical factors such as weight and condition of the human or animal and the route of administration of the compound.

In addition, the invention provides for the use of an angiogenesis inhibitor used in combination with any of the lipocalin 2 compounds to treat cancer or cancer metastasis. Angiogenesis inhibitors, also known as anti-angiogenic agents, that may be used in combination with any of the lipocalin 2 compounds include an antibody, an antibody that binds VEGF-A, an antibody that binds a VEGF receptor and blocks VEGF binding, avastin, endostatin, angiostatin, restin, tumstatin, TNP-470, 2-methoxyestradiol, thalidomide, a peptide fragment of an anti-angiogenic protein, canstatin, arrestin, a VEGF kinase inhibitor, CPTK787, SFH-1, an anti-angiogenic protein, thrombospondin-1, platelet factor-4, interferon-α, an agent that blocks TIE-1 or TIE-2 signaling, or PIH12 signaling, an agent that blocks an extracellular vascular endothelial (VE) cadherin domain, an antibody that binds to an extracellular VE-cadherin domain, tetracycline, penicillamine, vinblastine, cytoxan, edelfosine, tegafur or uracil, curcumin, green tea, genistein, resveratrol, N-acetyl cysteine, captopril, a cox-2 inhibitor, celecoxib, and rofecoxib.

The dosage of the angiogenesis inhibitor will depend on other clinical factors such as weight and condition of the human or animal and the route of administration of the compound. For treating humans or animals, between approximately 0.5 mg/kg to 500 mg/kg body weight of the angiogenesis inhibitor can be administered. A more preferable range is 1 mg/kg to 100 mg/kg body weight with the most preferable range being from 2 mg/kg to 50 mg/kg body weight. Depending upon the half-life of the angiogenesis inhibitor in the particular animal or human, the angiogenesis inhibitor can be administered between several times per day to once a week. The methods of the present invention provide for single as well as multiple administrations, given either simultaneously or over an extended period of time.

In addition, the invention provides for the use of an anti-proliferative compound used in combination with any of the lipocalin 2 compounds for treating a tumor. Anti-proliferative compounds that may be used in combination with any of the lipocalin 2 compounds include taxol, troglitazone, an antibody that binds bFGF, an antibody that binds bFGF-saporin, a statin, an ACE inhibitor, suramin, 17 beta-estradiol, atorvastatin, fluvastatin, lovastatin, pravastatin, simvastatin, cerivastatin, perindopril, quinapril, captopril, lisinopril, enalapril, fosinopril, cilazapril, ramipril, and a kinase inhibitor.

The dosage of the anti-proliferative compound depends on clinical factors such as weight and condition of the human or animal and the route of delivery of the compound. In general, for treating humans or animals, between approximately 0.1 mg/kg to 500 mg/kg body weight of the anti-proliferative compound can be administered. A more preferable range is 1 mg/kg to 50 mg/kg body weight with the most preferable range being from 1 mg/kg to 25 mg/kg body weight. Depending upon the half-life of the anti-proliferative compound in the particular animal or human, the compound can be administered between several times per day to once a week. The methods of the present invention provide for single as well as multiple administrations, given either simultaneously or over an extended period of time.

It should be noted that although each of the compounds is listed under a specific category of compounds, these categories are not meant to be limiting in scope. Many of the compounds possess more than one activity and can therefore be included under more than one category.

For each of the compounds listed, all of the modes of administration described above can be used. As some of the compounds described have shown toxicity when administered orally or systemically, local administration can also be used. In general, percent composition of the compound will range from 0.05% to 50% weight for weight of compound to coating material used.

### Angiogenesis Applications

Angiogenesis is a complex, combinatorial process that is regulated by a balance between pro- and anti-angiogenic molecules. Angiogenic stimuli (e.g. hypoxia or inflammatory cytokines) result in the induced expression and release of angiogenic growth factors such as vascular endothelial growth factor (VEGF) or fibroblast growth factor (FGF). These growth factors stimulate endothelial cells (EC) in the existing vasculature to proliferate and migrate through the tissue to form new endothelialized channels. There are a variety of diseases in which angiogenesis is believed to be important, referred to as angiogenic diseases or disorders, including but not limited to, as inflammatory disorders such as immune and non-immune inflammation, rheumatoid arthritis, ocular neovascular disease, choroidal retinal neovascularization, osteoarthritis, chronic articular rheumatism, psoriasis, disorders associated with inappropriate or inopportune invasion of vessels such as diabetic retinopathy, neovascular glaucoma, restenosis, capillary proliferation in atherosclerotic plaques and osteoporosis, cancer associated disorders, such as solid tumors, solid tumor metastases, hematopoetic tumors or metastases, angiofibromas, retrolental fibroplasia, hemangiomas, Kaposi's sarcoma, and cancers or cancer metastases, which require neovascularization to support tumor growth.

We have found that lipocalin 2 can cause cells to become less angiogenic. Transformation by the oncogene ras leads to both EMT and promotes angiogenesis and lipocalin can reverse both effects. Furthermore, we have discovered that lipocalin 2 blocks VEGF, an angiogenesis inducer, and induces thrombospondin, an inhibitor of angiogenesis. Thus, lipocalin 2 can also be used as a therapeutic to block blood vessel formation and to treat angiogenic diseases, including cancer metastasis associated that is characterized by angiogenesis.

Angiogenic disorders can be diagnosed using standard techniques known in the art, such as detection of markers of angiogenesis (e.g., increased VEGF and other pro-angiogenic molecules or decreased anti-angiogenic molecules). The therapeutic effectiveness of lipocalin 2, or fragments or derivatives thereof, can be measured using in vitro and in vivo assays well known in the art. For example Heeschen et al., J. Clin. Invest. 110:527-536, 2002. One particular assay measures angiogenesis in the chick chorioallantoic membrane (CAM) and is referred to as the CAM assay. The CAM assay has been described in detail by others, and further has been used to measure both angiogenesis of tumor tissues. See Ausprunk et al., Am. J. Pathol., 79:597-618, 1975; and Ossonski et al., Cancer Res., 40:2300-2309, 1980. The CAM assay is a well recognized assay model for in vivo angiogenesis because neovascularization of whole tissue is occurring, and actual chick embryo blood vessels are growing into the CAM or into the tissue grown on the CAM.

Another assay for measuring angiogenesis is the in vivo rabbit eye model and is referred to as the rabbit eye assay. The rabbit eye assay has been described in detail by others, and further has been used to measure both angiogenesis and neovascularization in the presence of angiogenic inhibitors such as thalidomide. See D'Amato et al., Proc. Natl. Acad. Sci. 91:4082-4085, 1994. The rabbit eye assay is a well recognized assay model for in vivo angiogenesis because the neovascularization process, exemplified by rabbit blood vessels growing from the rim of the cornea into the cornea, is easily visualized through the naturally transparent cornea of the eye. Additionally, both the extent and the amount of stimulation or inhibition of neovascularization or regression of neovascularization can easily be monitored over time.

A further assay for measuring angiogenesis in the chimeric mouse:human mouse model and is referred to as the chimeric mouse assay. The assay has been described in detail by others, and further has been described herein to measure angiogenesis, neovascularization, and regression of tumor tissues. See Yan, et al., J. Clin. Invest. 91:986-996, 1993. The chimeric mouse assay is a useful assay model for in vivo angiogenesis because the transplanted skin grafts closely resemble normal human skin histologically and neovascularization of whole tissue is occurring wherein actual human blood vessels are growing from the grafted human skin into the human tumor tissue on the surface of the grafted human skin. The origin of the neovascularization into the human graft can be demonstrated by immunohistochemical staining of the neovasculature with human-specific endothelial cell markers. The chimeric mouse assay demonstrates regression of neovascularization based on both the amount and extent of regression of new vessel growth. Furthermore, it is easy to monitor effects on the growth of any tissue transplanted upon the grafted skin, such as a tumor tissue.

### Fibrosis Applications

The EMT transition is a critical factor in the development of fibrotic conditions. We have discovered lipocalin 2 reverses or halts the EMT process that leads to fibrosis. Accordingly, lipocalin 2, or fragments or derivatives thereof, for the treatment or prevention of fibrotic disorders.

Collagen is a fibril-forming protein which is essential for maintaining the integrity of the extracellular matrix found in connective tissues. The production of collagen is a highly regulated process, and its disturbance may lead to the development of tissue fibrosis. While the formation of fibrous tissue is part of the normal beneficial process of healing after injury, in some circumstances there is an abnormal accumulation of fibrous materials such that it may ultimately lead to organ failure (Border et al., New Engl. J. Med. 331:1286-1292, 1994). Injury to any organ leads to a stereotypical physiological response: platelet-induced hemostasis, followed by an influx of inflammatory cells and activated fibroblasts. Cytokines derived from these cell types drive the formation of new extracellular matrix and blood vessels (granulation tissue). The generation of granulation tissue is a carefully orchestrated program in which the expression of protease inhibitors and extracellular matrix proteins is upregulated, and the expression of proteases is reduced, leading to the accumulation of extracellular matrix.

Central to the development of fibrotic conditions, whether induced or spontaneous, is stimulation of fibroblast activity. The influx of inflammatory cells and activated fibroblasts into the injured organ depends on the ability of these cell types to interact with the interstitial matrix comprised primarily of collagens.

Many of the diseases associated with the proliferation of fibrous tissue are both chronic and often debilitating, including for example, skin diseases such as scleroderma. Some, including pulmonary fibrosis, can be fatal due in part to the fact that the currently available treatments for this disease have significant side effects and are generally not efficacious in slowing or halting the progression of fibrosis (Nagler et al., Am. J. Respir. Crit. Care Med., 154:1082-1086, 1996).

A subject with a fibrotic condition refers to, but is not limited to, subjects afflicted with fibrosis of an internal organ, subjects afflicted with a dermal fibrosing disorder, and subjects afflicted with fibrotic conditions of the eye. Fibrosis of internal organs (e.g., liver, lung, kidney, heart blood vessels, and gastrointestinal tract), occurs in disorders such as pulmonary fibrosis, myelofibrosis, liver cirrhosis, mesangial proliferative glomerulonephritis, crescentic glomerulonephritis, diabetic nephropathy, renal interstitial fibrosis, renal fibrosis in patients receiving cyclosporin, and HIV associated nephropathy. Dermal fibrosing disorders include, but are not limited to, scleroderma, morphea, keloids, hypertrophic scars, familial cutaneous collagenoma, and connective tissue nevi of the collagen type. Fibrotic conditions of the eye include conditions such as diabetic retinopathy, postsurgical scarring (for example, after glaucoma filtering surgery and after cross-eye surgery), and proliferative vitreoretinopathy.

Additional fibrotic conditions which may be treated by the methods of the present invention include rheumatoid arthritis, diseases associated with prolonged joint pain and deteriorated joints, progressive systemic sclerosis, polymyositis, dermatomyositis, eosinophilic fascitis, morphea, Raynaud's syndrome, and nasal polyposis.

In addition, fibrotic conditions which may be treated by the methods of present invention also include overproduction of scarring in patients who are known to form keloids or hypertrophic scars, scarring or overproduction of scarring during healing of various types of wounds including surgical incisions, surgical abdominal wounds, and traumatic lacerations, scarring and reclosing of arteries following coronary angioplasty, excess scar or fibrous tissue formation associated with cardiac fibrosis after infarction and in hypersensitive vasculopathy.

Fibrotic conditions can be diagnosed using a variety of techniques known in the art including, for example, radiological methods to detect, for example, the diminution or atrophy of the overall size of the organ (e.g., the thinning of the cortex of the kidney on ultrasound or X ray), measurement of markers in the blood (e.g., blood urea nitrogen or creatinine for kidney fibrosis or bilirubin, SGPT, SGOT for liver fibrosis); biopsy and detection of scar tissue (e.g., glomerulosclerosis, scarring in the mesengium, or fibrous crescents in the glomerulus for kidney fibrosis); or detection of organ failure (e.g., portal hypertension leading to the development of ascites or upper gastrointestinal tract bleeding for liver fibrosis).

Lipocalin 2 can be provided locally or systemically for the prevention of fibrosis. In this context, lipocalin 2 and nucleic acids encoding the same may be administered for the treatment of chronic renal failure (fibrosis of the kidney), cirrhosis of the liver, scleroderma, bone marrow fibrosis, bone fibrosis, keloids, burn contractures, and surgical adhesions. For example, for the prevention of excessive surgical scarring lipocalin 2 may be provided locally on a biodegradable patch or from a drug-eluting object. Lipocalin 2 may also be used on or under the surfaces of medical devices (e.g., a stent) where fibrosis might otherwise occur.

Lipocalin 2 may be provided for the treatment of fibrotic conditions alone or in conjunction with other anti-fibrotic therapies or anti-fibrotic compounds. Anti-fibrotic compounds include an agent that blocks TGF-β signaling or inhibits activation of plasminogen activator inhibitor-1 promoter activity, an antibody that binds to TGF-β or to a TGF-β receptor, an antibody that binds to TGF-β receptor I, II, or III, a kinase inhibitor, an agent that blocks connective tissue growth factor (CTGF) signaling, an agent that inhibits prolyl hydroxylase, an agent that inhibits procollagen C-proteinase, pirfenidone, silymarin, pentoxifylline, colchicine, embrel, remicade, an agent that antagonizes TGF-β, an agent that antagonizes CTGF, and an agent that inhibits vascular endothelial growth factor VEGF.

The dosage of the anti-fibrotic agent will depend on other clinical factors such as weight and condition of the subject and the route of administration of the compound. For treating subjects, between approximately 0.1 mg/kg to 500 mg/kg body weight of the anti-fibrotic agent can be administered. A more preferable range is 1 mg/kg to 50 mg/kg body weight with the most preferable range being from 1 mg/kg to 25 mg/kg body weight. Depending upon the half-life of the anti-fibrotic agent in the particular subject, the anti-fibrotic agent can be administered between several times per day to once a week. The methods of the present invention provide for single as well as multiple administrations, given either simultaneously or over an extended period of time.

### Therapeutic Formulations

The lipocalin 2 compounds of the present invention can be formulated and administered in a variety of ways, e.g., those routes known for specific indications, including, but not limited to, topically, orally, subcutaneously, intravenously, intracerebrally, intranasally, transdermally, intraperitoneally, intramuscularly, intrapulmonary, vaginally, rectally, intraarterially, intralesionally, parenterally, intraventricularly in the brain, or intraocularly. The lipocalin 2 compound can be in the form of a pill, tablet, capsule, liquid, or sustained release tablet for oral administration; or a liquid for intravenous, subcutaneous or administration; or a polymer or other sustained release vehicle for local administration.

The lipocalin 2 compounds can be administered continuously by infusion, using a constant- or programmable-flow implantable pump, or by periodic injections. Sustained release systems can also be used. Administration can be continuous or periodic. Semipermeable, implantable membrane devices are also useful as a means for delivering lipocalin 2 in certain circumstances. For example, cells that secrete lipocalin 2 can be encapsulated, and such devices can be implanted into a subject, for example, into a primary tumor (e.g., a head and neck cancer or a pancreatic or esophageal cancer). In another embodiment, the lipocalin 2 compound is administered locally, e.g., by direct injections, when the disorder or location of the tumor permits, and the injections can be repeated periodically. Such local administration is particularly useful in the prevention and treatment of local metastasis.

Therapeutic formulations are prepared using standard methods known in the art by mixing the active ingredient having the desired degree of purity with optional physiologically acceptable carriers, excipients or stabilizers (Remington's Pharmaceutical Sciences (20^{th} edition), ed. A. Gennaro, 2000, Lippincott, Williams & Wilkins, Philadelphia, Pa.), in the form of lyophilized formulations or aqueous solutions. Acceptable carriers, include saline, or buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagines, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™, or PEG.

Optionally, but preferably, the formulation contains a pharmaceutically acceptable salt, preferably sodium chloride, and preferably at about physiological concentrations. Optionally, the formulations of the invention can contain a pharmaceutically acceptable preservative. In some embodiments the preservative concentration ranges from 0.1 to 2.0%, typically v/v. Suitable preservatives include those known in the pharmaceutical arts. Benzyl alcohol, phenol, m-cresol, methylparaben, and propylparaben are preferred preservatives. Optionally, the formulations of the invention can include a pharmaceutically acceptable surfactant. Preferred surfactants are non-ionic detergents. Preferred surfactants include Tween 20 and pluronic acid (F68). Suitable surfactant concentrations are 0.005 to 0.02%.

In one exemplary in vivo approach, the lipocalin 2 compound is a lipocalin 2 polypeptide. The lipocalin 2 polypeptide can be delivered systemically to the subject or directly to the tumor cells, e.g., to a tumor or a tumor bed following surgical excision of the tumor, in order to prevent or reduce metastasis or to inhibit survival of any remaining tumor or metastases cells. The dosage required depends on the choice of the route of administration; the nature of the formulation; the nature of the subject's illness; the subject's size, weight, surface area, age, and sex; other drugs being administered; and the judgment of the attending physician. Wide variations in the needed dosage are to be expected in view of the variety of polypeptides and fragments available and the differing efficiencies of various routes of administration. For example, oral administration would be expected to require higher dosages than administration by intravenous injection. Variations in these dosage levels can be adjusted using standard empirical routines for optimization as is well understood in the art. Administrations can be single or multiple (e.g., 2-, 3-, 6-, 8-, 10-, 20-, 50-, 100-, 150-, or more). Encapsulation of the polypeptide in a suitable delivery vehicle (e.g., polymeric microparticles or implantable devices) may increase the efficiency of delivery, particularly for oral delivery.

Alternatively, a polynucleotide containing a nucleic acid sequence encoding a lipocalin 2 polypeptide can be delivered to the appropriate cells in the subject. Expression of the coding sequence can be directed to any cell in the body of the subject. In certain embodiments, expression of the coding sequence can be directed to the tumor or metastases themselves. This can be achieved by, for example, the use of polymeric, biodegradable microparticle or microcapsule delivery devices known in the art.

The nucleic acid can be introduced into the cells by any means appropriate for the vector employed. Many such methods are well known in the art (Sambrook et al., supra, and Watson et al., Recombinant DNA, Chapter 12, 2d edition, Scientific American Books, 1992). Examples of methods of gene delivery include liposome mediated transfection, electroporation, calcium phosphate/DEAE dextran methods, gene gun, and microinjection.

In gene therapy applications, genes are introduced into cells in order to achieve in vivo synthesis of a therapeutically effective genetic product. “Gene therapy” includes both conventional gene therapy where a lasting effect is achieved by a single treatment, and the administration of gene therapeutic agents, which involves the one time or repeated administration of a therapeutically effective DNA or mRNA. Standard gene therapy methods typically allow for transient protein expression at the target site ranging from several hours to several weeks. Re-application of the nucleic acid can be utilized as needed to provide additional periods of expression of lipocalin 2.

Another way to achieve uptake of the nucleic acid is using liposomes, prepared by standard methods. The vectors can be incorporated alone into these delivery vehicles or co-incorporated with tissue-specific or tumor-specific antibodies. Alternatively, one can prepare a molecular conjugate composed of a plasmid or other vector attached to poly-L-lysine by electrostatic or covalent forces. Poly-L-lysine binds to a ligand that can bind to a receptor on target cells (Cristiano et al., J. Mol. Med. 73:479, 1995). Alternatively, tissue specific targeting can be achieved by the use of tissue-specific transcriptional regulatory elements which are known in the art. Delivery of “naked DNA” (i.e., without a delivery vehicle) to an intramuscular, intradermal, or subcutaneous site is another means to achieve in vivo expression.

Gene delivery using viral vectors such as adenoviral, retroviral, lentiviral, or adeno-associated viral vectors can also be used. Numerous vectors useful for this purpose are generally known and have been described (Miller, Human Gene Therapy 15:14, 1990; Friedman, Science 244:1275-1281, 1989; Eglitis and Anderson, BioTechniques 6:608-614, 1988; Tolstoshev and Anderson, Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller and Rosman, Biotechniques 7:980-990, 1989; Rosenberg et al., N. Engl. J. Med 323:370, 1990; Groves et al., Nature, 362:453-457, 1993; Horrelou et al., Neuron, 5:393-402, 1990; Jiao et al., Nature 362:450-453, 1993; Davidson et al., Nature Genetics 3:2219-2223, 1993; Rubinson et al., Nature Genetics 33, 401-406, 2003; and U.S. Pat. Nos. 6,180,613; 6,410,010; and 5,399,346 all hereby incorporated by reference). These vectors include adenoviral vectors and adeno-associated virus-derived vectors, retroviral vectors (e.g., Moloney Murine Leukemia virus based vectors, Spleen Necrosis Virus based vectors, Friend Murine Leukemia based vectors, lentivirus based vectors (Lois et al., Science, 295:868-872, 2002; Rubinson et al., supra), papova virus based vectors (e.g., SV40 viral vectors), Herpes-Virus based vectors, viral vectors that contain or display the Vesicular Stomatitis Virus G-glycoprotein Spike, Semliki-Forest virus based vectors, Hepadnavirus based vectors, and Baculovirus based vectors.

In the relevant polynucleotides (e.g., expression vectors), the nucleic acid sequence encoding the lipocalin 2 polypeptide (including an initiator methionine and optionally a targeting sequence) is operatively linked to a promoter or enhancer-promoter combination. Short amino acid sequences can act as signals to direct proteins to specific intracellular compartments. Such signal sequences are described in detail in U.S. Pat. No. 5,827,516, incorporated herein by reference in its entirety.

An ex vivo strategy can also be used for therapeutic applications. Ex vivo strategies involve transfecting or transducing cells obtained from the subject with a polynucleotide encoding a lipocalin 2 polypeptide. The transfected or transduced cells are then returned to the subject. The cells can be any of a wide range of types including, without limitation, hemopoietic cells (e.g., bone marrow cells, macrophages, monocytes, dendritic cells, T cells, or B cells), fibroblasts, epithelial cells, endothelial cells, keratinocytes, or muscle cells. Such cells act as a source of the lipocalin 2 polypeptide for as long as they survive in the subject. Alternatively, tumor cells (e.g., any of those listed herein), preferably obtained from the subject but potentially from an individual other than the subject, can be transfected or transformed by a vector encoding a lipocalin 2 polypeptide. The tumor cells, preferably treated with an agent (e.g., ionizing irradiation) that ablates their proliferative capacity, are then introduced into the patient, where they secrete exogenous lipocalin 2.

The ex vivo methods include the steps of harvesting cells from a subject, culturing the cells, transducing them with an expression vector, and maintaining the cells under conditions suitable for expression of the lipocalin 2 polypeptide or functional fragment. These methods are known in the art of molecular biology. The transduction step is accomplished by any standard means used for ex vivo gene therapy including calcium phosphate, lipofection, electroporation, viral infection, and biolistic gene transfer. Alternatively, liposomes or polymeric microparticles can be used. Cells that have been successfully transduced can then be selected, for example, for expression of the coding sequence or of a drug resistance gene. The cells may then be lethally irradiated (if desired) and injected or implanted into the patient.

The dosage and the timing of administering the compound depends on various clinical factors including the overall health of the subject and the severity of the symptoms of a metastatic disease, angiogenic disorder, or fibrotic disorder. In general, once a tumor, metastatic disease, or a propensity to develop a tumor or metastatic is detected, any of the methods for administering the compound described herein can be used to treat or prevent further progression of the condition. For example, continuous systemic infusion or periodic injection to the site of the tumor or metastasis of the lipocalin 2 polypeptide, or fragments or derivatives thereof, can be used to treat or prevent the disorder. Treatment can be continued for a period of time ranging from 1 day through the lifetime of the subject, more preferably 1 to 100 days, and most preferably 1 to 20 days. Dosages vary depending on the compound and the severity of the condition and are titrated to achieve a steady-state blood serum concentration ranging from 1 to 500 μg/mL lipocalin 2, preferably 1 to 100 μg/mL, more preferably 5 to 50 μg/mL and most preferably 10 to 25 μg/mL lipocalin 2.

Where sustained release administration of a lipocalin 2 polypeptide is desired in a formulation with release characteristics suitable for the treatment of any disease or disorder requiring administration of the lipocalin 2 polypeptide, microencapsulation of the lipocalin 2 polypeptide is contemplated. Micro encapsulation of recombinant proteins for sustained release has been successfully performed with human growth hormone (rhGH), interferon-(rhIFN-), interleukin-2, and MN rgp120. Johnson et al., Nat. Med., 2:795-799, 1996; Yasuda, Biomed. Ther., 27:1221-1223, 1993; Hora et al., Bio/Technology, 8:755-758 1990; Cleland, “Design and Production of Single Immunization Vaccines Using Polylactide Polyglycolide Microsphere Systems,” in “Vaccine Design: The Subunit and Adjuvant Approach,” Powell and Newman, eds., Plenum Press: New York, pp. 439-462, 1995; WO 97/03692; WO 96/40072; WO 96/07399; and U.S. Pat. No. 5,654,010.

The sustained-release formulations may include those developed using ply-lactic-coglycolic acid (PLGA) polymer. The degradation products of PLGA, lactic and glycolic acids, can be cleared quickly within the human body. Moreover, the degradability of this polymer can be adjusted from months to years depending on its molecular weight and composition. See Lewis, “Controlled release of bioactive agents from lactide/glycolide polymer,” in M. Chasin and Dr. Langer (Eds.), Biodegradable Polymers as Drug Delivery Systems (Marcel Dekker: New York, pp. 1-41, 1990.

The lipocalin 2 for use in the present invention may also be modified in a way to form a chimeric molecule comprising lipocalin 2 fused to another, heterologous polypeptide or amino acid sequence, such as an Fc sequence or an additional therapeutic molecule (e.g., a chemotherapeutic or cytotoxic agent).

The lipocalin 2 compound can be packaged alone or in combination with other therapeutic compounds as a kit. Non-limiting examples include kits that contain, e.g., two pills, a pill, and a powder, a suppository and a liquid in a vial, two topical creams, etc.

The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

Additional information on lipocalin 2 therapeutic formulations and dosages can be found in U.S. Patent Application Publication No. 20050261191.

### Diagnostics

The present invention features methods and compositions for the diagnosis of a metastatic disease, an angiogenic disease, a fibrotic disorder, or the propensity to develop such a condition using lipocalin 2 nucleic acid molecules and polypeptides. The methods and compositions can include the measurement of lipocalin 2 polypeptides, either free or bound to another molecule, or any fragments or derivatives thereof. Alterations in lipocalin 2 expression or biological activity in a test sample as compared to a normal reference can be used to diagnose any of the disorders of the invention. For example, relatively low lipocalin 2 levels may be diagnostic for solid tumors more prone to metastasize as shown by Lee et al., supra, for colon cancer cell lines.

A subject having a metastatic disease, or a propensity to develop such a condition will show an alteration (e.g., 10%, 20%, 30%, 40%, 50%, 60%, 70%, 80%, 90%, or more), preferably a decrease, in the expression of a lipocalin 2 polypeptide. The lipocalin 2 polypeptide can include full-length lipocalin 2 polypeptide, degradation products, alternatively spliced isoforms of lipocalin 2 polypeptide, enzymatic cleavage products of lipocalin 2 polypeptide, and the like. An antibody that specifically binds a lipocalin 2 polypeptide may be used for the diagnosis of a metastatic disease or to identify a subject at risk of developing such conditions.

Diagnostic methods can include measurement of absolute levels of lipocalin 2 or relative levels of lipocalin 2 as compared to a reference sample. Normal levels of lipocalin 2 found in the urine and blood samples are described in Mishra et al., Lancet 365:1205-1206 (2005) and generally range between 1-3 ng/ml. Exemplary diagnostic methods are described in U.S. Patent Application Publication No. 20050272101.

Standard methods may be used to measure levels of lipocalin 2 polypeptide in any bodily fluid, including, but not limited to, urine, blood, serum, plasma, saliva, amniotic fluid, or cerebrospinal fluid. Such methods include immunoassay, ELISA, western blotting using antibodies directed to lipocalin 2 polypeptide, and quantitative enzyme immunoassay techniques. ELISA assays are the preferred method for measuring levels of lipocalin 2 polypeptide. Alterations in the levels of lipocalin 2 polypeptide, as compared to normal controls, are considered a positive indicator of a metastatic disease, or the propensity to develop such a condition. Additionally, any detectable alteration in levels of lipocalin 2 polypeptide relative to normal levels is indicative of a metastatic disease, or the propensity to develop such a condition.

Lipocalin 2 nucleic acid molecules, or substantially identical fragments thereof, or fragments or oligonucleotides of lipocalin 2 that hybridize to lipocalin 2 at high stringency may be used as a probe to monitor expression of lipocalin 2 nucleic acid molecules in the diagnostic methods of the invention. Any of the lipocalin 2 nucleic acid molecules above can also be used to identify subjects having a genetic variation, mutation, or polymorphism in a lipocalin 2 nucleic acid molecule that are indicative of a predisposition to develop the conditions. These polymorphisms may affect lipocalin 2 nucleic acid or polypeptide expression levels or biological activity. Detection of genetic variation, mutation, or polymorphism relative to a normal, reference sample can be used as a diagnostic indicator of a metastatic disease, or the propensity to develop such a condition.

Such genetic alterations may be present in the promoter sequence, an open reading frame, intronic sequence, or untranslated 3′ region of a lipocalin 2 gene. Information related to genetic alterations can be used to diagnose a subject as having a metastatic disease, or a propensity to develop such a condition. As noted throughout, specific alterations in the levels of biological activity of lipocalin 2 can be correlated with the likelihood of a metastatic disease, or the predisposition to the same. As a result, one skilled in the art, having detected a given mutation, can then assay one or more metrics of the biological activity of the protein to determine if the mutation causes or increases the likelihood of a metastatic disease.

In one embodiment, a subject having a metastatic disease, or a propensity to develop such a condition will show a decrease in the expression of a nucleic acid encoding lipocalin 2 or an alteration in lipocalin 2 polypeptide levels. Methods for detecting such alterations are standard in the art and are described in Ausubel et al., supra. In one example Northern blotting or real-time PCR is used to detect lipocalin 2 mRNA levels.

In another embodiment, hybridization at high stringency with PCR probes that are capable of detecting a lipocalin 2 nucleic acid molecule, including genomic sequences, or closely related molecules, may be used to hybridize to a nucleic acid sequence derived from a subject having a metastatic disease or at risk of developing a such condition. The specificity of the probe, whether it is made from a highly specific region, e.g., the 5′ regulatory region, or from a less specific region, e.g., a conserved motif, and the stringency of the hybridization or amplification (maximal, high, intermediate, or low), determine whether the probe hybridizes to a naturally occurring sequence, allelic variants, or other related sequences. Hybridization techniques may be used to identify mutations indicative of a metastatic disease in a lipocalin 2 nucleic acid molecule, or may be used to monitor expression levels of a gene encoding a lipocalin 2 polypeptide (for example, by Northern analysis, Ausubel et al., supra).

In one embodiment, the level of lipocalin 2 polypeptide or nucleic acid, or any combination thereof, is measured at least two different times and an alteration in the levels (e.g., by 10%, 20%, 30%, 40%, 50%, 60%, 70%, 80%, 90%, or more) over time is used as an indicator of a metastatic disease, or the propensity to develop such a condition.

The level of lipocalin 2 polypeptide in the bodily fluids of a subject having a metastatic disease, or the propensity to develop such a condition may be altered, e.g., decreased, by as little as 10%, 20%, 30%, or 40%, or by as much as 50%, 60%, 70%, 80%, or 90% or more, relative to the level of lipocalin 2 polypeptide in a normal control reference.

In one embodiment, a subject sample of a tissue, bodily fluid, or a cell is collected soon after the diagnosis of cancer in the subject but prior to the onset of a metastatic disease. Non-limiting examples include epithelial cells from the solid tumor.

The diagnostic methods described herein can be used individually or in combination with any other diagnostic method described herein for a more accurate diagnosis of the presence of, severity of, or estimated time of a metastatic disease. In additional preferred embodiments, other known diagnostic methods for metastatic diseases can be used in combination with the methods described herein.

### Diagnostic Kits

The invention also provides for a diagnostic test kit. For example, a diagnostic test kit can include antibodies that specifically bind to lipocalin 2 polypeptide, and means for detecting, and more preferably evaluating binding between the antibodies and the lipocalin 2 polypeptide. For detection, either the antibody or the lipocalin 2 polypeptide is labeled, and either the antibody or the lipocalin 2 polypeptide is substrate-bound, such that the lipocalin 2 polypeptide-antibody interaction can be established by determining the amount of label attached to the substrate following binding between the antibody and the lipocalin 2 polypeptide. A conventional ELISA is a common, art-known method for detecting antibody-substrate interaction and can be provided with the kit of the invention. Lipocalin 2 polypeptides can be detected in virtually any bodily fluid, such as urine, plasma, blood serum, semen, or cerebrospinal fluid. A kit that determines an alteration in the level of lipocalin 2 polypeptide relative to a reference, such as the level present in a normal control, is useful as a diagnostic kit in the methods of the invention. Desirably, the kit will contain instructions for the use of the kit. In one example, the kit contains instructions for the use of the kit for the diagnosis of a metastatic disease, or the propensity to develop a metastatic disease. In another example, the kit contains instructions for the diagnosis of fibrosis, the propensity to develop fibrotic disease, angiogensis or the propensity to develop an angiogenic disorder. In yet another example, the kit contains instructions for the use of the kit to monitor therapeutic treatment or dosage regimens.

### Subject Monitoring

The diagnostic methods described herein can also be used to monitor a metastatic disease during therapy or to determine the dosages of therapeutic compounds. The diagnostic methods described herein can also be used to monitor and manage metastatic disease, angiogenic disorder, or fibrotic disorder in a subject. In this embodiment, the levels of lipocalin 2 polypeptide are measured repeatedly as a method of not only diagnosing disease but also monitoring the treatment, prevention, or management of the disease. In order to monitor the progression of a metastatic disease in a subject, subject samples are compared to control reference samples taken early in the diagnosis of cancer or a metastatic disease. Such monitoring may be useful, for example, in assessing the efficacy of a particular drug in a subject, determining dosages, or in assessing disease progression or status. For example, lipocalin 2 levels can be monitored in a patient and as levels increase, drug dosages may be decreased as well. Fernandez et al., (Clin. Cancer Res. 11:5390-5395, 2005) describe the diagnostic correlation between increased levels of lipocalin 2 in the urine samples from breast cancer patients as compared to age and sex-matched controls and the prediction of the disease status of breast cancer patients.

### Screening Assays

As discussed above, we have discovered that lipocalin 2 reverses the EMT transition and can be used to treat or prevent metastasis, angiogenic disorders, or fibrotic disorders. Based on these discoveries, compositions of the invention are useful for the high-throughput low-cost screening of candidate compounds to identify those that modulate, preferably increase (e.g., by at least 10%, 20%, 30%, 40%, 50%, 60%, 70%, 80%, 90%, 95% or more), the expression or biological activity of a lipocalin 2 polypeptide or nucleic acid molecule for the treatment of solid tumors, metastatic diseases, angiogenic disorders, or fibrotic disorders.

Any number of methods are available for carrying out screening assays to identify new candidate compounds that modulate, preferably increase, the expression of a lipocalin 2 nucleic acid molecule. In one working example, candidate compounds are added at varying concentrations to the culture medium of cultured cells expressing a lipocalin 2 nucleic acid sequence. Gene expression is then measured, for example, by microarray analysis, Northern blot analysis (Ausubel et al., Current Protocols in Molecular Biology, Wiley Interscience, New York, 2001), or RT-PCR, using any appropriate fragment prepared from the nucleic acid molecule as a hybridization probe. The level of gene expression in the presence of the candidate compound is compared to the level measured in a control culture medium lacking the candidate compound. A compound that promotes an alteration such as an increase in the expression of a lipocalin 2 gene, nucleic acid molecule, or polypeptide, or a functional equivalent thereof, is considered useful in the invention; such a molecule may be used, for example, as a therapeutic to treat a solid tumor, a metastatic disease or fibrosis, or the symptoms of a metastatic disease or fibrosis, in a subject.

In another working example, a lipocalin 2 nucleic acid is expressed as a transcriptional or translational fusion with a detectable reporter, and expressed in an isolated cell (e.g., mammalian or insect cell) under the control of a heterologous promoter, such as an inducible promoter. The cell expressing the fusion protein is then contacted with a candidate compound, and the expression of the detectable reporter in that cell is compared to the expression of the detectable reporter in an untreated control cell. A candidate compound that increases the expression of a lipocalin 2 detectable reporter is a compound that is useful for the treatment of a tumor, a metastatic disease, or fibrosis. In preferred embodiments, the candidate compound alters the expression of a reporter gene fused to a nucleic acid or nucleic acid.

In another working example, the effect of candidate compounds may be measured at the level of polypeptide expression using the same general approach and standard immunological techniques, such as western blotting or immunoprecipitation with an antibody specific for a lipocalin 2 polypeptide. For example, immunoassays may be used to detect or monitor the expression of at least one of the polypeptides of the invention in an organism. Polyclonal or monoclonal antibodies that are capable of binding to such a polypeptide may be used in any standard immunoassay format (e.g., ELISA, western blot, or RIA assay) to measure the level of the polypeptide. In some embodiments, a compound that promotes an alteration, such as an increase, in the expression or biological activity of a lipocalin 2 polypeptide is considered particularly useful. Again, such a molecule may be used, for example, as a therapeutic to delay, ameliorate, or treat a tumor, a metastatic disease, angiogenic disorder, or fibrotic disorder, or the symptoms of a tumor, a metastatic disease, angiogenic disorder, or fibrotic disorder in a subject.

In yet another working example, candidate compounds may be screened for those that specifically bind to a lipocalin 2 polypeptide or a lipocalin 2 receptor. The efficacy of such a candidate compound is dependent upon its ability to interact with such a polypeptide or a functional equivalent thereof. Such an interaction can be readily assayed using any number of standard binding techniques and functional assays (e.g., those described in Ausubel et al., supra). In one embodiment, a candidate compound may be tested in vitro for its ability to specifically bind to a lipocalin 2 polypeptide or lipocalin 2 receptor. In another embodiment, a candidate compound is tested for its ability to increase the biological activity of a lipocalin 2 polypeptide by increasing binding of a lipocalin 2 polypeptide and a siderophore or iron-siderophore.

In one particular working example, a candidate compound that binds to a lipocalin 2 polypeptide may be identified using a chromatography-based technique. For example, a recombinant lipocalin 2 may be purified by standard techniques from cells engineered to express lipocalin 2 (e.g., those described above) and may be immobilized on a column. A solution of candidate compounds is then passed through the column, and a compound specific for the lipocalin 2 polypeptide is identified on the basis of its ability to bind to the polypeptide and be immobilized on the column. To isolate the compound, the column is washed to remove non-specifically bound molecules, and the compound of interest is then released from the column and collected. Similar methods may be used to isolate a compound bound to a polypeptide microarray. Compounds isolated by this method (or any other appropriate method) may, if desired, be further purified (e.g., by high performance liquid chromatography). In addition, these candidate compounds may be tested for their ability to increase the biological activity of a lipocalin 2 polypeptide or to decrease the activity of Ras-MAPK signaling pathway (e.g., as described herein). Compounds isolated by this approach may also be used, for example, as therapeutics to treat a tumor, a metastatic disease, or fibrosis in a human subject. Compounds that are identified as binding to a polypeptide of the invention with an affinity constant less than or equal to 10 mM are considered particularly useful in the invention. Alternatively, any in vivo protein interaction detection system, for example, any two-hybrid assay may be utilized to identify compounds or proteins that bind to a polypeptide of the invention.

### Identification of New Compounds or Extracts

In general, compounds capable of increasing the activity of lipocalin 2 are identified from large libraries of both natural product or synthetic (or semi-synthetic) extracts or chemical libraries or from polypeptide or nucleic acid libraries, according to methods known in the art. Those skilled in the field of drug discovery and development will understand that the precise source of test extracts or compounds is not critical to the screening procedure(s) of the invention. Compounds used in screens may include known compounds (for example, known therapeutics used for other diseases or disorders). Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their molt-disrupting activity should be employed whenever possible.

When a crude extract is found to increase the biological activity of a lipocalin 2 polypeptide, or to bind to lipocalin 2 polypeptide, further fractionation of the positive lead extract is necessary to isolate chemical constituents responsible for the observed effect. Thus, the goal of the extraction, fractionation, and purification process is the careful characterization and identification of a chemical entity within the crude extract that increases the biological activity of a lipocalin 2 polypeptide. Methods of fractionation and purification of such heterogeneous extracts are known in the art. If desired, compounds shown to be useful as therapeutics for the treatment of a tumor, a metastatic disease, angiogenic disorder, or fibrotic disorder are chemically modified according to methods known in the art.

The following examples are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention.

### EXAMPLES

Lipocalin 2 is a member of a superfamily of carrier proteins that is expressed in granulocytic precursors as well as in numerous epithelial cells types. Lipocalin 2 binds to iron-siderophore complexes and converts embryonic kidney mesenchyme to epithelia. Downregulation of epithelial proteins and the induction of mesenchymal proteins (EMT) enhances the metastatic potential of epithelial tumors (Chambers et al., Nat. Rev. Cancer. 2:563-572, 2002; Birchmeier et al., Biochim. Biophys. Acta. 1198:11-26, 1994; Hay, Acta. Anat. (Basel) 154:8-20, 1995; Grunert et al., Nat. Rev. Mol. Cell. Biol. 4:657-665, 2003; Thiery, Nat. Rev. Cancer. 2:442-454, 2002; Fidler, Nat. Rev. Cancer. 3:453-458, 2003; Boussadia et al., Mech. Dev. 115:53-62, 2002; Islam et al., J. Cell. Biochem. 78:141-150, 2000; Thiery et al., Cancer Metastasis Rev. 18:31-42, 1999), while reactivation of epithelial genes reverses the malignant phenotype (MET) (Vanderburg et al., Acta. Anat. (Basel) 157:87-104, 1996). We hypothesized that an endogenous epithelial inducer (Yang et al., Mol. Cell. 10: 1045-1056, 2002; and Paller et al., Kidney Int. 34:474-480, 1988), lipocalin 2 could stimulate the epithelial phenotype in Ras transformed cells and reverse their metastatic potential. Though lipocalin 2 is highly expressed upon polyoma, SV 40 or neu transformation, and after malignant transformation of the breast, lung, colon, and pancreatic epithelia (Cowland et al., Genomics 45:17-23, 1997; and Friedl et al., Histochem. J. 31:433-441, 1999) its functional role in this context has been unknown. Here we demonstrate that the protein regulates the epithelial characteristics of malignant cells, as it does for embryonic mesenchyme. This activity may result from iron transport or signaling through receptors (Devireddy et al., Science 293:829-834, 2001).

To test these hypotheses, we added purified lipocalin 2 or lipocalin 2 vectors to ras transformed 4T1 mouse mammary tumor cells. These cells are known to metastasize to bone, liver, and lung tissue in a pattern similar to that found in human breast cancer (Lin et al., Proc. Natl. Acad. Sci. U.S.A. 95:8829-8834, 1998). Surprisingly, introduction of lipocalin 2 reversed ras induced EMT, reduced tumor growth, and dramatically suppressed metastasis. In lipocalin 2 treated cells, E-cadherin was rescued from proteasomal degradation by inhibition of ras-MAPK signaling. This protection was iron dependent.

The results of the experiments described in Examples 1-4, below, demonstrate that lipocalin 2 converts 4T1-ras transformed mesenchymal tumor cells to an epithelial phenotype and that lipocalin 2 can increase E-cadherin and suppress cell invasiveness in vitro and tumor growth and lung metastases in vivo and that these activities are enhanced by an iron-siderophore. Our results also demonstrate that lipocalin 2 may be reversing EMT at a point upstream of raf activation in the ras-MAPK pathway. The results of the experiments described in Examples 5-9 demonstrate that lipocalin 2 can suppress ras induced expression of VEGF in 4T1 cells via downregulation of ras-MAPK and ras-PI3K signaling and that caveolin 2 is a critical mediator of this activity. Taken together, these results demonstrate that lipocalin 2 is an inhibitor of cancer metastasis and angiogenesis. In addition, the importance of EMT in fibrosis indicates that lipocalin 2 can also be used as an inhibitor of fibrosis.

### Experimental Procedures

The following experimental procedures were used for the assays described below.

### Plasmids, Viral Constructs, Lipocalin 2 Proteins, Antibodies, and Signaling Inhibitors.

The human lipocalin 2 cDNA (GenBank accession #BC033089) with a C-terminus HA tag was PCR amplified and subcloned into pcDNA3.1 (Invitrogen, Carlsbad, Calif.). The constitutively active form H-ras A12-pBabe retroviral vector and empty-pBabe were used. Another constitutively active form of ras plasmid (H-ras V12-pcDNA3.1) was purchased from the Guthrie cDNA Resource Center (Sayre, Pa.). Constitutively active from of MEK (MEK-DD) and Lac-Z adenoviral vectors were also used and MEK-DD cDNA were also used. Caveolin-1 and antisense caveolin-1 adenovectors were gifts from Dr. Timothy C. Thompson (Baylor College of Medicine, Houston, Tex.). AKT adenovectors were gifts from Dr. Kenneth Walsh (Boston University, Boston, Mass.) (Suhara et al., Circ. Res. 89:13-19, 2001).

Recombinant mouse lipocalin 2 (accession #NM 008491) was expressed as GST-fusion protein in BL21 strain of E. coli (Stratagene, La Jolla, Calif.), which does not synthesize siderophore (Goetz et al., Mol. Cell. 10:1033-1043, 2002; and Yang et al., Mol. Cell. 10:1045-1056, 2002). Ferric sulfate (Sigma-Aldrich, St. Louis, Mo.) was added in the culture medium at 50 μM. The protein was isolated using Glutathione Sepharose 4B beads (Amersham Bioscience, Piscataway, N.J.), eluted with thrombin (Sigma-Aldrich, St. Louis, Mo.), and further purified with gel filtration (Superdex 75, Amersham Biosciences, Piscataway, N.J.). Iron-loaded (Lipo:Sid:Fe) and iron-unloaded lipocalin 2 (Lipo:Sid) were prepared by mixing the recombinant protein with iron-loaded and iron-unloaded forms of a bacterial siderophore enterochelin (EMC Microcollections, Tübingen, Germany) in PBS at room temperature for 60 minutes. Unbound siderophore was removed with Microcon YM-10 (Millipore, Bedford, Mass.). The recombinant protein diluted in culture medium was sterilized before addition to the cells using 0.22 μm filters (Millipore, Cork, Ireland).

The following reagents were purchased from respective companies: anti-ras antibody (Oncogene Research Products, San Diego, Calif.); anti-raf, anti-phospho-raf, anti-MEK1/2, anti-phospho-MEK1/2, anti-ERK1/2, and anti-phospho-ERK1/2 antibodies, anti-AKT and anti-phospho-AKT antibodies, and MEK (U0126) and PI3K inhibitors (LY294002, Cell Signaling Technologies, Beverly, Mass.); anti-E-cadherin and PY20 anti-P-Tyr monoclonal antibodies (BD Transduction Laboratories, Deerfield, Ill.); anti-vimentin monoclonal antibody and anti-caveolin-1 antibody, and FITC-conjugated goat anti-mouse IgG (Santa Cruz Biotechnology, Santa Cruz, Calif.); anti-GAPDH antibody (Chemicon International Inc, Temecula, Calif.); anti-Hakai antibody (Zymed Laboratories, San Francisco, Calif.); proteasome inhibitor MG132 (Boston Biochemistry, Cambridge, Mass.); deferoxamine mesylate salt (Sigma-Aldrich Co., St. Louis, Mo.). Anti-TSP-1 antibody was a gift from Dr. Jack Lawler (Beth Israel Deaconess Medical Center, Boston, Mass.).

### Stable Cell Lines

293T and 4T1 cells (ATCC, Manassas, Va.) were cultured in DMEM, 10% FCS and seeded (10⁶/100-mm dish) 12 hours prior to transfection with FuGene 6 reagent (32.5 μl, Roche Pharmaceuticals, Nutley, N.J.) and retroviral construct (10 μg, CA-H-ras-pBabe or empty-pBabe). 10 ml of condition were collected at 48 hours and diluted 1:1 with DMEM 10% FCS and added to the 4T1 cells (10⁶/100-mm dish) for 48 hours, followed with selective medium containing hygromycin. 8-10 single clones [4T1-ras (R) or 4T1-EV(EV)] were selected. A single clone (clone 1) from the R group was used for further studies. Similarly, a single clone (clone 1) from the EV group was selected. R cells (clone 1) were transfected with lipocalin 2-pcDNA3.1, and selected with neomycin and screened for lipocalin 2-(HA tagged) using anti-HA antibody. RL (double transfectant) clone (clone 6) which showed the highest level of lipocalin 2 expression was used for further studies.

### Measurement of VEGF Levels by ELISA

Conditioned media of 4T1 cells were collected after 2 days of incubation. Murine VEGF levels were determined in duplicate using a commercially available sandwich ELISA kit (R&D Systems, Minneapolis, Minn.), with a affinity purified polyclonal antibody specific for mouse VEGF has been pre-coated onto a microplate. Results were compared with a standard curve of mouse VEGF with a lower detection limit of 7 pg/mL. A model 680 microplate-reader (Bio-Rad Laboratories, CA) was used to measure light intensity correlating with VEGF binding.

### Immunodetection

Cells were stained as described previously (Mammoto et al., Cancer Lett. 184:165-170, 2002) and images acquired with a Delta Vision system (Applied Precision, Issaquah, Wash.) equipped with an Axiovert 100 microscope (Carl Zeiss MicroImaging Inc., Shelton, Conn.) and a Photometrics 300 series scientific-grade cooled CCD camera, reading 12-bit images, and using the 63/1.4 NA plan-Neofluar objective. For immunoprecipitation and immunoblotting, tissues were weighed, diced, soaked in ice cold RIPA buffer with 1 mM phenylmethylsulfonyl fluoride (PMSF), 1 μg/ml Aprotinin, 1 mM Na₃VO₄, 1 nM NaF, homogenized on ice, centrifuged at 10,000 g for 10 minutes at 4° C., and the supernatant fluid collected as total cell lysate. Cultured cells were washed, scraped, and solubilized in a lysis buffer containing 20 mM Tris-HCl pH 7.5, 150 mM NaCl, 0.5% Triton X-100, 1% aprotinin, and 1 mM PMSF. After 20 minutes on ice, the cells were pelleted by centrifugation and the supernatants were used as a cell lysate. Cell lysates or immunoprecipitated cell lysates were separated by PAGE (NuPAGE® gels; Invitrogen, Carlsbad, Calif.), followed by electroblotting onto a polyvinylidenedifluoride membrane (PVDF). Protein bands were detected using SuperSignal® West Pico Chemiluminescent Substrate (Pierce Chemical Co., Rockford, Ill.) (Hanai et al., J. Cell. Biol. 158:529-539, 2002).

### Luciferase Assay.

After transient transfection of the plasmids, cells were incubated for 20 hours in 10% FCS and luciferase activity in the cell lysates was determined using a luminometer normalized by sea-pansy luciferase activity under the control of the thymidine kinase promoter. The Dual-Luciferase Reporter Assay System was purchased from Promega (Madison, Wis.) (Hanai et al., J. Biol. Chem. 277:16464-16469, 2002).

### In Vitro Invasion Assay.

Polycarbonate membranes (6.5 mm diameter, 8 μm pore size) of Transwells (Coster, N.Y.) were coated with Matrigel® (BD Biosciences, Franklin Lakes, N.J.) and cells were seeded (10⁶ cells/100 μl) with DMEM including 0.1% serum. 16 hours later, cells were fixed, stained with Giemsa solution, and the upper surface of each membrane was scraped with a cotton swab. Cells that had reached the lower surface of the membrane (migrated cells) were counted in 20 random fields using a light microscope (×400).

### Semi-Quantitative Reverse Transcriptase—Polymerase Chain Reaction (RT-PCR).

Total RNA was isolated from 4T1 cells in vitro using the SV Total RNA Isolation system (Promega, Madison, Wis.). Tissue RNA was collected with TRIzol® (GibcoBRL, Gaithersburg, Md.). RT-PCR was performed on the Perkin Elmer GeneAmp PCR system 2400 using Omniscript (Qiagen, Valencia, Calif.) for reverse transcription reaction, and Taq DNA polymerase (Qiagen) and primers for mouse E-cadherin (5′-TGCCCAGAAAATGAAAAAGG-3′ and 5′-AATGGCAGGAATTTGCAATC-3′, SEQ ID NO: 5), GAPDH (5′-ACAGTCTTCTGAGTGGCA-3′, SEQ ID NO: 6, and 5′-CCCATCACCATCTTCCAG-3′, SEQ ID NO: 7) and HA-tagged lipocalin 2 (5′-GGAGTACTTCAAGATCAC-3′, SEQ ID NO: 8 and 5′-GAAAGCATAGTCTGGAACGTCATAG-3′, SEQ ID NO: 9) for DNA amplification. The PCR conditions were established for DNA amplification in the linear range. RT-PCR products were analyzed on 1% agarose gels.

For VEGF assays, two μg of each RNA sample was reverse transcribed using oligo-dT priming and control reaction was prepared for every sample where the reverse transcriptase was omitted. PCR was performed using Taq DNA polymerase (Qiagen) using primers for mouse VEGF (5′-GTA CCT CCA CCA TGC CAA GT-3′, SEQ ID NO: 10, and 5′-GCG AGT CTG TGT TTT TGC AG-3′ SEQ ID NO: 11), GAPDH (5′-ACAGTCTTCTGAGTGGCA-3′ SEQ ID NO: 6 and 5′-CCCATCACCATCTTCCAG-3′, SEQ ID NO: 7) for DNA amplification. PCR amplification was achieved by initial 94° C. incubation for 5 minutes followed by 25 cycles of 94° C. for 30 seconds, 58° C. for 30 seconds and 72° C. for 30 seconds, with 72° C. for 7 minutes as an extension time. These PCR conditions were established for DNA amplification in the linear range. PCR products were analyzed on 1.5% agarose gels.

### In Vivo Assay for Primary Tumor Growth and Pulmonary Metastases.

10⁷ 4T1-(EV, R, and RL) cells were injected subcutaneously in Balb/c mice (Asai et al., Int. J. Cancer. 76:418-422, 1998). Though this model is not the standard orthotopic model used, we have used it extensively in our laboratory to study metastases in lung. Primary tumor volume (V)=a·b·b/2, where a represents the minimum and b the maximum tumor diameter. After 3 weeks, lung weights and the number of metastatic nodules on the lung surface were evaluated.

### Statistical Analysis

All values are expressed as mean±S.E. A one tailed Student's t test was used to identify significant differences in multiple comparisons. A level of P<0.05 was considered statistically significant.

### Example 1

### Lipocalin 2 Reverses the Ras Transformed Phenotype

Numerous pathways have been defined downstream of ras activation (Campbell et al., Semin. Cancer. Biol. 14:105-114, 2004; and Downward, Nat. Rev. Cancer 3:11-22, 2003). In human tumors, ras activation typically occurs as a result of ras mutations, leaving it in a constitutively active state. The two signaling pathways studied as ras effectors include the ras-MAPK and the PI3K/Akt pathways. In the experiments described below, we demonstrate that ras-mediated EMT could be reversed by a MEK inhibitor, suggesting that the classical ras-MAPK pathway is critical for the maintenance of EMT in 4T1-ras cells. Lipocalin 2 protein reduced the phosphorylation level of raf, MEK, and ERK1/2 and the downstream activation of a reporter consisting of concatemers of the serum response element (SRE), but could not reduce SRE driven luciferase activity in the presence of a constitutively active form of MEK, suggesting that the point of lipocalin action on the ras-MAP kinase pathway was downstream of ras and upstream of MEK. Taken together with the raf phosphorylation data and the lack of change in ras expression levels, we believe that lipocalin 2 affects the ras-MAPK pathway at a point between ras and raf activation.

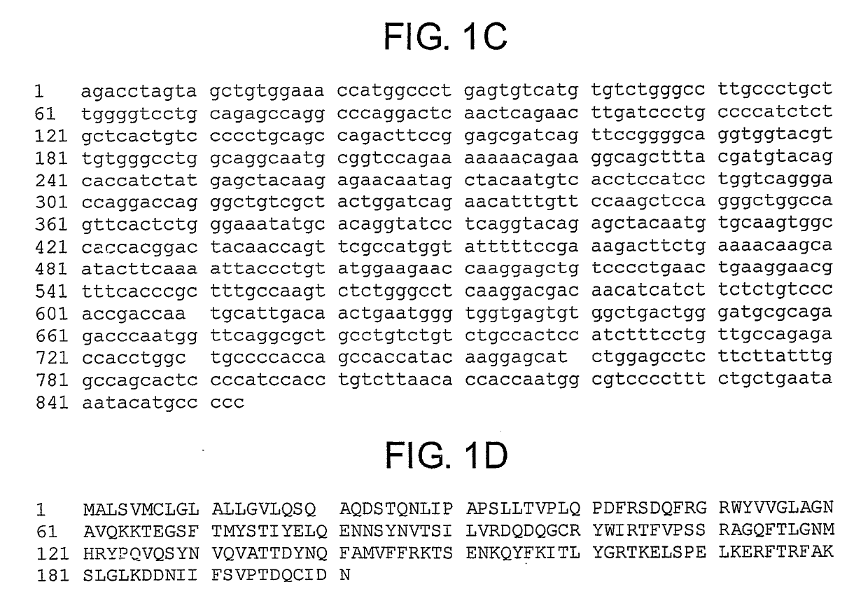

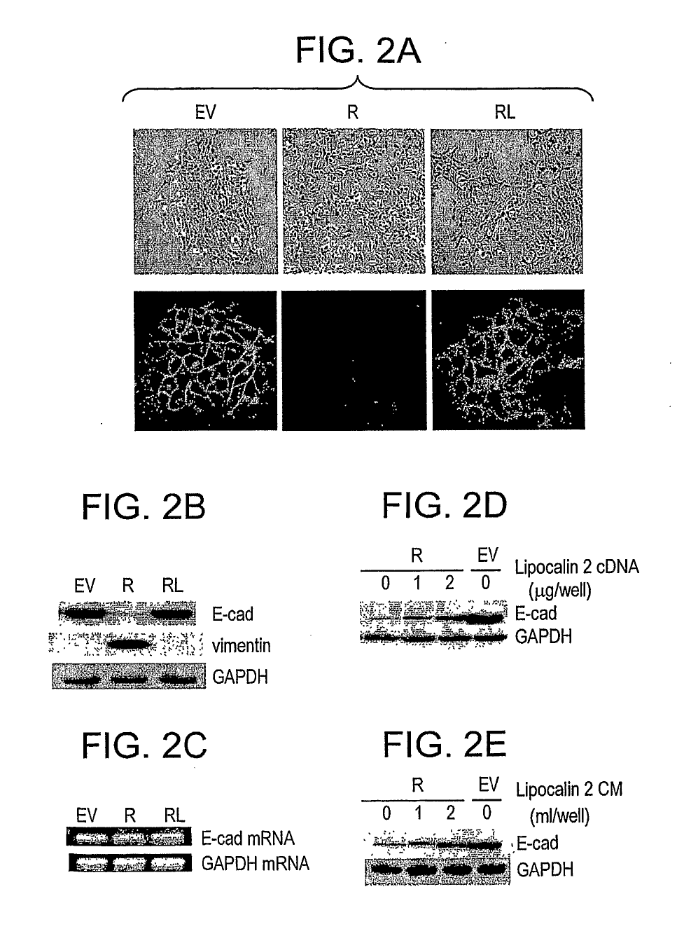

To assess the effects of lipocalin 2 on ras-mediated transformation, we chose a syngeneic spontaneously metastasizing murine breast cancer model (4T1 cell line) and accelerated its metastatic potential by introduction of constitutively active mouse H-ras mutant A12 using retrovirus. While 4T1 cells infected with an empty vector (EV) grew in a cobblestone-shaped pattern (FIG. 2A, top left), 4T1-ras (R) cells were spindle-shaped and did not form clusters at low confluency (FIG. 2A, top middle). We generated stable clones of 4T1-ras cells expressing lipocalin 2 (RL) by transfection of a lipocalin 2 expression plasmid (lipocalin 2-pcDNA3.1). Compared to R cells, the RL cells (FIG. 2A, top light) reverted to an epithelial morphology and grew appositionally (similar to EV cells), re-expressed E-cadherin and suppressed the expression of mesenchymal vimentin. (FIG. 2A, lower panel, and FIG. 2B). In contrast, E-cadherin mRNA remained unchanged (FIG. 2C), suggesting that the effect of ras and lipocalin were post-transcriptional. Expression of E-cadherin in RL cells was dependent on the dose of lipocalin 2-pcDNA3.1 expression vector (transiently introduced in a population of R cells), on a conditioned medium containing lipocalin 2 (FIGS. 2D-E), as well as on recombinant lipocalin 2 protein. R cells shown in FIG. 2D were seeded in a 6-well plate and transfected with lipocalin 2 by FuGene 6 at 40% confluency. After 48 hours, cells were trypsinized, respread on 6-well plate, and transfected again in the same conditions. After 72 hours, cells were harvested and analyzed by western blotting. EV cells in FIG. 2E were seeded in a 6-well plate with 1 ml/well containing 10% FCS with DMEM and 2 ml of CM was added at 10% confluency. After 72 hours, cells were harvested and analyzed by western blotting. CM is a mixture of media from 293T cells transfected with lipocalin 2 and from 293 cells transfected with empty vector (pcDNA3.1).

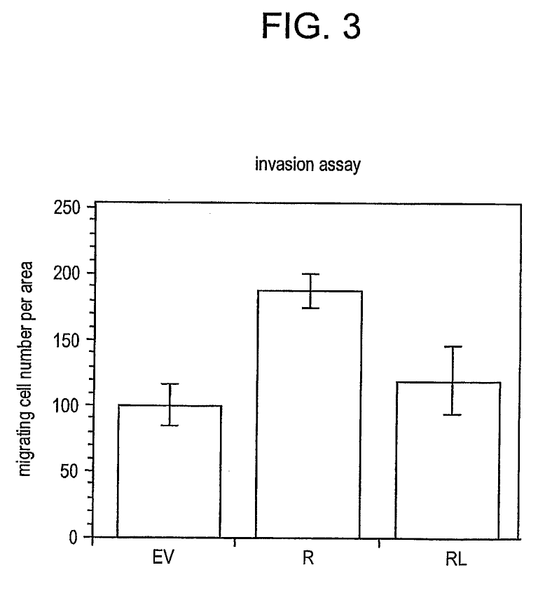

Indeed stable lipocalin 2 expression (RL) almost completely reversed (by approximately 76%) ras induced invasiveness in vitro (FIG. 3).

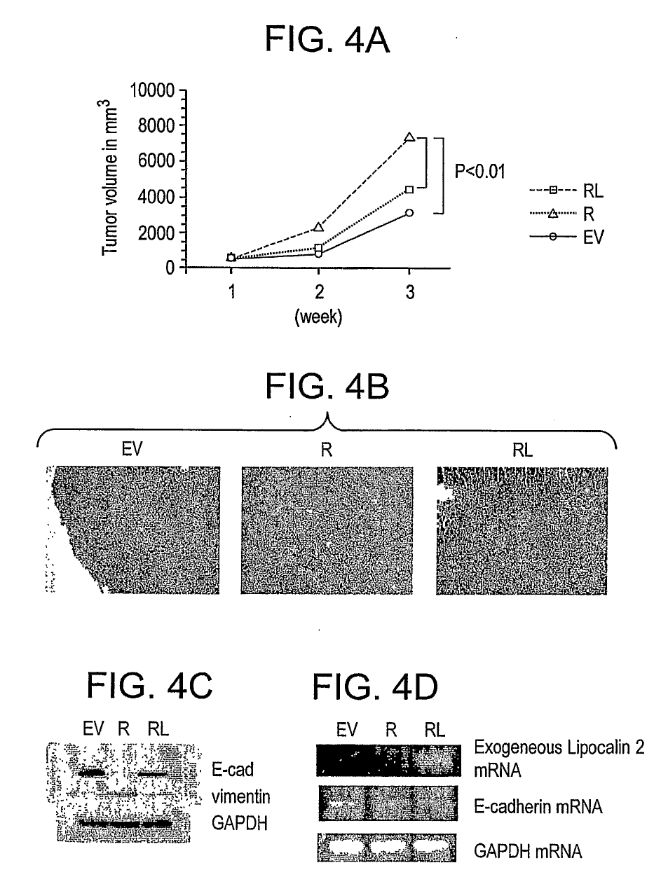

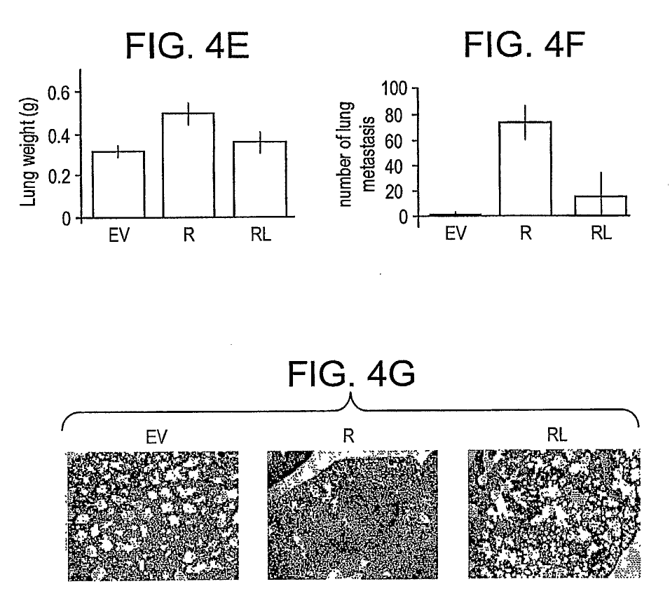

To determine whether lipocalin 2 could alter growth of tumors in vivo we injected EV, R, or RL cells subcutaneously in the backs of Balb/c mice, and assessed primary and metastatic tumor size at 1, 2, and 3 weeks post-inoculation. Primary tumors of R cells were significantly larger than lipocalin 2 cells (RL; FIG. 4A) or control cells (FIG. 4B). Lipocalin 2 reversed the soft texture, the ill-defined borders (FIG. 4B), and the invasion of adjacent muscle by the R cells. Just like control EV cells, RL tumors were solid, compact, and condensed (they could be “shelled out”). RL tumors had more E-cadherin and less vimentin than R cells, making them similar to control tumors (EV cells; FIG. 4C). Most dramatically, the number of metastatic pulmonary nodules was reduced by 80% in RL cells compared to R cells (FIGS. 4E-G) and lung weights were less. All of these effects were likely post-transcriptional; though mRNA for E-cadherin appeared downregulated in the R versus EV tumors (FIG. 4D) loading differences (note the GAPDH “controls”) make this effect less pronounced and more consistent with the in vitro data (FIG. 2C). Taken together, we find that lipocalin 2 enhanced the epithelial phenotype and inhibited metastasis of ras transformed cells.

### Example 2

### MAPK Signaling: Activation by Ras and Suppression by Lipocalin 2

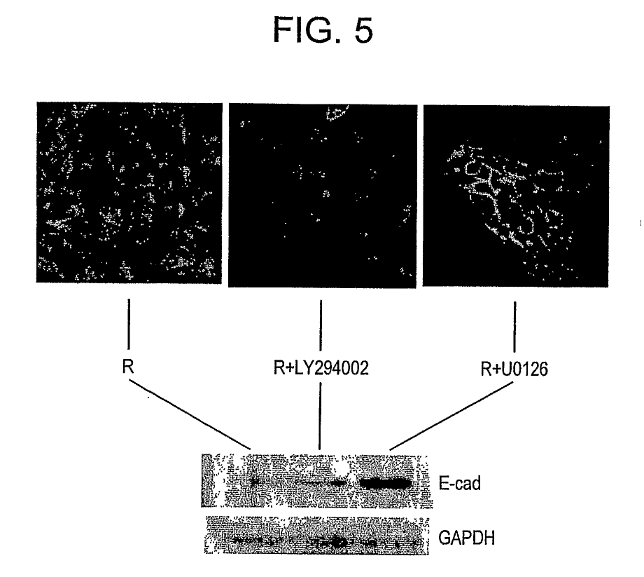

Ras has multiple downstream effectors (Campbell et al., Semin. Cancer. Biol. 14:105-114, 2004). It activates raf, which in turn activates MEK, leading to the phosphorylation of MAPK. Ras also activates PI3K. To clarify the ras pathway of EMT, we assessed the effect of a MEK inhibitor (U0126) and a PI3K inhibitor (LY294002) on R cells. As shown in FIGS. 5A-5B, the MEK inhibitor reversed ras-induced EMT, but the effect of the PI3K inhibitor was partial. Because U0126 can inhibit MEK5 in addition to the MEK1/2 (being referred to here as MEK), we infected R cells with an adenovirus carrying a dominant negative form of MEK1 and found the same results as those obtained with U0126. These data indicate that ras-MEK signaling is essential for EMT.

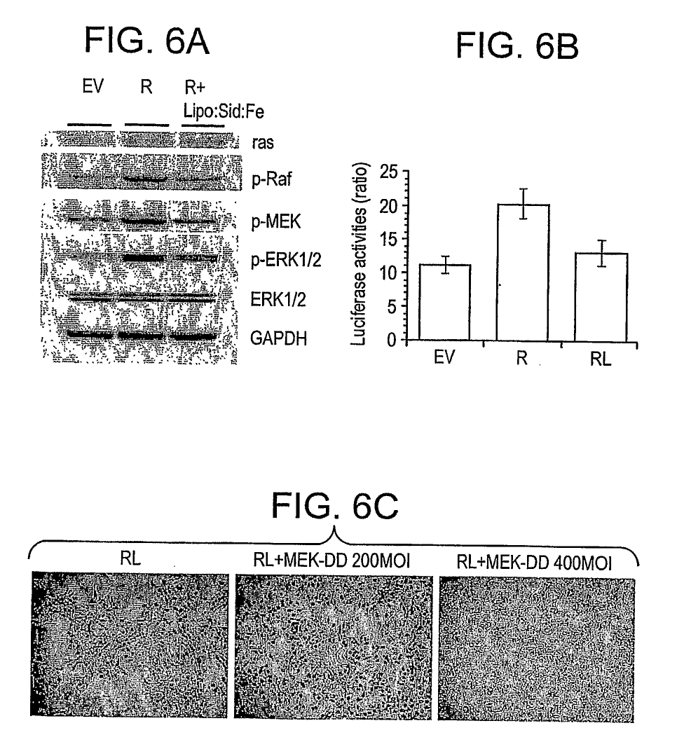

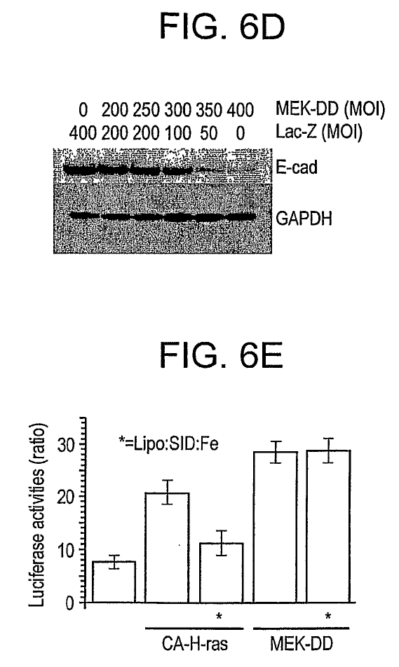

To determine whether lipocalin 2 reverted ras-induced EMT by interfering with MEK signaling, we added purified lipocalin 2 protein (iron-loaded with siderophore, Lipo:Sid:Fe) to R cells and found that ras induced phosphorylation of raf, MEK, and ERK1/2, was largely abrogated, but that total ras expression was unchanged (FIG. 6A). For these experiments, EV and R cells were starved in DMEM without serum for 48 hours. During this time, half of the R cells were incubated with 50 μg/ml of lipocalin 2 protein with iron-loaded siderophore (R+Lipo:Sid:Fe), after which all cells were incubated with 10% FCS containing DMEM for 20 minutes and then harvested for western blotting with phosphospecific antibodies. Signaling events downstream of ERK activation were then monitored with a multi-copy serum-response element (SRE)-luciferase construct introduced into EV, R, and RL (FIG. 6B). For these experiments, an SRE-luciferase assay was performed on 4T1 clones after the 48 hour incubation in serum free DMEM. The RL and EV cells gave comparable levels of luciferase activity, but this was only about half to two-thirds of the transcription found in R cells. Just like R cells treated with exogenous protein (FIG. 6A), R cells infection with recombinant adenovirus carrying lipocalin 2, but not GFP, reduced SRE-luciferase activity, MEK, and ERK1/2 phosphorylation, without altering ras expression. These data indicate that ras-MEK is modulated by lipocalin 2.

To localize the effect of lipocalin on ras-MAPK signaling, we utilized an adenovirus and expression plasmid encoding a constitutively active MEK (MEK-DD) (Murakami et al., Cell Growth Differ 10:333-342, 1999). MEK-DD adenoviral infection of EV cells led to increased SRE-luciferase activity (increased MAPK activity). Importantly, constitutively active MEK resulted in a concentration dependent EMT, as ascertained by cell shape and colony morphology (FIG. 6C) and by expression of E-cadherin protein (FIG. 6D) in RL cells, indicating that MEK-DD was dominant over the effect of lipocalin 2. RL cells in a 6-well plate were infected with an adenovirus carrying the MEK dominant active form (MEK-DD) and a Lac-Z adenovirus at the indicated multiplicities (MOI) in 2% serum including DMEM medium for 48 hours. Cells were then trypsinized, respread on 6-well plate at 5-10% confluency, and incubated with 10% serum including DMEM medium. Cell lysates were collected for western blotting 48 hours after the final plating (FIG. 6D). Consistent with this idea, MEK-DD also increased SRE-luciferase activity in EV cells, but lipocalin 2 protein (Lipo:Sid:Fe) was unable to inhibit this effect (FIG. 6E, lanes 1, and 4-5). For these assays, plasmids coding for the constitutively active form of H-ras V12 (CA-H-ras) and/or a constitutively active form of MEK pcDNA3.1 (MEK-DD) were transfected 2 hours before the protein loading. 24 hours later, cells were incubated in serum free DMEM in the presence of Lipo:Sid:Fe for another 24 hours.

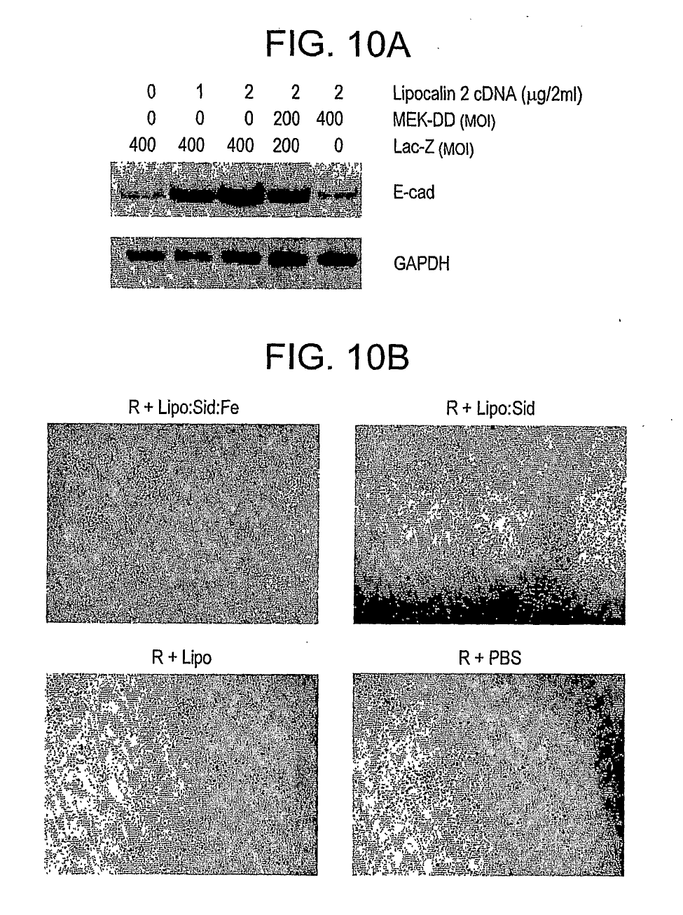

On the other hand, lipocalin 2 protein downregulated SRE-luciferase activity resulting from transfection of a constitutively active form of H-ras V12 (CA-H-ras) (FIG. 6E, lanes 1-3), as would be expected from the data with stable clones in FIG. 6B. Also, lipocalin 2 cDNA transfection induced E-cadherin expression in EV cells, but this effect was reversed by concomitant MEK-DD adenoviral infection (see FIG. 10A). These data indicate that lipocalin 2 acts upstream of MEK activation. Given that lipocalin 2 downregulated raf phosphorylation (FIG. 6A), but did not alter the level of ras expression, our data indicate that lipocalin 2 acts on ras-MAPK signaling between ras and raf. Further, events outside the ras-MAPK pathway affected by lipocalin are not sufficient to inhibit ras mediated EMT.

### Example 3

### Lipocalin 2 Inhibits Ras Induced E-Cadherin Phosphorylation and Degradation

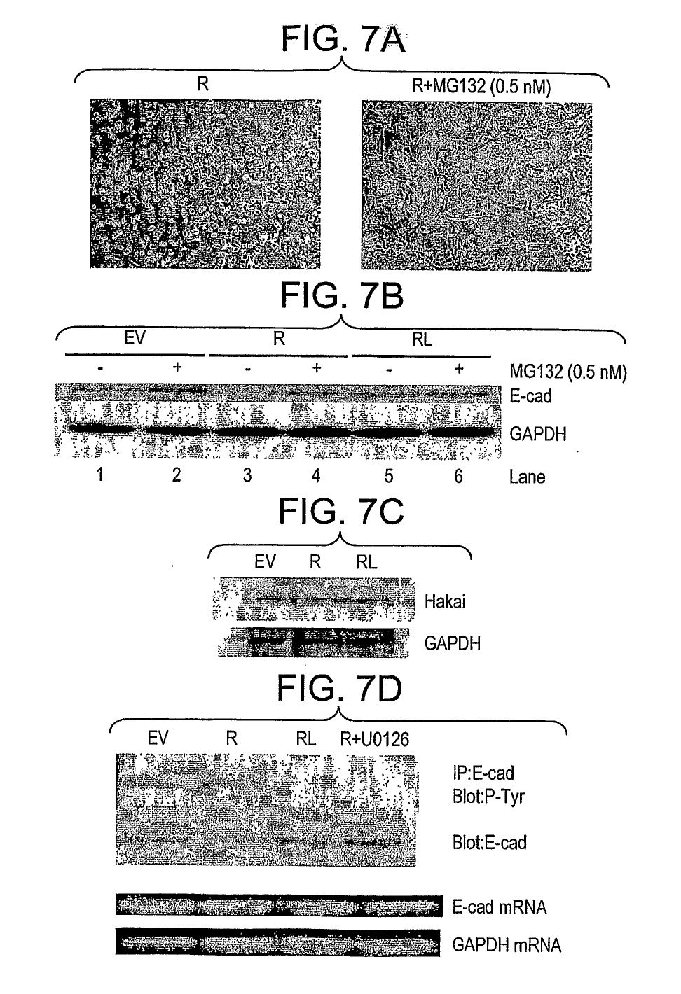

To determine how lipocalin affects ras mediated EMT, we focused on the expression of E-cadherin and its relationship to MAPK signaling. We believe lipocalin 2 modulates E-cadherin expression on a post-transcriptional level because we found it did not affect E-cadherin mRNA levels (FIGS. 2C and 4D) nor did it enhance E-cadherin promoter transcriptional activity. Indeed, we found that E-cadherin is powerfully regulated by proteosomal-mediated degradation, because proteasome inhibitor MG132 (0.5 nM) for 2 days increased E-cadherin protein in R cells (FIG. 7B, lanes 3-4) and in EV cells (FIG. 7B, lanes 1-2). In contrast, MG132 only slightly increased E-cadherin in RL cells (FIG. 7B, lanes 5-6), suggesting that E-cadherin degradation was already inhibited, and implicating lipocalin 2 in the process. There was also no significant difference in GAPDH protein expression, showing specificity and lack of toxicity of MG132. Further, it is likely that regulation of E-cadherin by proteosomal degradation is relevant to ras mediated EMT, because MG132 reverted R cells to an epithelial phenotype (FIG. 7A).

E-cadherin degradation is mediated by phosphorylation at the binding site for p120 and then recognition by Hakai (Fujita et al., Nat. Cell. Biol. 4:222-231, 2002), which targets the protein for ubiquitination and proteasomal degradation. However, Hakai expression was unchanged by ras transformation or by lipocalin 2 expression (FIG. 7C). We found that E-cadherin phosphorylation was higher in R cells than in either EV or RL cells or R cells treated with the MEK inhibitor U0126 (FIG. 7D, top panel), in a pattern inversely correlated with E-cadherin protein levels (FIG. 7D, second panel), but unaccounted for by changes in E-cadherin mRNA levels (FIG. 7D, third panel). Hence, E-cadherin phosphorylation is a target of ras signaling in 4T1 cells, that MEK activation—critical for EMT—is also responsible (directly or indirectly) for E-cadherin phosphorylation, and that lipocalin 2 impinges on the ras-MAPK pathway, suppressing E-cadherin phosphorylation, and presumably decreasing its turnover.

These experiments demonstrate that phosphorylation of E-cadherin was commensurate with a decrease in absolute levels of E-cadherin and, conversely, both lipocalin 2 as well as the MEK inhibitor markedly downregulated E-cadherin phosphorylation, while increasing the level of protein expression. Hence, MEK promotes E-cadherin phosphorylation while lipocalin 2 inhibits this pathway. Phosphorylation of E-cadherin appeared to be a critical signal for degradation, because Hakai, an ubiquitin ligase recognizes phosphorylated E-cadherin and targets it for proteasomal disposal. Consistent with this pathway, the proteasome inhibitor MG132 upregulated E-cadherin in EV cells as well as in R cells, but had minor effects on RL cells, (which might have been the result of pre-inhibition of E-cadherin degradation by lipocalin 2) and reverted the mesenchymal phenotype, suggesting that the proteasome is essential for ras-induced transformation. This is consistent with the observation that activation of the MAPK pathway promotes degradation of the γ-subunit of the epithelial Na⁺ channel (ENaC) by the proteasome pathway (Booth et al., Am. J. Physiol. Renal Physiol. 284:F938-947, 2003). Compounds that reduce E-cadherin phosphorylation or induce E-cadherin activity may also be used, alone or in combination with other compounds in the methods of the invention.

### Example 4

### Role of Iron in Lipocalin 2 Mediated Effects on E-Cadherin and MAPK Signaling

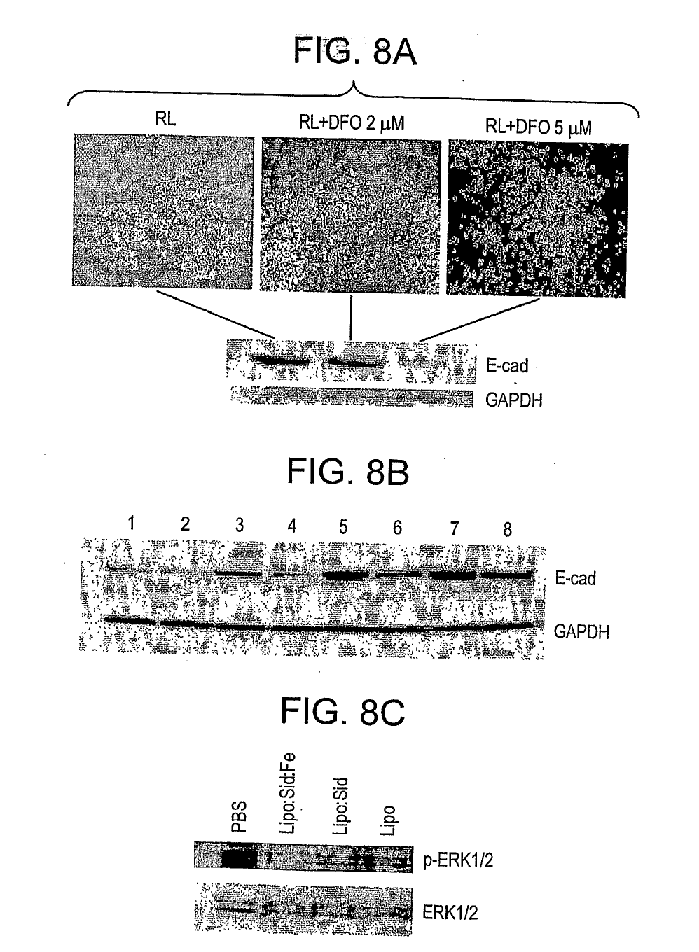

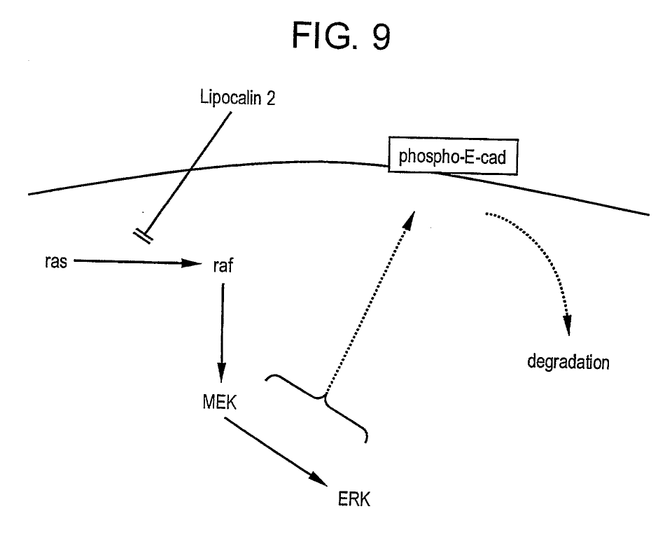

Because the inductive activity of lipocalin 2 is markedly enhanced by loading the protein with iron, we tested the effect of iron on E-cadherin expression and MAPK signaling. Deferoxamine mesylate (2-5 μM; DFO), an iron chelating agent that can deplete iron from the intracellular pool (Paller et al., Kidney Int. 34:474-480, 1988), changed the morphology of RL cells to a mesenchymal phenotype and suppressed E-cadherin expression (FIG. 8A) indicating that iron was necessary for E-cadherin expression. Indeed the effect of lipocalin 2 preparations on R cell epithelial morphology (see FIG. 10A) and E-cadherin expression correlated with iron carriage (Lipo:Sid:Fe>Lipo:Sid>Lipo; FIG. 8B and FIG. 10B) and was dose dependent. For these experiments, R cells at 40% confluency on 6-well plates were transfected with lipocalin 2-pcDNA3.1 at the indicated dose (μg/ml) using Fugene 6 and incubated for 48 hours. Cells were trypsinized and replated in 6 well plates and were transfected again under the same conditions. Cells were trypsinized and infected with MEK-DD adenovirus or a Lac-Z adenovirus in the same conditions as in FIG. 6D. Cell lysates were collected for western blotting at 48 hours after the final plating. (It should be noted that because the affinity of the siderophore for iron is so high K_{d}=10⁻⁴⁹ (Loomis et al., Inorg. Chem. 30:906-911, 1991), it is likely that the unloaded siderophore partially loaded with iron from the culture media). The same rank order was found the phosphorylation state of ERK1/2 (FIG. 7C) in cells treated with the lipocalins. In contrast to these results, simply adding iron (ferric ammonium sulfate; 50 μM) to R cells did not change their phenotype. Hence the data demonstrate that lipocalin 2 inhibits ras mediated transformation, by upregulating E-cadherin through an inhibition of MAPK signaling in an iron dependent manner, but iron alone is insufficient to reverse EMT. These data are consistent with overexpression models of E-cadherin which prevents invasiveness of human carcinoma cell lines (Grunert et al., Nat. Rev. Mol. Cell. Biol. 4:657-665, 2003; Steinberg et al., Curr. Opin. Cell. Biol. 11:554-560, 1999; Adams et al., Curr. Opin. Cell. Biol. 10:572-577, 1998; and Vanderburg et al., Acta. Anat. (Basel) 157:87-104, 1996).

The effect of lipocalin 2 on E-cadherin expression was enhanced with a siderophore and even more so with a iron-siderophore-lipocalin 2 complex. Similar data were obtained in embryonic rat mesenchyme (Yang et al., Mol. Cell. 10:1045-1056, 2002). In both of these cases, the activity of the complex might be ascribed to the siderophore, to the iron, or to the combination of any of these components with the carrier protein. First, it is most likely that the iron siderophore form is the effector, rather than the unloaded siderophore. This is because in both ras transformed cells and embryonic mesenchyme, the iron loaded form had greater activity than the iron unloaded form. Second, it is very likely that some of the iron free siderophore-lipocalin 2 complexes become partially loaded with iron in the cultures, because of their great avidity for iron (Loomis et al., Inorg. Chem. 30:906-911, 1991). These data indicate that iron enhances the actions of lipocalin 2. In fact, when we substituted iron with gallium, a metal that binds enterochelin siderophores (Loomis et al., supra), but does not undergo redox reactions that characterize iron, the induction of E-cadherin in mesenchyme was greatly diminished. Thus, compounds that enhance the stability of the siderophore-lipocalin 2 complex or which can substitute for iron to create more biologically active siderophore-lipocalin 2 complex are useful in methods of the invention. Other preferred compounds enhance lipocalin 2 intracellular release of iron. Preferred mutated or variant lipocalin 2 proteins include those with enhanced iron loading and intracellular unloading kinetics.

While not wishing to be bound by theory, it is possible that iron delivery is itself sufficient to modulate E-cadherin levels, particularly because the addition of deferoxamine mesylate (DFO) inhibited E-cadherin expression in RL cells. In agreement with this notion, DFO was found to induce phosphorylation of ERK1/2 (Kim et al., Cell Immunol. 220:96-106, 2002). However, supplying iron to R cells, in excess of the culture media, did not upregulate E-cadherin. Further, there is a report that iron overload decreases E-cadherin mRNA levels (Bilello et al., Am. J. Pathol. 162:1323-1338, 2003). It appears that different parts of the E-cadherin pathway have different sensitivities to iron loading: the ERK1/2 mediated pathway of E-cadherin degradation is iron suppressible, but de novo synthesis of E-cadherin is not iron-sensitive. Lipocalin 2 may modulate E-cadherin degradation by iron delivery, but it may be necessary to invoke a second lipocalin 2 mediated signal that initiates changes in E-cadherin levels. Indeed, lipocalin 2 suppression of ATF5 expression in lymphocytes suggests iron independent signaling by the protein.

Taken together, the results presented in Examples 1 to 4 demonstrate that lipocalin 2 can alter the invasive and metastatic behavior of ras transformed breast cancer cells—in vitro and in vivo—by reversing the EMT inducing activity of ras, through restoration of E-cadherin expression, via effects on the ras-MAPK signaling pathway. The data are consistent with overexpression models of E-cadherin which prevents invasiveness of human carcinoma cell lines (Grunert et al., Nat. Rev. Mol. Cell. Biol. 4:657-665, 2003; Steinberg et al., Curr. Opin. Cell. Biol. 11:554-560, 1999; Adams et al., Curr. Opin. Cell. Biol. 10:572-577, 1998; and Vanderburg and Hay, Acta Anat. (Basel) 157:87-104, 1996).

Prior to our discovery, the data defining the role of lipocalin 2 in the pathogenesis of cancer has been conflicting. Increased expression of lipocalin 2 was shown to accompany numerous transformations (induction by polyoma, SV40, phorbol ester and the neu oncogene), and human carcinomas (colorectal, hepatic, pancreas, breast), but the action of the protein has been obscure (reviewed in Bratt Biochim. Biophys. Acta 1482:318-326, 2000) with the exception of 2β-globulin in inducing renal cancer (Lehman-McKeeman and Caudill, Toxicol. Appl. Pharmacol. 116:170-176, 1992). One report using anti-sense RNA in an esophageal cancer cell line implanted in an animal suggested that lipocalins are tumor promoters in vivo (Li et al., Sheng Wu Hua Xue Yu Sheng Wu Wu Li Xue Bao (Shanghai) 35:247-254, 2003), and lipocalin 2 may promote slightly the proliferation of estrogen receptor negative mammary cells in vitro (Seth et al., Cancer Res. 62:4540-4544, 2002). However using a large variety of assays we find a protective role for lipocalin 2 during ras mediated transformation and metastasis in vitro and in vivo. Indeed the lipocalin 2 produced smaller, more coherent tumors of higher density (similar weight but different cell types), with less regional invasion and dramatically fewer metastases in vivo as assessed by lung weight, by the number of nodules on the lung surface, as well as by histology.

### Example 5

### Effects of Lipocalin 2 on Ras-Induced VEGF Production in 4T1 Cells In Vitro

Given our results described above demonstrating the effects on in vivo tumor growth and metastasis, we asked whether lipocalin 2 might also regulate angiogenic activity of tumor cells. To test this hypothesis, we focused primarily on VEGF expression, which is known to be induced by ras activation. Introduction of lipocalin 2 downregulated VEGF at both mRNA and protein levels via inhibition of ras-MAPK and PI3K signaling. Caveolin-1 was found to be critical in mediating both the MET and anti-angiogenic functions of lipocalin 2.

Since ras transformation is known to promote angiogenesis (Arbiser et al., Proc. Natl. Acad. Sci. U.S.A. 94:861-866, 1997), we explored whether lipocalin 2 would also reverse this action of ras. Ras is known to upregulate the production of vascular endothelial growth factor (Rak et al., Cancer Res. 60:490-498, 2000; and Kranenburg et al., Biochim. Biophys. Acta 1654:23-37, 2004), a potent pro-angiogenic protein, important in endothelial cell survival, proliferation and migration and to demonstrate the anti-angiogenic protein thrombospondin 1 (TSP-1). We show here lipocalin 2 antagonizes these pro-angiogenic activities of ras in the 4T1 cell line system, in vitro and in vivo.

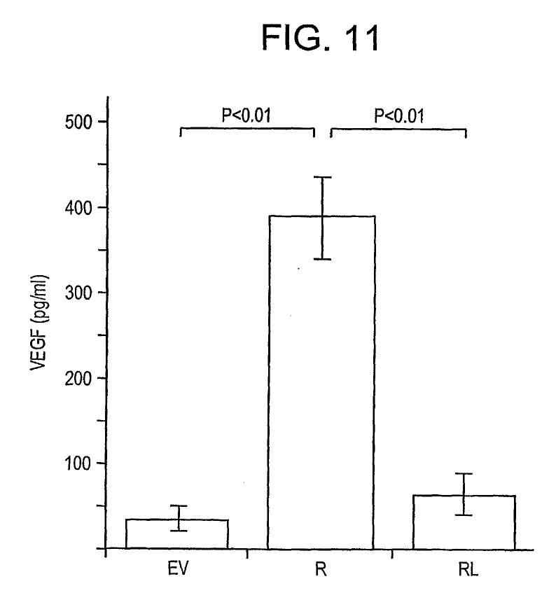

To determine the effects of lipocalin 2 on angiogenesis, we used 3 stable clones of 4T1 cells: infected with empty retroviral control (EV cells), retrovirally infected with constitutively active mouse H-ras mutant A12 (R cells), and R cells transfected with a lipocalin 2 expression plasmid (RL cells) as described above. We evaluated VEGF production from these stable cell lines by an ELISA assay. VEGF secretion from 4T1 cells (EV) was dramatically upregulated (approximately 10 fold) by ras-transformation (R) but was suppressed (≈7.5 fold) nearly to baseline in the lipocalin 2 (RL) transfectants (FIG. 11).

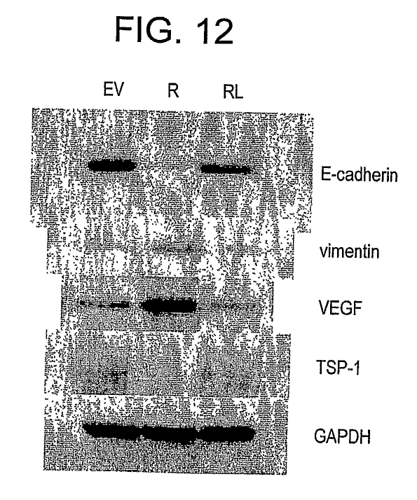

To determine whether lipocalin 2 could affect expression of anti-angiogenic factors in vivo, we injected EV, R, or RL cells subcutaneously in the backs of Balb/c mice and dissected the primary tumors at 3 weeks post-inoculation. As shown above and in Hanai et al., supra, E-cadherin and vimentin protein expression varied reciprocally in the three tumor types. We assessed the expression of VEGF and thrombospondin-1 (TSP-1) by western blot in each tumor tissue. In the primary tumors of R cells, a significantly larger amount of VEGF protein was observed as compared to tumors derived from EV cells, an increase that was completely abrogated in RL cells (FIG. 12). Moreover, the anti-angiogenic protein TSP-1 was downregulated by ras in the primary tumors of R cells, also in accordance with previous data (Watnick et al., Cancer Cell 3; 219-231, 2003) and returned in RL cells to the levels noted in EV cells. These data indicate in vivo anti-angiogenic activity of lipocalin 2 by its effects on the expression of two angiogenic molecules.

### Example 6

### Involvement of MAPK and PI3K Signaling in ras Induced VEGF Production in 4T1 Cells

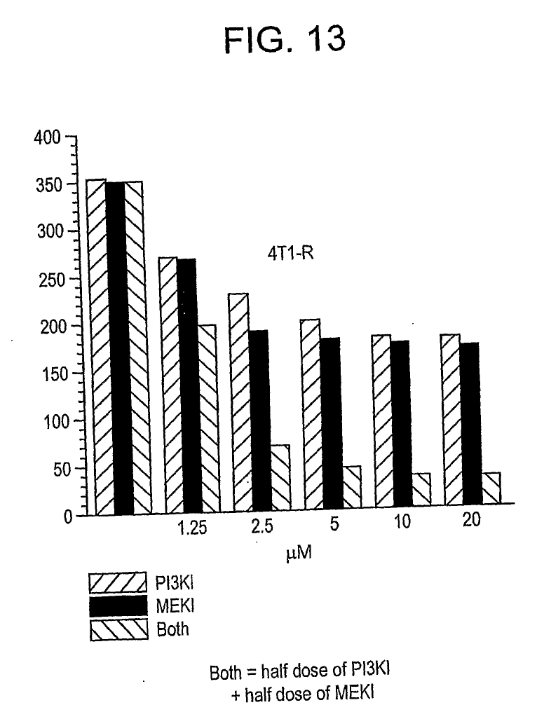

Next, we explored the mechanism by which lipocalin 2 alters VEGF expression by determining which signaling pathways known to stimulate VEGF expression in other cell types and known to be ras effectors (Pages et al., Cardiovasc. Res. 65:564-573, 2005; and Josko et al., Med. Sci. Monit. 10:RA89-98, 2004) were applicable to our 4T1 systems. We used both MEK and PI3K inhibitors to address this question. In R cells, each inhibitor alone reduced VEGF production in a dose dependent manner, with a maximum inhibition of approximately 50% (FIG. 13). However, combined blockade of these pathways showed greater than 90% inhibition (FIG. 13).

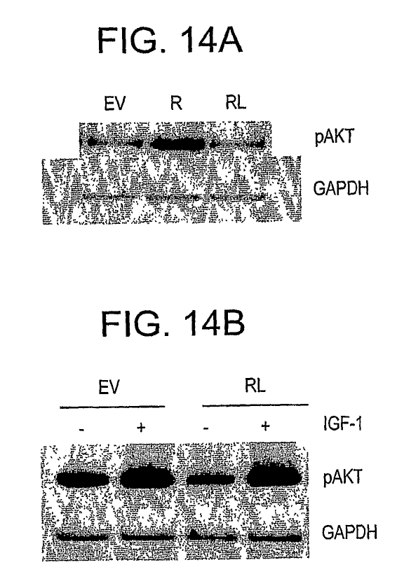

We have previously shown that lipocalin 2 downregulates MEK and ERK phosphorylations (see Examples 1-4, above and Hanai et al., supra). We therefore explored whether lipocalin 2 affects PI3K signaling. Lipocalin 2 downregulates ras-induced phosphorylation of AKT (FIG. 14A). However lipocalin 2 does not downregulate IGF-1-induced phosphorylation of AKT (FIG. 14B), suggesting that the effect of lipocalin 2 on AKT phosphorylation shows specificity to ras signaling.

### Example 7

### Lipocalin 2 Reduces the Expression of VEGF mRNA in 4T1 Cells In Vitro

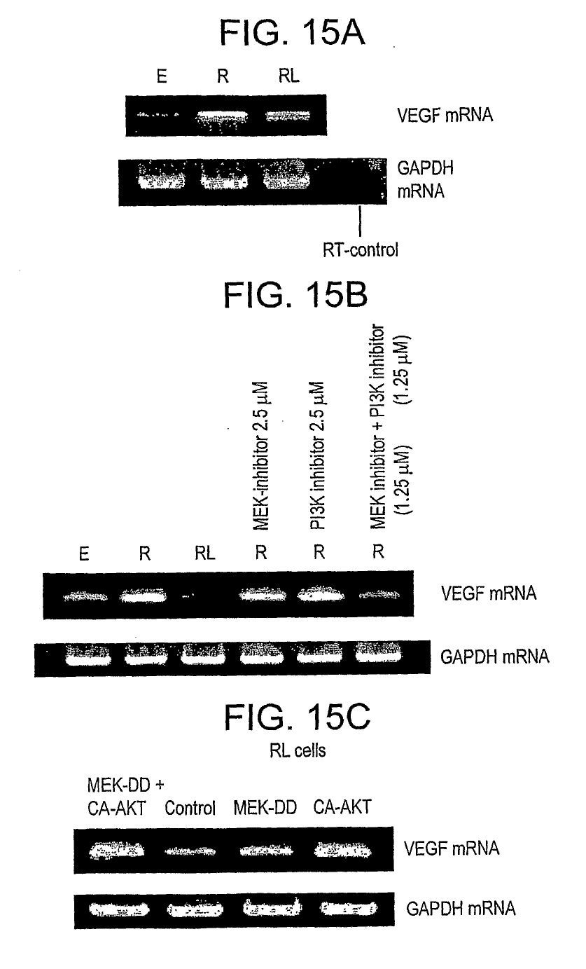

Having noted lipocalin 2's effects on VEGF protein expression and secretion, we asked whether these effects were secondary to changes in VEGF mRNA levels. We tested the effects on VEGF mRNA using 3 stable cell clones of 4T1 cells. Ras-transformation augmented VEGF mRNA level and lipocalin 2 reduced this upregulation (FIG. 15A), in agreement with our VEGF protein data (FIGS. 11 and 12).

We also observed the synergistic inhibition of VEGF mRNA expression by the PI3K inhibitor and the MEK inhibitor (FIG. 15B). These are consistent with the ELISA data shown in FIG. 13.

### Example 8

### Lipocalin 2's Inhibitory Effect on VEGF mRNA Expression is Reversed by Activation of MAPK and PI3K Signaling

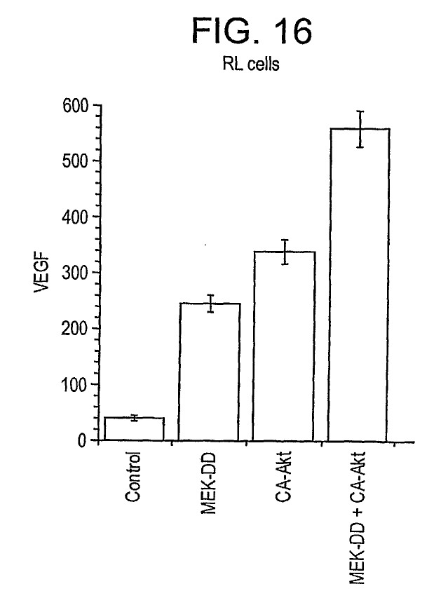

Using signaling inhibitors, we showed VEGF mRNA expression, and VEGF secretion is regulated by both PI3K and MAPK signaling and that lipocalin's effects (FIG. 15A, lane 3 or FIG. 15B lane 3) appear to mimic these seen with combined blockade of MEK and PI3K (FIG. 15B, lane 6). To determine whether lipocalin 2 functions upstream or downstream of MEK and PI3K activation by ras, and ras induced VEGF expression, we used constitutively active forms of MEK (MEK-DD) and AKT (CA-AKT) and assessed VEGF mRNA levels (FIG. 15C). CA-AKT and MEK-DD reversed the inhibitory effect of lipocalin 2 on VEGF mRNA with CA-AKT being the most potent. These effects were also qualitatively confirmed at the level of VEGF secretion by ELISA assay (FIG. 16). Though it appeared that in contrast with the VEGF mRNA data, activation of the two pathways together gave maximal VEGF secretion.

### Example 9

### Lipocalin 2 Upregulates the Expression of Caveolin-1 in 4T1 Cells

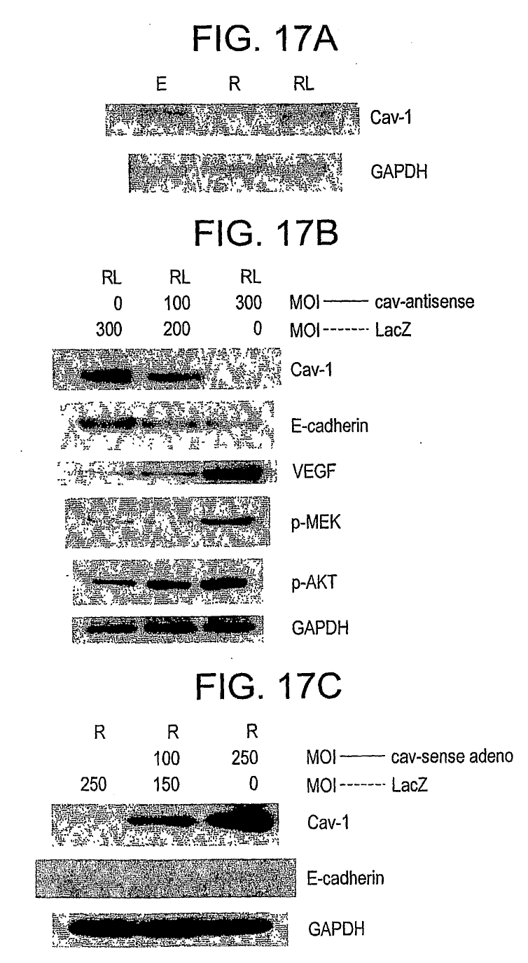

Since caveolin-1 expression is known to affect a number of signaling pathways and the loss of caveolin-1 has been associated with ras transformation (Lu et al., Cancer Cell 4:499-515, 2003), we sought to examine the effects of lipocalin 2 signaling with ras and caveolin-1 expression. Using the stable clones of 4T1 cells, we found that in the process of ras-transformation, caveolin-1 is lost, consistent with the previous findings (Engelman et al., J. Biol. Chem. 274:32333-32341, 1999; and Lu et al., supra), and lipocalin 2 rescued this loss of caveolin-1 (FIG. 17A). In the RL cells, the epithelial phenotype was lost in a dose-dependent manner by inhibition of caveolin-1 expression using adenoviral infection of a caveolin-1 antisense construct (FIG. 17B), suggesting that caveolin-1 is necessary for the EMT reversing function of lipocalin 2. Reduction in caveolin-1 expression also led to a decrease in E-cadherin expression, as noted earlier Lu et al., supra. Interestingly, VEGF expression increased dramatically as caveolin-1 expression decreased and moreover, there was a concomitant activation of pMEK and pAKT. These data implicate a role for caveolin-1 in mediating both the EMT inhibitory and anti-angiogenic activities of lipocalin 2.

We also assessed whether caveolin-1 is sufficient to induce MET. We increased the expression of caveolin-1 in R cells by adenoviral infection of a caveolin-1 construct. We found that caveolin-1 did not cause a morphologic change in the R cells, nor was E-cadherin expression increased (FIG. 17C), suggesting that caveolin-1 is not sufficient to cause MET.

The epithelial to mesenchymal transition process is known to induce autocrine signaling involving VEGF and Flt-1 and to enable invasive cells to become ‘self-sufficient’ for survival (Bates et al., Cancer Biol. Ther. 4:365-370, 2005). Our data demonstrates that VEGF was upregulated in ras-transformed 4T1 tumor cells (FIGS. 11 and 12). Thrombospondin-1, an endogenous inhibitor of angiogenesis, known to be downregulated by ras (Kranenburg et al., supra; Rak et al., supra; Viloria-Petit et al., Embo. J. 22:4091-4102, 2003; and Watnick et al., supra) was upregulated by lipocalin 2 (FIG. 12). These data suggest that lipocalin 2 has inhibitory effects on ras induced tumor angiogenesis, by restoring the balance between pro (VEGF) and anti-angiogenic (TSP-1) targets downstream of ras transformation.

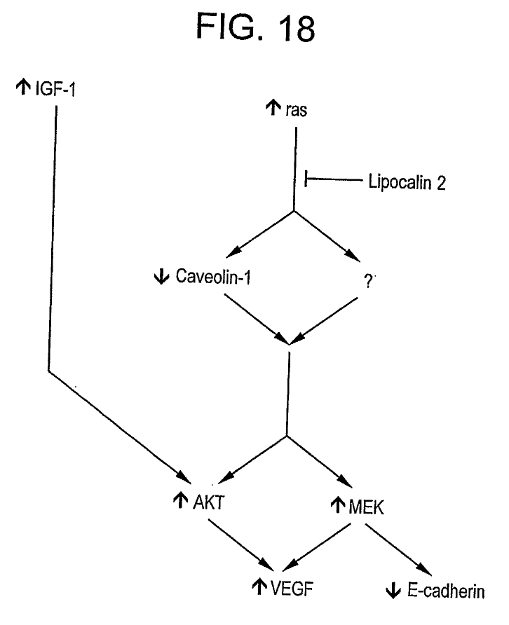

Production of VEGF by tumor is essential for the survival of tumor cells and is regulated by a variety of mechanisms (Josko et al., supra). For example, several response elements, such as HIF-1, SP-1, AP2, Egr-1 and STAT sites, have been identified for the transcriptional regulation of VEGF expression (Pages et al., supra). Our results demonstrate that lipocalin 2 reverses ras-induced transformation by targeting several of the downstream effects of ras including the upregulation of VEGF (Grugel et al., J. Biol. Chem. 270:25915-25919, 1995). VEGF secretion from 4T1 cells (EV) was dramatically upregulated by ras-transformation (R) but was suppressed nearly to baseline in the lipocalin 2 (RL) transfectants (FIG. 11). In mice, subcutaneously injected R cells, but neither EV nor RL cells, gave rise to in an area of peri-tumoral edema over a 3 day period. Our results underscore the importance of ras-MAPK, ras-PI3K, and possibly HIF-1 pathways for the regulation of VEGF expression in 4T1 cells (Josko et al., supra; and Skinner et al., J. Biol. Chem. 279:45643-45651, 2004) (see FIG. 18).

The induction of an angiogenic phenotype by oncogene activation or through the loss of tumor suppressor gene function has been well-described (Watnick et al., supra; Rak et al., supra; and Webb et al., J. Neurooncol. 50:71-87, 2000). For example, ras and src are known to induce angiogenic proteins and to repress endogenous inhibitors of angiogenesis. Ras causes potent induction of VEGF and downregulates the angiogenesis inhibitor thrombospondin-1 (Rak et al., supra; Viloria-Petit et al., supra; Watnick et al., supra; and Kranenburg et al., supra). Initial studies suggest that ras induction of VEGF may be partly mediated through the PI3 kinase pathway (Arbiser et al., Proc. Natl. Acad. Sci. U.S.A. 94:861-866, 1997; and Rak et al., supra). Similarly the loss a tumor suppressor can lead to upregulation of proangiogenic pathways. For example, loss of the VHL tumor suppressor has been known to upregulate VEGF through stabilization of HIF-1α (Turner et al., Cancer Res. 62:2957-2961, 2002). In most cases however, the detailed mechanisms by which the gain of a dominantly active oncogene or the loss of a tumor suppressor leads to a proangiogenic state has not been well-defined. We have shown in this report that ras up regulates the production of VEGF in cultured 4T1 cells. When lipocalin 2 was added, this induction was largely abrogated. Thus, lipocalin 2 reversed the proangiogenic ras induced state in 4T1 cells.

Furthermore, in an in vivo setting, the expression of an endogenous inhibitor of angiogenesis, thrombospondin-1, as well as the level of VEGF expression, and the proangiogenic state induced by ras, were all reverted by lipocalin 2 in tumor tissue.

In addition these results suggest that caveolin-1 downregulation causes upregulation of ras-MAPK signaling (FIGS. 17 and 18), consistent with previous reports (Williams et al., Am. J. Physiol. Cell. Physiol. 288:C494-506, 2005; and Cohen, Am. J. Physiol. Cell Physiol. 284:C457-474, 2004). Based on our data, we suggest that caveolin-1 is necessary for the MET induction and anti-angiogenic functions of lipocalin 2 in 4T1 tumor cells (FIG. 17B) however caveolin-1 alone may not be sufficient to cause MET (FIG. 17C).

Here again, there are contradictory reports regarding the role of caveolin-1 in tumorigenesis and metastasis (see, for example Lu et al., supra) showing that EGF downregulates caveolin-1, causing a loss of E-cadherin and tumor cell invasion. Additional papers suggest that caveolin-1 is thought to be a tumor suppressor protein (Fiucci et al., Oncogene 21:2365-2375, 2002; and Razani et al., Biochem. Soc. Trans. 29:494-499, 2001), while others suggest that up-regulated caveolin-1 is a prognostic parameter for poor survival (Ho et al., Am. J. Pathol. 161:1647-56, 2002). Based on our results, we propose that the role of caveolin-1 may be dependent on tumor developmental stages. In the early stages of tumor development caveolin-1 may act as a tumor suppressor molecule and in late and advanced stages of tumor development, it contributes to the invasive potential of the tumor cells.

Taken together, the results of the experiments described above demonstrate that lipocalin 2 can alter the angiogenic activity of 4T1 tumor cells through down regulating MAPK and PI3K pathways and that caveolin-1 is involved in the MET-inducing and anti-angiogenic activities of lipocalin 2 and the ras-MAPK pathway is involved in the anti-metastatic activities of lipocalin 2. These results further support our discovery that lipocalin 2 or lipocalin 2 compounds have a protective function in tumor angiogenesis and metastasis, and in angiogenesis, in general.

### Other Embodiments

From the foregoing description, it will be apparent that variations and modifications may be made to the invention described herein to adopt it to various usages and conditions. Such embodiments are also within the scope of the following claims.

All publications, patent applications, and patents mentioned in this specification are herein incorporated by reference to the same extent as if each independent publication, patent, or patent application was specifically and individually indicated to be incorporated by reference.

What is claimed is:

1. A method for treating or preventing metastasis in a subject having cancer, said method comprising administering to said subject a lipocalin 2 compound, or a fragment or derivative thereof, that has lipocalin 2 biological activity, wherein said administering is for a time and in an amount sufficient to treat or prevent said metastasis in said subject.

2. The method of claim 1, wherein said lipocalin 2 compound is a lipocalin 2 polypeptide or fragment thereof.

3. The method of claim 2, wherein said lipocalin 2 polypeptide comprises a sequence substantially identical to the amino acid sequence of SEQ ID NO:2 or SEQ ID NO:4.

4. The method of claim 3, wherein said lipocalin 2 polypeptide comprises the sequence of SEQ ID NO:2 or SEQ ID NO:4.

5. The method of claim 4, wherein said lipocalin 2 polypeptide consists of the amino acid sequence of SEQ ID NO:2 or SEQ ID NO: 4.

6. The method of claim 1, wherein said lipocalin 2 compound binds a siderophore.

7. The method of claim 1, wherein said lipocalin 2 compound transports iron.

8. The method of claim 1, wherein said lipocalin 2 biological activity is the reversal or inhibition of epithelial to mesechymal transition.

9. The method of claim 1, wherein said lipocalin 2 compound binds to a lipocalin 2 receptor.

10. The method of claim 1, wherein said lipocalin 2 compound decreases phosphorylation of E-cadherin, increases E-cadherin biological activity, or inhibits ras-MAPK signaling.

11. The method of claim 1, wherein lipocalin 2 compound increases E-cadherin expression.

12. The method of claim 1, further comprising administering a siderophore.

13. The method of claim 12, wherein said siderophore is selected from the group consisting of bacterial catecholate-type ferric siderophores, enterochelin, carboxymycobactin, aminochelin, desferrioxamine, aerobactin, arthrobactin, schizokinen, foroxymithine, pseudobactins, neoenactin, photobactin, ferrichrome, hemin, achromobactin, achromobactin, and rhizobactin.

14. The method of claim 12, wherein said siderophore is in a complex with said lipocalin 2 compound.

15. The method of claim 1, further comprising administering iron or an iron replacement.

16. (canceled)

17. The method of claim 15, wherein said iron or iron replacement is in a complex with said lipocalin 2 compound either directly or indirectly.

18. The method of claim 17, wherein said iron or iron replacement is in a complex with said lipocalin 2 compound prior to administering to said subject.

19. The method of claim 1, wherein said lipocalin 2 compound is a nucleic acid molecule encoding a lipocalin 2 polypeptide that has lipocalin 2 biological activity.

20-22. (canceled)

23. The method of claim 1, wherein said cancer is from an epithelial cell solid tumor.

24. The method of claim 23, wherein said epithelial cell solid tumor is a cancer selected from the group consisting of gastrointestinal cancer, colon cancer, breast cancer, prostate cancer, renal cancer, lung cancer, melanoma, ovarian cancer, pancreatic cancer, head and neck cancer, and liver cancer.

25. The method of claim 1, wherein the cancer is metastatic and said method is used to treat said metastasis.

26. The method of claim 1, wherein the cancer is at risk of becoming metastatic.

27. The method of claim 1, wherein the subject is at risk for cancer or cancer metastasis.

28. The method of claim 1, further comprising administering to said subject an additional cancer therapy selected from the group consisting of surgery, radiation therapy, chemotherapy, differentiating therapy, and immune therapy.

29-31. (canceled)

32. A kit for the treatment or prevention of metastasis in a subject having, or at risk of developing, a metastatic cancer, said kit comprising a lipocalin 2 compound and instructions for the use of said lipocalin 2 compound for the treatment or prevention of said metastatic cancer.

33. The kit of claim 32, further comprising at least one additional compound selected from the group consisting of a chemotherapeutic agent, an angiogenesis inhibitor, or an anti-proliferative compound.

34. A method for reducing or inhibiting angiogenesis in a subject in need thereof, said method comprising administering to said subject a lipocalin 2 compound, wherein said administering is for a time and in an amount sufficient to reduce or inhibit said angiogenesis.

35-51. (canceled)

52-53. (canceled)

54. A method of diagnosing metastatic disease or a propensity to develop a metastatic disease in a subject having or at risk of having cancer, said method comprising the steps of:
(a) determining the level of a lipocalin 2 polypeptide, nucleic acid molecule, or fragments thereof, in a sample from said subject; and
(b) comparing said level in (a) to a normal reference level of lipocalin 2 polypeptide, nucleic acid molecule, or fragment thereof; wherein an alteration in said subject levels relative to said normal reference level is diagnostic of a metastatic disease or a propensity to develop a metastatic disease in said subject.

55-57. (canceled)

58. The method of claim 54, wherein said method is used to monitor the metastatic health of a subject having or at risk of having cancer.

59-69. (canceled)

70. A method for treating or preventing fibrosis in a subject having a fibrotic disorder, said method comprising administering to said subject a lipocalin 2 compound, wherein said administering is for a time and in an amount sufficient to prevent or reduce the occurrence of said fibrosis.

71-77. (canceled)
